SEQUENCE LISTING (18 sequences)

SEQ ID NO: 1 - protein, 379 aa, Homo sapiens
  MISC_FEATURE (1)..(379): WT STING protein
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 2 - DNA, 1140 bp, Homo sapiens
  misc_feature (1)..(1140): WT STING coding seq
atgccccacagctctctgcaccctagcatcccttgtcctagaggacacggcgcccagaaagctgctctggttctgctgtctgcctgcctggttacactgtggggactgggagagcctcctgagcacacactgagatacctggtgctgcacctggcttctctgcagctgggactgctgctgaacggcgtgtgttctctggccgaggaactgagacacatccacagcagatacaggggcagctactggcggacagtcagagcttgtctgggctgccctcttagaagaggcgctctgctgctgctgtccatctacttctactacagcctgcctaacgctgtgggccctcctttcacatggatgctggctctgctgggcctgtctcaggctctgaatatcctgctggggctgaaaggcctggctcctgctgaaatctctgccgtgtgcgagaagggcaacttcaacgtggcacacggcctggcctggtcctactacatcggatacctgagactgatcctgcctgagctgcaggccagaatcagaacctacaaccagcactacaacaacctgctgagaggcgctgtgtcccagagactgtacatcctgctgcctctggactgtggcgtgccagacaacctgtctatggccgatcctaacatccggtttctggacaagctgcctcagcagacaggcgatcacgccggcatcaaggacagagtgtacagcaacagcatctacgagctgctggaaaacggccagcgagccggaacatgcgtgctggaatatgccacacctctgcagaccctgttcgccatgagccagtactctcaggccggcttcagcagagaggacagactggaacaggccaagctgttctgcaggaccctcgaggacatcctggctgatgctccagagagccagaacaactgcagactgatcgcctaccaagagcctgccgacgacagcagctttagcctgagccaagaggtgctgagacacctgagacaagaagagaaagaggaagtcaccgtcggcagcctgaaaaccagcgccgtgcctagcaccagcaccatgtctcaagaacccgaactgctgatctccggcatggaaaagcccctgcctctgagaaccgacttcagctag

SEQ ID NO: 3 - protein, 379 aa, Artificial Sequence
  Synthetic construct, STING N154S mutant
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFSVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 4 - DNA, 1140 bp, Artificial Sequence
  Synthetic construct, STING  N154S mutant coding seq
atgccccacagctctctgcaccctagcatcccttgtcctagaggacacggcgcccagaaagctgctctggttctgctgtctgcctgcctggttacactgtggggactgggagagcctcctgagcacacactgagatacctggtgctgcacctggcttctctgcagctgggactgctgctgaacggcgtgtgttctctggccgaggaactgagacacatccacagcagatacaggggcagctactggcggacagtcagagcttgtctgggctgccctcttagaagaggcgctctgctgctgctgtccatctacttctactacagcctgcctaacgctgtgggccctcctttcacatggatgctggctctgctgggcctgtctcaggctctgaatatcctgctggggctgaaaggcctggctcctgctgaaatctctgccgtgtgcgagaagggcaacttctctgtggctcacggcctggcctggtcctactacatcggatacctgagactgatcctgcctgagctgcaggccagaatcagaacctacaaccagcactacaacaacctgctgagaggcgctgtgtcccagagactgtacatcctgctgcctctggactgtggcgtgccagacaacctgtctatggccgatcctaacatccggtttctggacaagctgcctcagcagacaggcgatcacgccggcatcaaggacagagtgtacagcaacagcatctacgagctgctggaaaacggccagcgagccggaacatgcgtgctggaatatgccacacctctgcagaccctgttcgccatgagccagtactctcaggccggcttcagcagagaggacagactggaacaggccaagctgttctgcaggaccctcgaggacatcctggctgatgctccagagagccagaacaactgcagactgatcgcctaccaagagcctgccgacgacagcagctttagcctgagccaagaggtgctgagacacctgagacaagaagagaaagaggaagtcaccgtcggcagcctgaaaaccagcgccgtgcctagcaccagcaccatgtctcaagaacccgaactgctgatctccggcatggaaaagcccctgcctctgagaaccgacttcagctag

SEQ ID NO: 5 - protein, 379 aa, Artificial Sequence
  Synthetic construct, STING R284M mutant
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDMLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 6 - DNA, 1140 bp, Artificial Sequence
  Synthetic construct, STING R284M mutant coding seq
atgccccacagctctctgcaccctagcatcccttgtcctagaggacacggcgcccagaaagctgctctggttctgctgtctgcctgcctggttacactgtggggactgggagagcctcctgagcacacactgagatacctggtgctgcacctggcttctctgcagctgggactgctgctgaacggcgtgtgttctctggccgaggaactgagacacatccacagcagatacaggggcagctactggcggacagtcagagcttgtctgggctgccctcttagaagaggcgctctgctgctgctgtccatctacttctactacagcctgcctaacgctgtgggccctcctttcacatggatgctggctctgctgggcctgtctcaggctctgaatatcctgctggggctgaaaggcctggctcctgctgaaatctctgccgtgtgcgagaagggcaacttcaacgtggcacacggcctggcctggtcctactacatcggatacctgagactgatcctgcctgagctgcaggccagaatcagaacctacaaccagcactacaacaacctgctgagaggcgctgtgtcccagagactgtacatcctgctgcctctggactgtggcgtgccagacaacctgtctatggccgatcctaacatccggtttctggacaagctgcctcagcagacaggcgatcacgccggcatcaaggacagagtgtacagcaacagcatctacgagctgctggaaaacggccagcgagccggaacatgcgtgctggaatatgccacacctctgcagaccctgttcgccatgagccagtactctcaggccggcttcagccgcgaggatatgctggaacaggccaagctgttctgcaggaccctcgaggacatcctggctgatgctccagagagccagaacaactgcagactgatcgcctaccaagagcctgccgacgacagcagctttagcctgagccaagaggtgctgagacacctgagacaagaagagaaagaggaagtcaccgtcggcagcctgaaaaccagcgccgtgcctagcaccagcaccatgtctcaagaacccgaactgctgatctccggcatggaaaagcccctgcctctgagaaccgacttcagctag

SEQ ID NO: 7 - protein, 379 aa, Artificial Sequence
  Synthetic construct, STING N154S/R284M mutant
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFSVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDMLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 8 - DNA, 1140 bp, Artificial Sequence
  Synthetic construct, STING N154S/R284M mutant coding seq
atgccccacagctctctgcaccctagcatcccttgtcctagaggacacggcgcccagaaagctgctctggttctgctgtctgcctgcctggttacactgtggggactgggagagcctcctgagcacacactgagatacctggtgctgcacctggcttctctgcagctgggactgctgctgaacggcgtgtgttctctggccgaggaactgagacacatccacagcagatacaggggcagctactggcggacagtcagagcttgtctgggctgccctcttagaagaggcgctctgctgctgctgtccatctacttctactacagcctgcctaacgctgtgggccctcctttcacatggatgctggctctgctgggcctgtctcaggctctgaatatcctgctggggctgaaaggcctggctcctgctgaaatctctgccgtgtgcgagaagggcaacttctctgtggctcacggcctggcctggtcctactacatcggatacctgagactgatcctgcctgagctgcaggccagaatcagaacctacaaccagcactacaacaacctgctgagaggcgctgtgtcccagagactgtacatcctgctgcctctggactgtggcgtgccagacaacctgtctatggccgatcctaacatccggtttctggacaagctgcctcagcagacaggcgatcacgccggcatcaaggacagagtgtacagcaacagcatctacgagctgctggaaaacggccagcgagccggaacatgcgtgctggaatatgccacacctctgcagaccctgttcgccatgagccagtactctcaggccggcttcagccgcgaggatatgctggaacaggccaagctgttctgcaggaccctcgaggacatcctggctgatgctccagagagccagaacaactgcagactgatcgcctaccaagagcctgccgacgacagcagctttagcctgagccaagaggtgctgagacacctgagacaagaagagaaagaggaagtcaccgtcggcagcctgaaaaccagcgccgtgcctagcaccagcaccatgtctcaagaacccgaactgctgatctccggcatggaaaagcccctgcctctgagaaccgacttcagctag

SEQ ID NO: 9 - RNA, 1140 nt, Artificial Sequence
  Synthetic construct, WT STING coding seq_RNA
augccccacagcucucugcacccuagcaucccuuguccuagaggacacggcgcccagaaagcugcucugguucugcugucugccugccugguuacacuguggggacugggagagccuccugagcacacacugagauaccuggugcugcaccuggcuucucugcagcugggacugcugcugaacggcguguguucucuggccgaggaacugagacacauccacagcagauacaggggcagcuacuggcggacagucagagcuugucugggcugcccucuuagaagaggcgcucugcugcugcuguccaucuacuucuacuacagccugccuaacgcugugggcccuccuuucacauggaugcuggcucugcugggccugucucaggcucugaauauccugcuggggcugaaaggccuggcuccugcugaaaucucugccgugugcgagaagggcaacuucaacguggcacacggccuggccugguccuacuacaucggauaccugagacugauccugccugagcugcaggccagaaucagaaccuacaaccagcacuacaacaaccugcugagaggcgcugugucccagagacuguacauccugcugccucuggacuguggcgugccagacaaccugucuauggccgauccuaacauccgguuucuggacaagcugccucagcagacaggcgaucacgccggcaucaaggacagaguguacagcaacagcaucuacgagcugcuggaaaacggccagcgagccggaacaugcgugcuggaauaugccacaccucugcagacccuguucgccaugagccaguacucucaggccggcuucagcagagaggacagacuggaacaggccaagcuguucugcaggacccucgaggacauccuggcugaugcuccagagagccagaacaacugcagacugaucgccuaccaagagccugccgacgacagcagcuuuagccugagccaagaggugcugagacaccugagacaagaagagaaagaggaagucaccgucggcagccugaaaaccagcgccgugccuagcaccagcaccaugucucaagaacccgaacugcugaucuccggcauggaaaagccccugccucugagaaccgacuucagcuag

SEQ ID NO: 10 - RNA, 1140 nt, Artificial Sequence
  Synthetic construct, STING mutant  N154S coding sequence_RNA
augccccacagcucucugcacccuagcaucccuuguccuagaggacacggcgcccagaaagcugcucugguucugcugucugccugccugguuacacuguggggacugggagagccuccugagcacacacugagauaccuggugcugcaccuggcuucucugcagcugggacugcugcugaacggcguguguucucuggccgaggaacugagacacauccacagcagauacaggggcagcuacuggcggacagucagagcuugucugggcugcccucuuagaagaggcgcucugcugcugcuguccaucuacuucuacuacagccugccuaacgcugugggcccuccuuucacauggaugcuggcucugcugggccugucucaggcucugaauauccugcuggggcugaaaggccuggcuccugcugaaaucucugccgugugcgagaagggcaacuucucuguggcucacggccuggccugguccuacuacaucggauaccugagacugauccugccugagcugcaggccagaaucagaaccuacaaccagcacuacaacaaccugcugagaggcgcugugucccagagacuguacauccugcugccucuggacuguggcgugccagacaaccugucuauggccgauccuaacauccgguuucuggacaagcugccucagcagacaggcgaucacgccggcaucaaggacagaguguacagcaacagcaucuacgagcugcuggaaaacggccagcgagccggaacaugcgugcuggaauaugccacaccucugcagacccuguucgccaugagccaguacucucaggccggcuucagcagagaggacagacuggaacaggccaagcuguucugcaggacccucgaggacauccuggcugaugcuccagagagccagaacaacugcagacugaucgccuaccaagagccugccgacgacagcagcuuuagccugagccaagaggugcugagacaccugagacaagaagagaaagaggaagucaccgucggcagccugaaaaccagcgccgugccuagcaccagcaccaugucucaagaacccgaacugcugaucuccggcauggaaaagccccugccucugagaaccgacuucagcuag

SEQ ID NO: 11 - RNA, 1140 nt, Artificial Sequence
  Synthetic construct, STING mutant  R284M coding seq_RNA
augccccacagcucucugcacccuagcaucccuuguccuagaggacacggcgcccagaaagcugcucugguucugcugucugccugccugguuacacuguggggacugggagagccuccugagcacacacugagauaccuggugcugcaccuggcuucucugcagcugggacugcugcugaacggcguguguucucuggccgaggaacugagacacauccacagcagauacaggggcagcuacuggcggacagucagagcuugucugggcugcccucuuagaagaggcgcucugcugcugcuguccaucuacuucuacuacagccugccuaacgcugugggcccuccuuucacauggaugcuggcucugcugggccugucucaggcucugaauauccugcuggggcugaaaggccuggcuccugcugaaaucucugccgugugcgagaagggcaacuucaacguggcacacggccuggccugguccuacuacaucggauaccugagacugauccugccugagcugcaggccagaaucagaaccuacaaccagcacuacaacaaccugcugagaggcgcugugucccagagacuguacauccugcugccucuggacuguggcgugccagacaaccugucuauggccgauccuaacauccgguuucuggacaagcugccucagcagacaggcgaucacgccggcaucaaggacagaguguacagcaacagcaucuacgagcugcuggaaaacggccagcgagccggaacaugcgugcuggaauaugccacaccucugcagacccuguucgccaugagccaguacucucaggccggcuucagccgcgaggauaugcuggaacaggccaagcuguucugcaggacccucgaggacauccuggcugaugcuccagagagccagaacaacugcagacugaucgccuaccaagagccugccgacgacagcagcuuuagccugagccaagaggugcugagacaccugagacaagaagagaaagaggaagucaccgucggcagccugaaaaccagcgccgugccuagcaccagcaccaugucucaagaacccgaacugcugaucuccggcauggaaaagccccugccucugagaaccgacuucagcuag

SEQ ID NO: 12 - RNA, 1140 nt, Artificial Sequence
  Synthetic construct, STING mutant N154S/R284M coding seq_RNA
augccccacagcucucugcacccuagcaucccuuguccuagaggacacggcgcccagaaagcugcucugguucugcugucugccugccugguuacacuguggggacugggagagccuccugagcacacacugagauaccuggugcugcaccuggcuucucugcagcugggacugcugcugaacggcguguguucucuggccgaggaacugagacacauccacagcagauacaggggcagcuacuggcggacagucagagcuugucugggcugcccucuuagaagaggcgcucugcugcugcuguccaucuacuucuacuacagccugccuaacgcugugggcccuccuuucacauggaugcuggcucugcugggccugucucaggcucugaauauccugcuggggcugaaaggccuggcuccugcugaaaucucugccgugugcgagaagggcaacuucucuguggcucacggccuggccugguccuacuacaucggauaccugagacugauccugccugagcugcaggccagaaucagaaccuacaaccagcacuacaacaaccugcugagaggcgcugugucccagagacuguacauccugcugccucuggacuguggcgugccagacaaccugucuauggccgauccuaacauccgguuucuggacaagcugccucagcagacaggcgaucacgccggcaucaaggacagaguguacagcaacagcaucuacgagcugcuggaaaacggccagcgagccggaacaugcgugcuggaauaugccacaccucugcagacccuguucgccaugagccaguacucucaggccggcuucagccgcgaggauaugcuggaacaggccaagcuguucugcaggacccucgaggacauccuggcugaugcuccagagagccagaacaacugcagacugaucgccuaccaagagccugccgacgacagcagcuuuagccugagccaagaggugcugagacaccugagacaagaagagaaagaggaagucaccgucggcagccugaaaaccagcgccgugccuagcaccagcaccaugucucaagaacccgaacugcugaucuccggcauggaaaagccccugccucugagaaccgacuucagcuag

SEQ ID NO: 13 - protein, 148 aa, Artificial Sequence
  Synthetic construct, TLuc protein
MEAEAERGKLPGKKLPLEVLIELEANARKAGCTRGCLICLSKIKCTAKMKKYIPGRCADYGGDKKTGQAGIVGAIVDIPEISGFKEMEPMEQFIAQVDRCADCTTGCLKGLANVKCSDLLKKWLPGRCATFADKIQSEVDNIKGLAGD

SEQ ID NO: 14 - DNA, 447 bp, Artificial Sequence
  Synthetic construct, TLuc encoding seq_DNA
atggaagctgaagccgagcggggaaaactgcctggaaagaaactgccactggaggtcctgattgaactggaagcaaacgcaagaaaggccggctgcaccaggggctgcctgatctgtctgtctaagatcaagtgcacagccaagatgaagaagtacatcccaggcagatgtgccgactatggaggcgataagaagaccggacaggcaggaatcgtgggagcaatcgtggacatccccgagatcagcggcttcaaggagatggagcctatggagcagtttatcgcccaggtggaccggtgcgccgattgtaccacaggctgcctgaagggcctggccaacgtgaagtgttccgatctgctgaagaagtggctgcccggccgctgtgccacttttgccgataagattcagagcgaggtggataacatcaaggggctggctggggactaa

SEQ ID NO: 15 - RNA, 446 nt, Artificial Sequence
  Synthetic construct, TLuc encoding seq_RNA
auggaagcugaagccgagcggggaaaacugccuggaaagaaacugccacuggagguccugauugaacuggaagcaaacgcaagaaaggccggcugcaccaggggcugccugaucugucugucuaagaucaagugcacagccaagaugaagaaguacaucccaggcagaugugccgacuauggaggcgauaagaagaccggacaggcaggaaucgugggagcaaucguggacauccccgagaucagcggcuucaaggagauggagccuauggagcaguuuaucgcccagguggaccggugcgccgauuguaccacaggcugccugaagggccuggccaacgugaaguguuccgaucugcugaagaaguggcugcccggccgcugugccacuuuugccgauaagauucagagcgagguggauaacaucaaggggcuggcuggggacaa

SEQ ID NO: 16 - protein, 270 aa, Artificial Sequence
  Synthetic construct, STING FS protein
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFSVAHGLAWSYYIGYLRLILPELQARIRTYNQHYNNLLRGACPRDCTSCCLWTVACQTTCLWPILTSGFWTSCLSRQAITPASRTECTATASTSCWKTASEPEHACWNMPHLCRPCSP

SEQ ID NO: 17 - DNA, 813 bp, Artificial Sequence
  Synthetic construct, STING FS coding seq_DNA
atgccccacagctctctgcaccctagcatcccttgtcctagaggacacggcgcccagaaagctgctctggttctgctgtctgcctgcctggttacactgtggggactgggagagcctcctgagcacacactgagatacctggtgctgcacctggcttctctgcagctgggactgctgctgaacggcgtgtgttctctggccgaggaactgagacacatccacagcagatacaggggcagctactggcggacagtcagagcttgtctgggctgccctcttagaagaggcgctctgctgctgctgtccatctacttctactacagcctgcctaacgctgtgggccctcctttcacatggatgctggctctgctgggcctgtctcaggctctgaatatcctgctggggctgaaaggcctggctcctgctgaaatctctgccgtgtgcgagaagggcaacttctctgtggctcacggcctggcctggtcctactacatcggatacctgagactgatcctgcctgagctgcaggccagaatcagaacctacaaccagcactacaacaacctgctgagaggcgcgtgtcccagagactgtacatcctgctgcctctggactgtggcgtgccagacaacctgtctatggccgatcctaacatccggtttctggacaagctgcctcagcagacaggcgatcacgccggcatcaaggacagagtgtacagcaacagcatctacgagctgctggaaaacggccagcgagccggaacatgcgtgctggaatatgccacacctctgcagaccctgttcgccatga

SEQ ID NO: 18 - RNA, 813 nt, Artificial Sequence
  Synthetic construct, STING FS coding seq_RNA
augccccacagcucucugcacccuagcaucccuuguccuagaggacacggcgcccagaaagcugcucugguucugcugucugccugccugguuacacuguggggacugggagagccuccugagcacacacugagauaccuggugcugcaccuggcuucucugcagcugggacugcugcugaacggcguguguucucuggccgaggaacugagacacauccacagcagauacaggggcagcuacuggcggacagucagagcuugucugggcugcccucuuagaagaggcgcucugcugcugcuguccaucuacuucuacuacagccugccuaacgcugugggcccuccuuucacauggaugcuggcucugcugggccugucucaggcucugaauauccugcuggggcugaaaggccuggcuccugcugaaaucucugccgugugcgagaagggcaacuucucuguggcucacggccuggccugguccuacuacaucggauaccugagacugauccugccugagcugcaggccagaaucagaaccuacaaccagcacuacaacaaccugcugagaggcgcgugucccagagacuguacauccugcugccucuggacuguggcgugccagacaaccugucuauggccgauccuaacauccgguuucuggacaagcugccucagcagacaggcgaucacgccggcaucaaggacagaguguacagcaacagcaucuacgagcugcuggaaaacggccagcgagccggaacaugcgugcuggaauaugccacaccucugcagacccuguucgccauga

(19) United States
(12) Patent Application Publication
(10) Pub. No.: US 2021/0330600 A1
(43) Pub. Date: Oct. 28, 2021

(54) NANOPARTICLE COMPOSITIONS FOR EFFICIENT NUCLEIC ACID DELIVERY AND METHODS OF MAKING AND USING THE SAME

(71) Applicant: TIBA BIOTECH LLC, Cambridge, MA (US)

(72) Inventors: Poulami TALUKDER, Woburn, MA (US); Jasdave CHAHAL, Melrose, MA (US); Justine McPARTLAN, Chelsea, MA (US); Omar KHAN, Woburn, MA (US); Karl RUPING, Cambridge, MA (US)

(73) Assignee: TIBA BIOTECH LLC, Cambridge, MA (US)

(21) Appl. No.: 17/414,193

(22) PCT Filed: Dec. 19, 2019
(86) PCT No.: PCT/US2019/067402

§ 371 Date: Jun. 15, 2021

Related U.S. Application Data
(60) Provisional application No. 62/784,129, filed on Dec. 21, 2018.

(51) Int. Cl. A61K 9/51; A61K 39/39; A61K 49/18; A61K 49/12; A61K 38/17

(52) U.S. Cl. CPC A61K 9/5146; A61K 39/39; A61K 2039/53; A61K 49/124; A61K 38/1709; A61K 49/1818

(57) ABSTRACT

Nanoparticle compositions for delivery of nucleic acids to subjects including modified dendrimers comprising cores, one or more of homogeneous or heterogeneous intermediate and terminal layers, and therapeutic or immunogenic nucleic acid agents enclosed within nanop article compositions are described. Methods for treating or preventing diseases or conditions in a subject by administering the nanoparticle compositions that provide immune responses and synergistic therapeutic or preventive effects are provided.

### CROSS REFERENCE TO RELATED APPLICATION

This application claims the benefit of U.S. provisional application No. 62/784,129, filed Dec. 21, 2018, which is incorporated by reference as if fully set forth.

The sequence listing electronically filed with this application titled “Sequence Listing,” which was created on Dec. 19, 2019 and had a size of 34,617 bytes is incorporated by reference herein as if fully set forth.

### FIELD OF INVENTION

The disclosure relates to nanoparticle compositions for efficient delivery of nucleic acids to a subject for treating or preventing diseases and/or disorders, and more specifically, for nanoparticle compositions comprising nucleic acids encompassed within homogeneous or heterogeneous modified dendrimers. The disclosure also relates to methods of formulating the nanop article compositions and methods of treating diseases and/or disorders in the subjects with such nanoparticle compositions.

### BACKGROUND

Despite progress made in design and/or production of nucleic acid vaccines and therapeutics, significant challenges remain in their delivery to the patients. Attempts were made to administer pure DNA or RNA molecules directly to the target tissues (e.g., lymph nodes) with various degrees of success (Kreiter et al., Cancer Res. 70, 9031-9040 (2010), which is incorporated herein by reference as if fully set forth). Purified mRNAs were found to be particularly unstable due to degradation by hydroxyl radicals and endonucleases. Large self-replicating RNA molecules (RepRNA; 12-15 kb) were recently reported for successful delivery of vaccine antigen payload to cells. The major limitations with RepRNA as a delivery vehicle, however, include RNase-sensitivity and inefficient uptake by dendritic cells (DCs)—the pre-requisite for efficacious vaccine design. The limited translocation across cell membranes additionally limits the application of RNA-based vaccines and therapeutics. One of the approaches to deliver nucleic acid vaccines utilizes dendrimers, synthetic spherical tree-like branched molecules. Poly(amidoamine) (PAMAM) dendrimers, in particular, have been used due to their multivalency, biocompatibility, and tolerability in humans.

The general application of dendrimers, including PAMAM, is still limited due to cytotoxicity issues. Dendrimer cytotoxicity depends on the generation, the number of surface groups, and the nature of terminal moieties (anionic, neutral, or cationic). While less cytotoxic, a low generation dendrimer has fewer surface primary amines and less rigid surface structure due to its smaller size and paucity of branching. Such dendrimers do not efficiently complex with nucleic acids. A higher generation dendrimer has more surface primary amines that form a rigidly spherical surface exhibiting a high density of charges and more efficiently complexes with nucleic acids. For example, it has been reported that the generation 1 (G1) PAMAM does not complex nucleic acids because of low positive charge density whereas the high generation PAMAM is able to complex with nucleic acids (Jensen et al., Int J Pharm, 2011, 416, pp. 410-418; and Palmerston et al., Molecules, 2017, 22, 1401, both of which are incorporated herein by reference as if fully set forth).

In order to decrease the cytotoxicity, scientists started to introduce different chemical modifications on the periphery of the dendrimer. (Janaszewske et al. 2019, Biomolecules 2019, 9, 330, which is incorporated herein by reference as if fully set forth). Modified dendrimer carriers containing alkyl substitutions, also referred to as alkylated dendrimers, have been reported to complex with nucleic acids to form nanoparticles (US 2017/0079916 A1, published Mar. 23, 2017, which is incorporated herein by reference as if fully set forth).

Chemical modifications of the periphery of dendrimers determine their biological activity, physiochemical properties and biocompatibility. The ideal nucleic acid delivery vehicle should be biocompatible to prevent bioaccumulation and subsequent cytotoxicity. Delivery molecules should also be capable of self-assembly, colloidal stability, thermostability, endonuclease protection, controlled release and/or hydroxide ion- scavenging.

### SUMMARY

In an aspect, the invention relates to a nanoparticle composition comprising a modified dendrimer and a therapeutic or immunogenic nucleic acid agent enclosed within the nanoparticle composition. The modified dendrimer comprises a plurality of terminal amine groups substituted with fatty acids or derivatives thereof.

In an aspect, the invention relates to a nanop article composition comprising a modified dendrimer and a therapeutic or immunogenic nucleic acid agent enclosed within the nanoparticle composition. The modified dendrimer comprises a core, a plurality of heterogeneous intermediate layers, and a terminal layer. The plurality of heterogeneous intermediate layers comprises at least one layer modified for endosomal escape, at least one layer modified for hydroxide ion- scavenging, or both.

In an aspect, the invention relates to a method for treating or preventing a disease or condition in a subject. The method comprises providing any one of the nanoparticle compositions described herein and administering a therapeutically effective amount of the nanoparticle composition to a subject.

In an aspect, the invention relates to a method of manufacturing a nanoparticle composition capable of altering the rate of the nucleic acid release in cytoplasm of the cell comprising formulating the nanop article composition at pH ranging from 3.0 to 6.5.

### BRIEF DESCRIPTION OF THE DRAWINGS

The following detailed description of preferred embodiments of the present invention will be better understood when read in conjunction with the appended drawings. For the purpose of illustrating the invention, particular embodiments are shown in the drawings. It is understood, however, that the invention is not limited to the precise arrangements and instrumentalities shown. In the drawings:

FIGS. 1A-1B illustrate modified dendrimers that include fatty acids in the terminal layer. FIG. 1A is a schematic drawing of the generation 1 modified PAMAM dendrimer and fatty acid moieties (R) that can be used for modification. FIG. 1B illustrates synthesis of the PAMAM dendrimers modified with a fatty acid, linoleic acid.

FIG. 2 illustrates a process for preparing a nanoparticle composition designed for improved self-assembly and biocompatibility.

FIG. 3 illustrates illustrates particle size distribution of nanoparticles generated by mixture of PG1-linoleic acid modified dendrimer and SEAP replicon.

FIG. 4 illustrates a photograph of the agarose gel showing the binding of the modified dendrimer with RNA.

FIG. 5 illustrates the SEAP expression of nanoparticle formulations using PG1-oleic acids and PG1-linoleic acids based on absorbance at OD₆₃₅ (compared to the negative control “No transfection”).

FIG. 6 illustrates the process of preparing a synthetic vaccine that includes a modified dendrimer, 1,2 dimyristoyl-sn-glycero-3-phosphoethanlomine-N[methoxy(polyethylene glycol)-2000], and replicons.

FIGS. 7A-7C illustrate the effect of formulation pH on the nanoparticles' stability and the replicon release time.

FIG. 7A illustrates the SEAP colorimetric signal for the replicon mRNA expressing SEAP that were synthesized and formulated into modified dendrimer nanop articles at a pH of 3.0 or 5.0.

FIG. 7B illustrates the SEAP colorimetric signal for the replicon mRNA expressing SEAP synthesized and formulated into modified dendrimer nanoparticles at a pH of 3.0 or 5.0 and collected from mice five days after administration.

FIG. 7C illustrates the effect of increasing the pH during the nanoparticle manufacturing process on the in vivo performance of the nanoparticles.

FIGS. 8A-8E illustrate the effect of formulation pH on vaccine performance in vivo. FIG. 8A illustrates steps of the ELISPOT test used to assess the T cell response following vaccination with nanoparticles formulated at pH 3.0, 5.0 and 6.0 and containing replicons expressing Ebola GP. For this test, animals (n=5) were vaccinated with the nanoparticles. FIGS. 8B-8E illustrate the ELISPOT test results for unimmunized control cells (FIG. 8B), for nanoparticles formulated at pH 3.0 (FIG. 8C), for nanoparticles formulated at pH 5.0 (FIG. 8D), and for nanoparticles formulated at pH 6.0 (FIG. 8E).

FIG. 9 illustrates a structure of a cyclic guanosine monophosphate-adenosine monophosphate (2′3′-cGAMP), an exemplary STING activator, in which H groups of the primary amine (NH₂) are substituted with the hydrophobic R functional groups.

FIG. 10 illustrates structures of the modified dendrimers comprising the cores incorporating stable isotopes of nitrogen (¹⁵N; top structures) and carbon (¹³C; bottom structures).

FIG. 11 illustrates the effect of incorporation of amphiphilic PEG molecules on the particle size and aggregation ability of the nanoparticle composition.

FIG. 12 illustrates the effect of incorporation of amphiphilic PEG molecules on the diameter and concentration of the particle is the nanop article composition.

FIG. 13 illustrates the effect of incorporation of amphiphilic PEG molecules on the number of RNA molecules per nanoparticle.

FIG. 14 illustrates the effect of incorporation of amphiphilic PEG molecules on the ability to increase the degree of the electrostatic repulsion between nanoparticles and their dispersion.

FIG. 15 illustrates exemplary sulfone-based drugs exemplary sulfonylurea drugs acetohexamide, chlorprop amide, tolbutamide, glibenclamide, glipizide, glimepiride, and gliclazide that can be included in the nanoparticle compositions according to the embodiments herein.

FIG. 16 illustrates that the stability of the PG1.C12 modified dendrimer nanop articles containing acetohexamide as assessed by the particle size distribution measured by dynamic light scattering following production (Day 0; solid line) and 12 days after formulation and storage at 4° C. (Day 12; dashed line).

FIG. 17 illustrates the quantified data on the IFN Type I response in the reporter cells transfected with nucleic acids encoding STING proteins.

FIG. 18 illustrates the quantified data on the IFN Type II response in the reporter cells transfected with nucleic acids encoding STING proteins.

FIG. 19 illustrates Western blot analysis of the expression level of STING (above what is naturally present) in each transfection experiment.

FIG. 20 illustrates the Western blot analysis of the expression level of STING (above what is naturally present) in each transfection experiment.

FIGS. 21A-21D illustrate gene expression and activation of the IFN Type I response in the B16 type I reporter cells (B16 Blue IFN I cells, Invivogen) following treatment with the modified-dendrimer (PG1.C12 in FIGS. 22A-22B or PG1.C15 in FIGS. 22C-22D)/PEG-lipid formulated TLuc mRNA in combination with mRNA encoding either STING protein inactivated by a frame-shift mutation (TLuc+STING^{FS}mRNA) or constitutively active STING (double-mutant N154S/R284M; TLuc+STING mRNA).

FIG. 21A illustrates intensity of IFN type I signaling in B16 type I reporter cells (B16 Blue IFN I cells, Invivogen) treated with the modified-dendrimer PG1.C12 formulations TLuc+STING^{FS}mRNA, TLuc+STING mRNA compared to “No treatment” control.

FIG. 21B illustrates the intensity of TLuc gene expression in B16 type I reporter cells (B16 Blue IFN I cells, Invivogen) treated with the modified-dendrimer PG1.C12 formulations TLuc +STING^{FS}mRNA, TLuc+STING mRNA compared to “No treatment” control.

FIG. 21C illustrates intensity of IFN type I signaling in B16 type I reporter cells (B16 Blue IFN I cells, Invivogen) treated with the modified-dendrimer PG1.C15 formulations TLuc+STING^{FS}mRNA, TLuc+STING mRNA compared to “No treatment” control.

FIG. 21D illustrates intensity of TLuc gene expression in B16 type I reporter cells (B16 Blue IFN I cells, Invivogen) treated with the modified-dendrimer PG1.C15 formulations TLuc +STING^{FS}mRNA, TLuc+STING mRNA compared to “No treatment” control.

FIGS. 22A-22C illustrate gene expression and activation of the IFN Type I response in the B16 type I reporter cells (B16 Blue IFN I cells, Invivogen) following treatment with the PG1.C15 CDN nanoparticles.

FIG. 22A illustrates intensity of the IFN Type I stimulation activity in the B16 type I reporter cells (B16 Blue IFN I cells, Invivogen) following treatment with the dialyzed modified dendrimer-based RNA nanoparticles (PG1.C15 CDN) formulated with a cyclic dinucleotide (CDN) and normalized to the activity of corresponding pre-dialyzed samples, and CDN alone.

FIGS. 22B illustrates intensity of IFN type I signaling in B16 type I reporter cells (B16 Blue IFN I cells, Invivogen) treated with modified-dendrimer/PEG-lipid formulated (PG1.C15; Modified Dendrimer formulated) or unformulated (No Dendrimer formulation) TLuc mRNA and CDN.

FIG. 22C illustrates intensity of TLuc gene expression in B16 type I reporter cells (B16 Blue IFN I cells, Invivogen) treated with modified-dendrimer/PEG-lipid formulated (PG1.C15; (No Dendrimer formulation) or unformulated (No Dendrimer) TLuc mRNA and CDN.

FIG. 23 illustrates the reaction to add alkyl groups to the terminal layer of the PAMAM G1 dendrimer via the primary and secondary amines with the terminal epoxide on an alkyl chain

FIG. 24 illustrates the thin layer chromatography plate showing the multiple modified dendrimers produced during a single reaction, each containing a different degree of substitution DZₙ.

FIG. 25 illustrates molecular structures of modified dendrimers comprising an amine-containing core (top) and one (middle top), two (middle bottom), or three (bottom) layers.

FIGS. 26A-26C illustrate molecular structures of the modified dendrimers comprising 1,2-diaminoethane (left) and 1,4-diaminobutane (right) cores, and 2 (FIG. 26A), 3 (FIG. 26B), or 4 (FIG. 26C) layers. In the figure, R is represented by the formula CₙH₂ₙ₊₁.

FIG. 27 illustrates molecular structures of the three-layer modified dendrimers comprising 1,2-diaminoethane (left) and 1,4-diaminobutane (right) cores. In the figure, R is C₁₃H₂₇.

FIGS. 28A-28G illustrate synthesis of modified dendrimers. FIG. 28A illustrates the synthesis steps of a three-layer modified dendrimer. FIGS. 28B-28C illustrate R groups that can be used as cores in modified dendrimers. FIG. 28D illustrates R groups used for synthesis steps I and II. FIGS. 28E-28G illustrate exemplary reactants and R groups for step III of the synthesis process.

FIG. 29 illustrates synthesis of an exemplary three layer modified dendrimer.

FIG. 30 illustrates modified dendrimers with high level of substitutions (tertiary only; top), low level of substitutions (secondary only; middle), and intermediate level of substitutions (tertiary and secondary; bottom).

FIG. 31 illustrates the RNA payload efficacy in vivo for high substitution nanoparticle, low substitution nanoparticle and blend of low, intermediate and high substitution nanoparticles, and correlation of the efficacy with diameters of the nanoparticles.

FIG. 32 illustrates exemplary dendrimers modified for hydroxide ion-scavenging and incorporating secondary and tertiary amines in their terminal (last) layers.

FIGS. 33A-33C illustrate particle size distribution of nanoparticles formed by modified and unmodified dendrimers based on dynamic light scattering (DLS) measurement of nanoparticles.

FIG. 33A illustrates particle size distribution of nanoparticles generated by mixture of PG1.C15 modified dendrimer and SEAP mRNA.

FIG. 33B illustrates particle size distribution of nanoparticles generated by mixture of PG1.C12 modified dendrimer and SEAP mRNA.

FIG. 33C illustrates particle size distribution of the mixture of unmodified PAMAM dendrimer and SEAP mRNA.

FIGS. 34A-34C illustrate molecular structures for the PG1.C15 (PAMAM-G1-EDA_C15) modified dendrimer (FIG. 34A), C12-200 (FIG. 34B) and 7C1 (FIG. 34C).

FIG. 35 illustrated the uptake efficiency of nanoparticles containing AlexaFluor 647-labelled RNA in human neural stem cells (NSCs) after a 3 hour treatment. RNA dose was 40 nmol. N=12 and error bars are±S.E.M.

FIG. 36 illustrates the transfer efficiencies of the nanop articles to glioblastoma (GBM) cells calculated as the percentage of the glioblastoma (GBM) cells containing AlexaFluor 647-labelled nanop articles that were recycled out of co-cultured human neuronal stem cells (NSCs). White bars are the 24 h time point, and grey bars are 72 h. N=12 and error bars are±S.E.M.

### DETAILED DESCRIPTION OF THE PREFERRED EMBODIMENTS

Certain terminology is used in the following description for convenience only and is not limiting.

The “nanoparticle composition” refers to a composition that includes a modified dendrimer and a nucleic acid payload molecule enclosed within the composition. The term “modified dendrimer” refers to one or more modified dendrimer molecules included in the nanoparticle composition.

The term “substitute” refers to the ability to change one functional group, or a moiety included therein, for another functional group on a molecule provided that the valency of all atoms is maintained. The substituted group is interchangeably referred herein as “substitution” or “substituent.” When more than one position in any given structure in substituted with more than one substituent selected from a specified group, the substituent may be either the same or different at every position.

The term “amine” refers to the NH₂ group and also refers to a nitrogen containing group derived from ammonia by the replacement of one or more hydrogen atoms by organic functional group. For example, the term “alkylamine” refers to an amine with an alkyl substituent group.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

In an embodiment, a modified dendrimer is provided. As used herein, the term “modified dendrimer” refers to a branched structure having a core, a plurality of homogenous or heterogeneous intermediate (interior) branched layers and a terminal (exterior) layer. The modified dendrimer may be formed by reacting a core to build symmetrical or asymmetrical branches of layers protruding or radiating from the core. The modified dendrimer may form concentric intermediate layers, with each layer increasing the molecular mass and the number and variety of functional groups at the end of each layer. The molecular weight of the modified dendrimer may range from 500 to 80,000 g/mole and the number of functional groups may range significantly.

In an embodiment, the modified dendrimer may be represented by the formula GᵢZₙ (I), where Gᵢ represents a layer or generation, wherein “i” is a generation number, Zₙ represents a functional group comprising reactive sites, wherein “n” is a number of reactive sites, and may be equal to or greater than 1. When “i”=t, Gt (or generation t) represents the terminal layer of the modified dendrimer. When “i”=c, G_{c} represents the core of the modified dendrimer.

Accordingly, in formula G_{c}Zₙ applicable to the core, Zₙ represents any polyvalent organic molecule having a functional group or groups with the (n) number of reactive sites to which the successive layer of the modified dendrimer can be attached. The number of reactive sites (n) on the core determines n-directionality of the modified dendrimer, and limits the number of units that can be added to form next layer. The core may contain one or more carboxylic acid groups, hydroxyl groups or amines as reactive sites. For example, if the core contains only one reactive site in the form of one carboxylic acid group or one hydroxyl group, it would lead to a 1→4 extension motif. If the core contains a primary amine, the amine nitrogen would then be divalent, leading to a 1→2 branching motif. If the core contains two primary amines, it would lead to a 1→4 branch extension. As used herein, the terms “primary, secondary, and tertiary amines” refer to nitrogens bound to one, two and three carbons, respectively. Secondary amines included in the core may also contain reactive sites and may define directionality of the modified dendrimers. Depending on the number of functional groups (Z) that can be linked to intermediate layers, the core may be uni-directional, bi-directional, tri-directional, tetra-directional, penta-directional, hexa-directional, or may have a greater directionality value. For example, the uni-directional core may be benzoic acid. The bi-directional core may be N¹, N³-dimethylpropane-1,3-diamine. The tri-directional core may be trimesic acid, or trimesoyl chloride. The tetra-directional core may be ethane-1,2-diamine, butane-1,4-diamine, 2,2′-(ethane-1,2-diylbis(oxy)bis(ethan-1-amine), 3-ureidopropanoic acid, or pentaerythritol. The penta-directional core may be N¹-(2-aminoethyl)propane-1,3-diamine or N¹-(2-(4-(2-aminoethyl)piperazin-1-yl)ethyl)ethane-1,2-diamine. The hexa-directional may be inositol or N¹, N¹-bis(2-aminoethyl)ethane-1,2-diamine.

The core may comprise —NH₂, —OH, —COOH, or —COCl groups that can be linked to intermediate layers.

The core comprising —NH₂, group may be, but is not limited to, ethylenediamine-, diaminobutane-, ethane-, butane-, N¹-(2-aminoethyl) ethane-, N¹-(2-aminoethyl)propane-, N³-dimethylpropane-, N¹,N¹′-(ethane-1,2-diyl)bis(ethane-), N¹-(2-(4-(2-aminoethyl)piperazin-1-yl)ethyl)ethane-1,2-diaminecyclohexane-, N¹-(2-(4-(2-aminoethyl)piperazin-1-yl)ethyl)ethane-1,2-diaminecyclohexane-, poly(ethylene)-, polyethylene-imine, urea, thiourea, hydrazinecarbothioamide, hydrazine carbothiohydrazide, or 3-ureidopropanoic acid. The core comprising —OH group may be pentaerythritol or inositol. The core comprising —COOH group may be benzoic acid. The core comprising —COCl group may be trimesoyl chloride. The core may comprise other functional groups that can be used for the same purpose. The core may comprise different functional groups as the terminal residues which can link the core with the subsequent intermediate layer. For example, the core may be 2,2′-(ethane-1,2-diylbis(azanediyl)bis(ethan-1-ol) and may comprise OH and NH groups which can be used for connecting the core with the subsequent layer.

The structure of the core may influence the number of functional groups on the surface, amine and/or charge density, diameter, and flexibility of the resultant modified dendrimer which may modulate their physicochemical properties, their interaction with nucleic acid, and gene transfer activity. A non-limiting example of the core may be ethyl diamine. The modified dendrimer comprising ethyl diamine with two methylene groups between amines may have a more rigid molecular structure compared to the modified dendrimer having a more flexible core, for example, butane diamine. The differences in flexibility of the modified dendrimer may influence the way they interact with nucleic acids and, therefore, affect the stability and transfection efficiency of the nanop article composition comprising the modified dendrimers.

In an embodiment, the core may mitigate challenges of intermediate products during synthesis of the modified dendrimers, and thus, may increase the overall manufacturing efficiency of the modified dendrimers as a delivery tool. For example, the core may comprise the UV active moiety such as trimesoyl chloride or benzoic acid.

In an embodiment, the core may be an ionizable core, and may be positively charged at low pH values.

In an embodiment, the core may be traceable and may contain stable isotopes. The stable isotopes may be ¹⁵N, ¹³C or both. The traceable core may be, but is not limited to, ethane-1,2-diamine-¹⁵N₂; ethane-1,2-diamine-1,2-¹³C₂; butane-1,2-diamine-¹⁵N₂; or butane-1,4-diamine-1,4-¹³C₂.

In an embodiment, the modified dendrimers may comprise a plurality of intermediate layers. The plurality of intermediate layers may be one to ten successive layers, and each layer may be represented by formula I (GᵢZₙ), wherein “i” is any integer that is equal to or greater than 1 and equal to or lesser than 10. Each intermediate layer may be formed by functional groups that include amines, and may be linked to the core or to the preceding layer through H substitutions in the amines. The plurality of intermediate layers may be heterogeneous intermediate layers. The plurality of intermediate layers may be homogeneous intermediate layers. In the homogeneous intermediate layers, the functional groups of all intermediate layers may be similar. In the heterogeneous intermediate layers, the functional groups of at least one intermediate layer may differ from the functional groups of other intermediate layers. In an embodiment, the plurality of the intermediate layers of the modified dendrimer may comprise other functional groups. The plurality of intermediate layers of the modified dendrimer may comprise functional groups that are similar for each layer except one layer. The plurality of intermediate layers may have functional groups that differ for each or for some layers. The functional group may be selected from saturated or unsaturated alkyl groups. As used herein, “alkyl” refers to the saturated or unsaturated aliphatic groups, including straight-chain alkyl, alkenyl, or alkynyl groups; cycloalkyl, cycloalkenyl, or cycloalkynyl (alicyclic) groups; alkyl substituted cycloalkyl, cycloalkenyl, or cycloalkynyl groups; cycloalkyl substituted alkyl, alkenyl, or alkynyl groups; branched-chain alkyl, alkenyl, or alkynyl groups; and straight-chain alkyl groups containing saturated or unsaturated alkyl branches. The alkyl groups in the intermediate layers may also contain one or more groups including, but not limited to, halogen, hydroxy, amino, thio, ether, ester, carboxy, oxo, or aldehyde groups.

Different layers of the plurality of intermediate layers may perform different functions. For example, one layer may primarily serve to scavenge certain ions in the nanoparticle diluent, one layer may bind nucleic acids, one layer may be modified for endosomal escape and another layer may provide self-assembly properties. Towards this design, the modified dendrimers may include hydroxide ion-scavenging layers. The term “hydroxide ion-scavenging” means absorbing, consuming, or reducing the amount of hydroxide ions from a given environment. Each layer of the modified dendrimers described herein may include one or more hydroxide ion-scavenging groups. The hydroxide ion-scavenging group(s) is typically a group capable of neutralizing hydroxide ions. To achieve the desired hydroxide ion-scavenging properties, a sufficient number of hydroxide ion-scavenging groups may be present in the modified dendrimers to achieve a suitable level of hydroxide ion-scavenging for a suitable length of time. Modified dendrimers may exhibit a high degree of hydroxide ion-scavenging group functionality. The modified dendrimer may include, for example, 2 or more hydroxide ion-scavenging groups, 5 or more hydroxide ion-scavenging groups, 10 or more hydroxide ion-scavenging groups, 20 or more hydroxide ion-scavenging groups, or 100 or more hydroxide ion-scavenging groups. Any suitable group capable of scavenging hydroxide ions may be employed at any suitable location within the multiple layers of modified dendrimers. The one or more hydroxide ion-scavenging group may be an acidic group. The one or more hydroxide ion-scavenging groups may be derived from proton donor(s), such as carboxylic acids, benzoic acid and propionic acid.

The modified dendrimers may include a layer modified to provide an endosomal escape of the nanoparticle upon entering a cell. The layer modified for endosomal escape may include functional groups having endosomolytic properties, i.e., promoting the lysis of and/or transport of the nanoparticles described herein, or its components, from the cellular compartments, such as the endosome, lysosome, endoplasmic reticulum (ER), golgi apparatus, microtubule, peroxisome, or other vesicular bodies within the cell, to the cytoplasm of the cell. The layer modified for endosomal escape may comprise a polyfluorocarbon. The polyfluorocarbon may comprise at least one moiety selected from the group consisting of nonafluoropentyl, tridecafluoroheptyl and heptadecafluorononyl groups.

In an embodiment the layer modified for endosomal escape may comprise unsaturated alkyl groups as described herein. In turn, the unsaturated alkyl groups may be further substituted with one or more groups including, but not limited to, halogen, hydroxy, amino, thio, ether, ester, carboxy, oxo, and aldehyde groups. The alkyl groups may also contain one or more heteroatoms. (Oliveira et al., 2007, Int J Pharm. 2007 Mar. 1; 331(2):211-4, which is incorporated herein by reference as if fully set forth),

The layer modified for endosomal escape may be modified with at least one functional group comprising an amine, or multiple amines. The amine(s) may be primary, secondary or tertiary amine(s) The nitrogens included in the amines may enhance endosomal escape by amplifying or accelerating the proton-pump effect in endosomes (Van Dyke, 1996, Subcell Biochem., 27:331-60, which is incorporated herein by reference as if fully set forth). Endosomes are acidified by a family of unique proton pumps, termed the vacuolar H(+)-ATPases. The electrogenic vacuolar H(+)-ATPase is responsible for generating electrical and chemical gradients across organelle membranes with the magnitude of these gradients ultimately determined by both proton pump regulatory mechanisms and, more importantly, associated ion and organic solute transporters located in vesicle membranes. This vacuolar proton pump acidifies the vesicle interior.

As used herein, the proton-pump effect refers to the process of increasing the concentration of protons within the endosome. As aqueous H+ ions are pumped into the endosome to acidify it (lower pH) the amines in the modified dendrimers that make up the nanop article may become protonated, consuming the protons and preventing a pH drop and endosome acidification (buffering effect). Thus, more aqueous proton solution must be pumped into the endosome to overcome this buffering effect. The volume of the endosome may swells, causing it to rupture. Because this amplified and accelerated proton pump effect occurs fast, it may enhance endosomal escape of the modified dendrimer.

In an embodiment, the layer modified for endosomal escape may contain one or more of imidazoles, poly or oligoimidazoles, linear or branched polyethyleneimines (PEIs), linear or branched polyamines, e.g. spermine, cationic linear or branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural or synthetic fusogenic lipids, natural or synthetic cationic lipids. The layer modified for endosomal escape may comprise a polyfluorocarbon. The polyfluorocarbon may comprise at least one moiety selected from the group consisting of nonafluoropentyl, tridecafluoroheptyl and heptadecafluorononyl groups.

In an embodiment, the layer modified for endosomal escape may be more than one layer. The layer modified for endosomal escape may include all layers of the modified dendrimer.

Alternatively, one or more of the molecules used for modifying layers of the dendrimer for endosomal escape as described herein may be added separately to the modified dendrimer or mixture of the modified dendrimer during self-assembly of nanop articles.

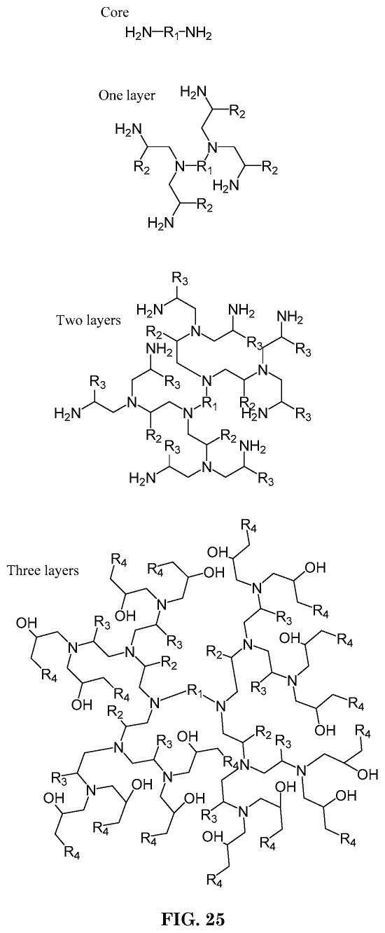

FIG. 25 illustrates molecular structures of modified dendrimers. As shown in this figure, the core contains amines linked through R₁ group. In a two layer modified dendrimer, the core amines are substituted with additional amine containing moieties also containing R₂ or R₃ groups. In a three layer modified dendrimer, the amines of the second layer are also substituted with amine containing moieties containing R₄ groups. Acidic groups may be added to any layer as R groups, to scavenge and inactivate hydroxide ions to prevent autocatalytic degradation of replicon payloads.

In an embodiment, the modified dendrimer may comprise a terminal layer. The terminal layer of the modified dendrimer may be represented by the formula GₜZₙ (I), where Gₜ refers to a terminal layer, Zₙ represents a functional group comprising reactive sites, wherein “n” is a number of reactive sites, and may be equal to or greater than 1. The modified dendrimer may be designed for superior self-assembly, colloidal stability, thermostability, endonuclease protection, controlled release and ion scavenging, the properties useful, for example, for optimal vaccine delivery.

In an embodiment, the terminal layer of the dendrimer may be modified with fatty acid substitutions for superior self-assembly. The fatty acids may be, but are not limited to, arachidonic acid, oleic acid, eicosapentanoic acid, lauric acid, caprylic acid, capric acid, myristic acid, palmitic acid, stearic acid, linoleic acid, linolenic acid, or derivatives thereof. The fatty acids may be any fatty acids with C₄-C₂₈ chains. The fatty acids derivatives may be, but are not limited to, fatty acid esters, acyl halides or anhydrides.

The terminal layer of the dendrimer may be modified to comprise hydrogen, methyl, ethyl, propyl, butyl, pentyl, hexyl, octyl, decyl, dodecyl, tridecyl, tetradecyl, pentadecyl, hexadecyl, octadecyl, but-3-en-1-yl, oct-7-en-1-yl, 2,2,3,3,4,4,5,5,5-nonafluoropentyl, 2,2,3,3,4,4,5,5,6,6,7,7,7-tridecafluoroheptyl, 2,2,3,3,4,4,5,5,6,6,7,7,8,8,9,9,9-heptadecafluorononyl, 12-tridecenyl, 14-pentadecenyl, 17-octadecenyl, oleyl, linoleyl, or arachidoneyl group.

The terminal layer of the modified dendrimer may be modified by contacting with a functional reagent selected from the group consisting of: oxirane, carboxylic acid, fluorophenyl ester, anhydride, isocyanate, isothiocyanate, aldehyde or carbonate. The functional reagent may have saturated or unsaturated alkyl or perfluoroalkyl substitution (C₁-C₂₀ chains). The functional reagent may be but is not limited to oxirane, 2-methyloxirane, 2-ethyloxirane, 2-propyloxirane, 2-butyloxirane, 2-pentyloxirane, 2-hexyloxirane, 2-octyloxirane, 2-decyloxirane, 2-dodecyloxirane, 2-tridecyloxirane, 2-tetradecyloxirane, 2-pentadecyloxirane, 2-octadecyloxirane, 2-(but-3-en-1-yl)oxirane, 2-(oct-3-en-yl)oxirane, 2-(2,2,3,3,4,4,5,5,5-nonafluoropentyl)oxirane, 2-(2,2,3,3,4,4,5,5,6,6,7,7,7-tridecafluoroheptyl) oxirane, 2-(2,2,3,3,4,4,5,5,6,6,7,7,8,8,9,9,9-heptadecafluorononyloxirane, stearic acid, palmitic acid, myristic acid, 16-heptadecenoic acid, 14-pentadecenoic acid, 12-tridecenoic acid, linolenic acid or linoleic acid, and derivatives thereof.

To prevent nanoparticle recycling, the terminal layer of the dendrimer may be modified by substitutions with unsaturated alkyl groups. The unsaturated alkyl groups may be more fluid, have a lower crystallization temperature compared to the saturated alkyl groups, and thus, may have the ability to morphologically change nanop articles containing the dendrimer into a fusogenic form when they interact with phospholipid bilayers of the cell membrane and to rupture the endosome. Thus, the nanoparticles may become restricted and may remain inside the cells at the site of injection only and may not be trafficked elsewhere.

In an embodiment, the dendrimer may be modified to have terminal layers comprised of lower molecular weight hydrophobic moieties, as compared to alkyl chains. The lower molecular weight hydrophobic moieties may be fluorocarbons. This type of modified dendrimers may lack steric hindrance that characterizes alkylated dendrimers. The fluorocarbon groups may assist with self-assembly into nanoparticles, but need shorter lengths to create hydrophobicity compared to functional groups of alkylated dendrimers. The modification of the dendrimers with fluorocarbons may reduce steric hindrance while reducing the molecular weight of the overall molecule, as compared to alkanes. Smaller three-layer modified dendrimers may have approximate molecular weights of 502.84, and larger multilayer modified dendrimers may have molecular weights in excess of 5000.

In an embodiment, the modified dendrimer may be a homogeneous dendrimer. As used herein, the term “homogeneous” dendrimer refers to dendrimers formed by layers with similar or identical functional groups. Examples of homogeneous dendrimer include, but are not limited to, poly(amidoamine) (PAMAM), polypropyleneimine (PPI), poly(ethyleneimine) (PEI), or polypropylamine (POPAM).

The homogeneous dendrimer may be poly(amido-amine) (PAMAM) dendrimer. PAMAM dendrimers may be G₀ to G₁₀ generation dendrimers. PAMAM dendrimer may be but is not limited to, G₀ PAMAM dendrimers, G₁ PAMAM dendrimers, G₂ PAMAM dendrimers, G₃ PAMAM dendrimers, G₄ PAMAM dendrimers, G₅ PAMAM dendrimers, G₆ PAMAM dendrimers, G₇ PAMAM dendrimers, G₈ PAMAM dendrimers, G₉ PAMAM dendrimers, or G₁₀ PAMAM dendrimers. For improved biocompatibility and low cytotoxicity, PAMAM dendrimers may be G₀ to G₂ generation dendrimers. An exemplary molecular structure of G₀ PAMAM dendrimer of formula (II) is shown below.

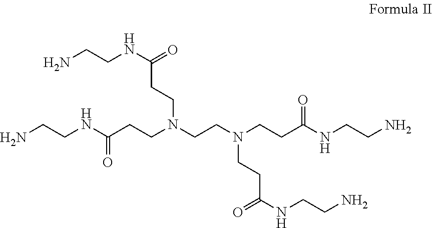 [SMILES: NCCNC(=O)CCN(CCC(=O)NCCN)CCN(CCC(=O)NCCN)CCC(=O)NCCN]

The terminal layer of the PAMAM dendrimer includes primary amine groups that can be modified to include functional groups or moieties known in the art or described herein.

The nanoparticle compositions described herein may be formed with G₀ to G₇ PAMAM dendrimers. PAMAM dendrimers are commercially available.

The homogeneous dendrimer may be polypropylenimine (PPI) dendrimer. PPI dendrimers may be G₀ to G₁₀ generation dendrimers. PPI dendrimers may be G₀ to G₁₀ generation dendrimers. PPI dendrimer may be but is not limited to, G₀ PPI dendrimers, G₁ PPI dendrimers, G₂ PPI dendrimers, G₃ PPI dendrimers, G₄ PPI dendrimers, G₅ PPI dendrimers, G₆ PPI dendrimers, G₇ PPI, G₈ PPI dendrimers, G₉ PPI dendrimers, or G₁₀ PPI dendrimers. For improved biocompatibility and low cytotoxicity, PPI dendrimers may be G₀ to G₂ generation dendrimers.

An exemplary molecular structure of G₁ diaminobutane amine polypropylenimine tetramine (DAB Am 4) of formula (III) is shown below.

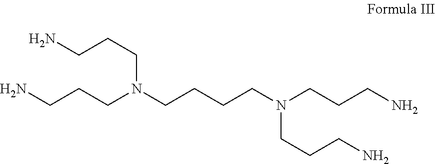 [SMILES: NCCCN(CCCN)CCCCN(CCCN)CCCN]

Diaminobutane amine polypropylenimine tetramine (DAB Am 4) of Formula III is a dendrimer having a 1,4-diaminobutane core (4-carbon core) and a terminal layer including 4 primary amino groups.

The homogeneous dendrimer may be polyethylenimine (PEI) dendrimer. The PEI dendrimer is also referred to as polyaziridine. The PEI dendrimer is a polymer comprising repeating units composed of an amine group and a two carbon aliphatic (CH₂—CH₂) spacer. PEI dendrimers may be G₀ to G₁₀ generation dendrimers. PEI dendrimer may be but is not limited to, G₀ PEI dendrimers, G₁ PEI dendrimers, G₂ PEI dendrimers, G₃ PEI dendrimers, G₄ PEI dendrimers, G₅ PEI dendrimers, G₆ PEI dendrimers, G₇ PEI, G₈ PPI dendrimers, G₉ PPI dendrimers, or G₁₀ PPI dendrimers. For improved biocompatibility and low cytotoxicity, PEI dendrimers may be G₀ to G₂ generation dendrimers. An exemplary molecular structure of a polyethylene imine monomer and repeating units a poly(ethylene-imine) (PEI) monomer of formula (VI) is shown below.

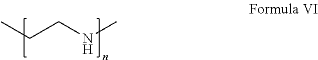

An exemplary scheme for modification of the terminal layer of homogeneous dendrimers described herein with fatty acids is provided below.

Scheme I depicts synthesis of an exemplary dendrimer by adding fatty acids to the terminal layer via amide coupling.

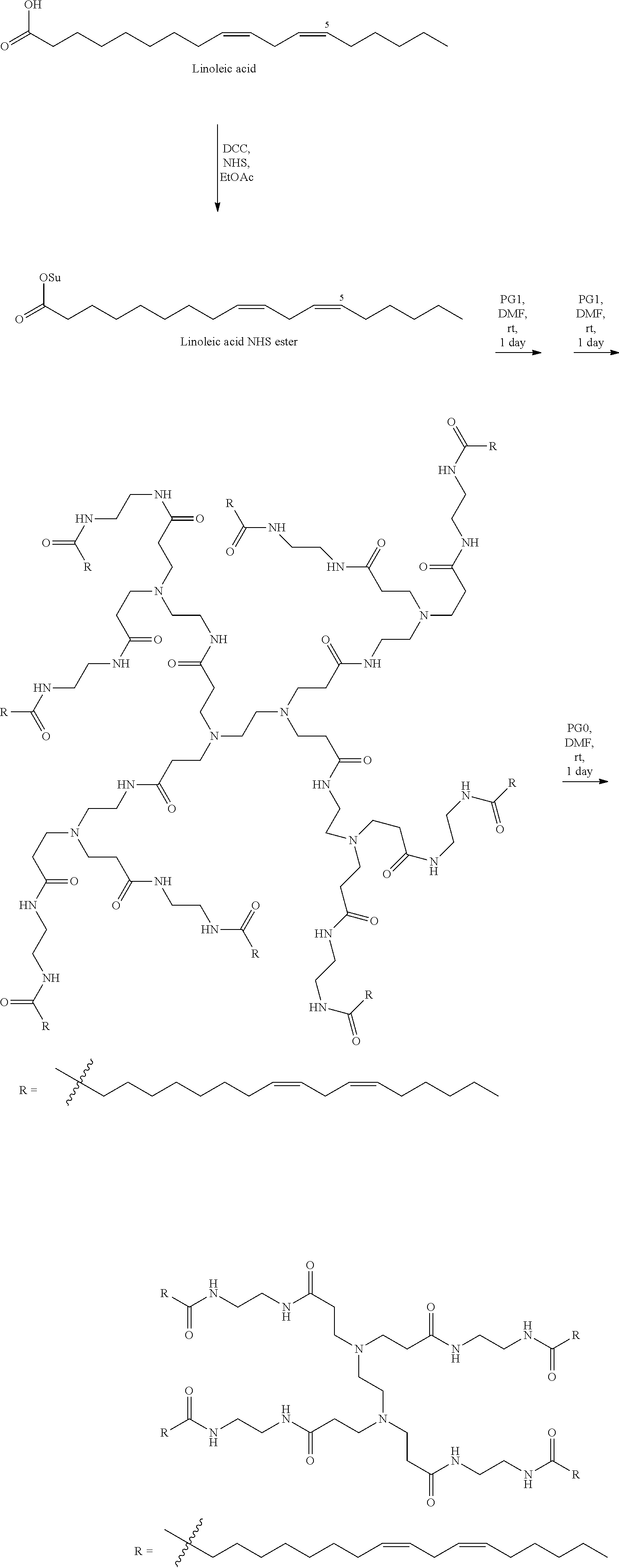

In the depicted process, linoleic acid and N-hydroxysuccinimide (NHS) dissolved in the ethyl acetate (EtOAc) are mixed with dicyclohexylcarbodiimide (DCC) to yield a linoleic acid NHS ester. The linoleic acid NHS ester dissolved in dimethylformamide (DMF) is added to G₁ PAMAM dendrimer (PG1; top) or G₀ PAMAM dendrimer (PG0; bottom). The reaction generally requires at least 24 hours and proceeds at room temperature. The reaction conditions are described in Example 1 herein.

In an embodiment, the heterogeneous modified dendrimer is provided. The heterogeneous modified dendrimer may contain moieties covalently bonded to the free amines to assist with self-assembly. The moieties that may assist self-assembly include, but are not limited to, alkanes, alkenes, alkynes, linear fluorinated carbons or fatty acids or derivatives thereof.

The ability to stay and act at the site of administration may be beneficial for delivery efficiency, reproducibility and performance because it prevents wasted payloads, unexpected tropism and off-target delivery, and off-target effects. These limitations may be solved by incorporating more amine groups per delivery molecules.

In an embodiment, additional amines may be added to one or more layers of the heterogeneous modified dendrimer. Additional amines may be added to a layer without adding more layers. The high amine density may be used to prevent nanoparticle recycling after initial endocytosis as it may amplify the proton-pump effect to more quickly rupture the endosomes post-uptake of the nanoparticles comprising the modified dendrimer.

To further prevent nanoparticle recycling, a modified dendrimer may have its terminal layer substituted with unsaturated alkyl groups, which are more fluid (lower crystallization temperature), and thus, capable of morphologically changing into a fusogenic form to help rupture the endosome. Thus, the nanoparticles including such modified dendrimers may become restricted and remain inside the cells at the site of injection only and may not be trafficked elsewhere.

In an embodiment, a nanoparticle composition comprising any one of the modified dendrimers described herein is provided.

The nanoparticle composition may comprise a mixture of modified dendrimers with distinct amounts or levels of tertiary or secondary amine substitution. Heterogeneous modified dendrimers containing only terminal tertiary amines may possess self-assembly properties. Modified dendrimers containing only terminal secondary amines may have less steric hindrance, and allow for more nucleic acid to associate with the amine residue. Modified dendrimers with a mix of both secondary and tertiary terminal amines may act as a bridge for the two types of molecules. Thus, the ratio used to blend the different types of modified dendrimers may allow one to further control the amount of nucleic acid payload that can be integrated, the degree/speed of self-assembly, the free energy of the nanoparticle, and how tightly bound the nucleic acid payload will be.

In an embodiment, a nanoparticle composition may comprise a mixture of two dendrimers each one of them comprising different levels of substitutions. These dendrimers may be mixed at a fixed ratio. A ratio may be 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, or 2:1 (w/w) or any ratio in a range between any two of the foregoing (endpoints inclusive).

In an embodiment, a nanoparticle composition may comprise a mixture of three dendrimers each one of them comprising different levels of substitutions. These dendrimers may be mixed at a fixed ratio. A ratio of the first modified dendrimer to the second modified dendrimer and to the third modified dendrimer may equal to one of 1:1:1, 1:1:2, 1:1:3, 1:1:4, 1:1:5, 1:1:6, 1:1:7, 1:1:8, 1:1:9, 1:1:10, 1:1:15, 1:1:20, 1:2:1, 1:3:1, 1:4:1, 1:5:1, 1:6:1, 1:7:1, 1:8:1, 1:9:1, 1:10:1, 1:15:1, 1:20:1, 1:2:2, 1:3:2, 1:4:2, 1:5:2, 1:6:2, 1:7:2, 1:8:2, 1:9:2, 1:10:2, 1:15:2, 1:20:2, 1:2:3, 1:3:3, 1:4:3, 1:5:3, 1:6:3, 1:7:3, 1:8:3, 1:9:3, 1:10:3, 1:15:3, 1:20:3, 1:2:4, 1:3:4, 1:4:4, 1:5:4, 1:6:4, 1:7:4, 1:8:4, 1:9:4, 1:10:4, 1:15:4, 1:20:4, 1:2:5, 1:3:5, 1:4:5, 1:5:5, 1:6:5, 1:7:5, 1:8:5, 1:9:5, 1:10:5, 1:15:5, 1:20:5, 1:2:6, 1:3:6, 1:4:6, 1:5:6, 1:6:6, 1:7:6, 1:8:6, 1:9:6, 1:10:6, 1:15:6, 1:20:6, 1:2:7, 1:3:7, 1:4:7, 1:5:7, 1:6:7, 1:7:7, 1:8:7, 1:9:7, 1:10:7, 1:15:7, 1:20:7, 1:2:8, 1:3:8, 1:4:8, 1:5:8, 1:6:8, 1:7:8, 1:8:8, 1:9:8, 1:10:8, 1:15:8, 1:20:8, 1:2:9, 1:3:9, 1:4:9, 1:5:9, 1:6:9, 1:7:9, 1:8:9, 1:9:9, 1:10:9, 1:15:9, 1:20:9, 1:2:10, 1:3:10, 1:4:10, 1:5:10, 1:6:10, 1:7:10, 1:8:10, 1:9:10, 1:10:10, 1:15:10, 1:20:10, 1:2:15, 1:3:15, 1:4:15, 1:5:15, 1:6:15, 1:7:15, 1:8:15, 1:9:15, 1:10:15, 1:15:15, 1:20:15, 1:2:20, 1:3:20, 1:4:20, 1:5:20, 1:6:20, 1:7:20, 1:8:20, 1:9:20, 1:10:20, 1:15:20, 1:20:20, 2:1:1, 3:1:1, 4:1:1, 5:1:1, 6:1:1, 7:1:1, 8:1:1, 9:1:1, 10:1:1, 15:1:1, or 20:1:1 (w/w/w) or any ratio in a range between any two of the foregoing (endpoints inclusive).

In an embodiment, a nanoparticle composition may comprise a mixture of the modified dendrimers consisting of four, five, six or more degrees of substitution. The modified dendrimers with different degrees of substitutions may be mixed with each other at a fixed ratio.

In an embodiment, a mixture may comprise modified dendrimers that are positional isomers. The positional isomers herein are structurally similar modified dendrimers that differ from one another by the location of the functional group or groups in the terminal layer.

In an embodiment, a nanoparticle composition may be a defined composition. As used herein, the term “defined composition” refers to a nanop article composition comprising a mixture of modified dendrimers, each one of them containing a discrete degree of substitution.

In an embodiment, the nanoparticle composition may also comprise one or more therapeutic or immunogenic nucleic acid agents. As used herein, the term “nucleic acid” refers to any natural or synthetic DNA or RNA molecules.

In an embodiment, the therapeutic or immunogenic nucleic acid agent may be an RNA or DNA molecule. The term “DNA” or “DNA molecule” or “deoxyribonucleic acid molecule” refers to a polymer of deoxyribonucleotides. The DNA molecule may be a polynucleotide, oligonucleotide, DNA, or cDNA. The DNA molecule may encode wild-type or engineered proteins, peptides or polypeptides, such as antigens. The term “RNA” or “RNA molecule” or “ribonucleic acid molecule” refers to a polymer of ribonucleotides (e.g., 2, 3, 4, 5, 10, 15, 20, 25, 30, or more ribonucleotides). The RNA molecule may be a replicon RNA (repRNA), small interfering RNA (siRNA), miRNA, single strand guide RNA (sgRNA), messenger RNA (mRNA), or transfer RNA (tRNA). The replicon RNA (repRNA) refers to a replication-competent, progeny-defective RNA virus genome that is incapable of producing infectious progeny virions. Viral genomes that are typically modified for use as repRNAs include “positive strand” RNA viruses. The modified viral genomes function as both mRNA and templates for replication. The small interfering RNA (siRNA) refers to an RNA (or RNA analog) comprising between about 10-50 nucleotides (or nucleotide analogs) which is capable of directing or mediating RNA interference. The microRNAs (miRNAs) refers to small (20-24 nt) regulatory non-coding RNAs that are involved in post-transcriptional regulation of gene expression in eukaryotes by affecting either or both the stability and translation of coding mRNAs. The messenger RNAs (mRNAs) are single-stranded RNAs that define the amino acid sequence of one or more polypeptide chains This information is translated during protein synthesis when ribosomes bind to the mRNA. The DNA or RNA molecules may be chemically modified.

The RNA molecule may be a monocistronic or polycistronic mRNA. A monocistronic mRNA refers to an mRNA comprising only one sequence encoding a protein, polypeptide or peptide. A polycistronic mRNA typically refers to two or more sequences encoding two or more proteins, polypeptides or peptides. An mRNA may encode a protein, polypeptide, or peptide that acts as an antigen.

In an embodiment, the DNA molecule may be a polynucleotide, oligonucleotide, DNA, or cDNA. The RNA molecule may be a replicon RNA (repRNA), small interfering RNA (siRNA), miRNA, single strand guide RNA (sgRNA), messenger RNA (mRNA), or transfer RNA (tRNA). The therapeutic or immunogenic nucleic acid agent may be non-covalently bound or covalently bound to the drug delivery molecule. The therapeutic or immunogenic nucleic agent may be electrostatically bound to the charged drug delivery molecule through an ionic bond.

In an embodiment, the nanop article compositions described herein may include immunogenic or therapeutic nucleic acid agents encoding antigens.

“Antigen” as used herein is defined as a molecule that triggers an immune response. The immune response may involve either antibody production, or the activation of specific immunologically active cells, or both. The antigen may refer to any molecule capable of stimulating an immune response, including macromolecules such as proteins, peptides, or polypeptides. The antigen may be a structural component of a pathogen, or a cancer cell. The antigen may be synthesized, produced recombinantly in a host, or may be derived from a biological sample, including but not limited to a tissue sample, cell, or a biological fluid.

The antigen may be but is not limited to a vaccine antigen, parasite antigen, bacterial antigen, tumor antigen, environmental antigen, therapeutic antigen or an allergen. As used herein a nucleotide vaccine is a DNA- or RNA-based prophylactic or therapeutic composition capable of stimulating an adaptive immune response in the body of a subject by delivering antigen(s). The immune response induced by vaccination typically results in development of immunological memory, and the ability of the organism to quickly respond to subsequent encounter with the antigen or infectious agent.

In an embodiment, the nanoparticle composition may be formulated to include drugs that contain negative or partially negative charges. The negatively charged drugs may be ionic drugs. The term “ionic drug” refers to an electrically asymmetric molecule, which is water soluble and ionizable in solution of distilled water. The ionic drugs may contain phosphate, phosphonate, or phosphinate functional groups. The drugs including phosphate groups may be phosphate-containing nucleotide analogs, for example, drugs used for treating cancer and viral chemotherapy. The phosphate-containing drugs may be but are not limited to purine and pyrimidine nucleoside analogs, Arabinosylcytosine (ara-C), Ara-C monophosphate (ara-CMP), azidothymidine (AZT), AZT monophosphate (AZTMP), 2′3′-dideoxycytidine (ddCD), cyclic adenoside monophosphate (cAMP), tenofovir, or adefovir.

The partially charged drugs may contain sulfone functional groups. The drugs including sulfone functional groups may be sulfonylurea drugs, for example, acetohexamide, chlorpropamide, tolbutamide, glibenclamide, glipizide, glimepiride, or gliclazide.

The nanoparticles may be formulated via the electrostatic association of the negative charge with the positive charge of the protonated amine groups in the modified dendrimer.

In an embodiment, the nanoparticle composition may comprise one or more small molecules. The small molecules may be zwitterionic molecules. The term “zwitterionic molecule” refers to a molecule with functional groups, of which at least one has a positive and one has a negative electrical charge and the net charge of the entire molecule is zero. The zwitterionic molecule may be an amino acid containing a basic amine group and acidic carboxylic group. The zwitterionic molecule may be a zwitterionic lipid, such as 1,2-distearoyl-sn-glycero-3-phosphocholine (DSPC).

In an embodiment, the nanoparticle composition may comprise one or more proteins, in particular, short-lived proteins.

In an embodiment, the nanoparticle composition may comprise one or more STING activators for induction or enhancement of an immune response. As used herein, “induction or enhancement of an immune response” refers to a statistically measurable induction or increase in an immune response caused by administration of a modified dendrimer comprising a STING activator compared to a control sample to which the modified dendrimer lacking the STING activator was administered. The enhanced immune response may be activated through “stimulator of interferon genes”-dependent signaling pathway. The induction or enhancement of the immune response may result in a prophylactic or therapeutic response in a subject. The enhanced immune response may result in an increased production of the Type I interferon (IFN), resistance to viral and/or bacterial infection, prevention or elimination of existing tumors.

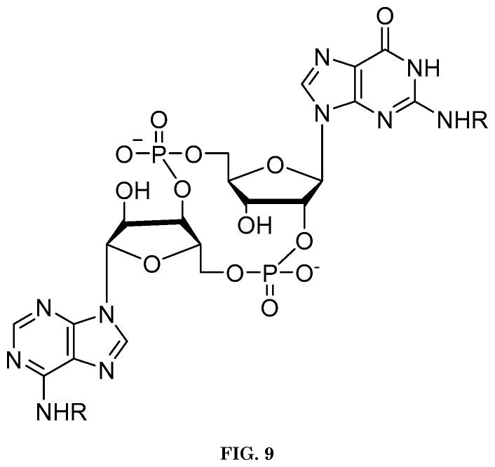

The term “STING activator” refers to nucleic acids or other molecules that bind a transmembrane STING protein causing stimulation of a STING-dependent Type I interferon response. The STING activator may be a cyclic purine including, but not limited to, adenine, guanine, inosine, hypoxanthine, xanthine, isoguanine, or other purines. Cyclic purines may be cyclic purine dinucleotides (CDNs), or derivatives thereof as described in WO2007/054279, published May 18, 2007; Yan et al., 2008, Bioorg Med Chem Lett. 18(20):5631; U.S. Pat. No. 7,592,326, issued Sep. 22, 2009; U.S. Pat. No. 7,709,458, issued May 4, 2010; Gao et al., 2013, Cell; 154(4):748-62; and U.S. patent application publication No. 20170333552, published Nov. 23, 2017, all of which are incorporated by reference herein as if fully set forth. FIG. 9 illustrates a non-limiting example of a cyclic dinucleotide, a cyclic guanosine monophosphate-adenosine monophosphate (2′3′-cGAMP), where one or both hydrogens (H) in the primary amines (NH₂) are substituted with hydrophobic R groups.

The hydrophobic R groups of the STING activator may react with the hydrophobic groups of modified dendrimer molecules and may assist with nanoparticle self-assembly by anchoring the STING activator to hydrophobic moieties found in the self-assembly-promoting layer a modified dendrimer. For example, the hydrophobic groups of the STING activator may associate with the hydrophobic alkyl groups of the fractal modified dendrimer. If the STING activator comprises CDNs, the anionic phosphate backbone of the CDN may be involved in electrostatic interaction with the cationic amine of the homogeneous or heterogeneous multilayer modified dendrimer. Thus, upon mixing with a nucleic acid agent, the STING activator may become a component of the nanoparticle. The inclusion of the STING activator may improve the Type I IFN response to a given antigen. In the case of RNA vaccines, the presence of the STING activator may require coordinating the timing of exposure to STING and the antigen to ensure that the resultant IFN response does not jeopardize the expression potency of the nucleic acid payload.

In an embodiment, the nanoparticle composition may comprise an adjuvant. The term “adjuvant” refers to a pharmacological or immunological agent or composition that modifies the effect of other agents, for example, drugs or vaccines. The adjuvant may be a molecule, or a substance that enhances accelerates, or prolongs an antigen-specific immune response when applied in combination with vaccine antigens. The adjuvant may be a DNA or RNA construct encoding a STING protein. A STING protein may be a wild type STING protein. The wild type STING protein may comprise an amino acid sequence with at least 70, 72, 75, 80, 85, 90, 91, 92, 93, 94, 95, 96, 97, 98, 99, or 100% identity to a reference sequence of SEQ ID NO: 1. A STING protein may be a mutant STING protein. The mutant of the STING protein may but is not limited to N154S, R284M, or N154S/R284M mutant protein. The N154S mutant protein may comprise an amino acid sequence with at least 70, 72, 75, 80, 85, 90, 91, 92, 93, 94, 95, 96, 97, 98, 99, or 100% identity to a reference sequence of SEQ ID NO: 3. The R284M mutant protein may comprise an amino acid sequence with at least 70, 72, 75, 80, 85, 90, 91, 92, 93, 94, 95, 96, 97, 98, 99, or 100% identity to a reference sequence of SEQ ID NO: 5. The N154S/R284M mutant protein may comprise an amino acid sequence with at least 70, 72, 75, 80, 85, 90, 91, 92, 93, 94, 95, 96, 97, 98, 99, or 100% identity to a reference sequence of SEQ ID NO: 7.

The DNA construct encoding the wild type STING protein may comprise a nucleic acid sequence with at least 70, 72, 75, 80, 85, 90, 91, 92, 93, 94, 95, 96, 97, 98, 99, or 100% identity to a reference sequence of SEQ ID NO: 2. The DNA construct encoding the N154S mutant protein may comprise a nucleic acid sequence with at least 70, 72, 75, 80, 85, 90, 91, 92, 93, 94, 95, 96, 97, 98, 99, or 100% identity to a reference sequence of SEQ ID NO: 4. The DNA construct encoding the R284M mutant protein may comprise a nucleic acid sequence with at least 70, 72, 75, 80, 85, 90, 91, 92, 93, 94, 95, 96, 97, 98, 99, or 100% identity to a reference sequence of SEQ ID NO: 6. The DNA construct encoding the N154S/R284M mutant protein may comprise a nucleic acid sequence with at least 70, 72, 75, 80, 85, 90, 91, 92, 93, 94, 95, 96, 97, 98, 99, or 100% identity to a reference sequence of SEQ ID NO: 8.

The RNA construct encoding the wild type STING protein may comprise a nucleic acid sequence with at least 70, 72, 75, 80, 85, 90, 91, 92, 93, 94, 95, 96, 97, 98, 99, or 100% identity to a reference sequence of SEQ ID NO: 9. The RNA construct encoding the N154S mutant protein may comprise a nucleic acid sequence with at least 70, 72, 75, 80, 85, 90, 91, 92, 93, 94, 95, 96, 97, 98, 99, or 100% identity to a reference sequence of SEQ ID NO: 10. The RNA construct encoding the R284M mutant protein may comprise a nucleic acid sequence with at least 70, 72, 75, 80, 85, 90, 91, 92, 93, 94, 95, 96, 97, 98, 99, or 100% identity to a reference sequence of SEQ ID NO: 11. The RNA construct encoding the N154S/R284M mutant protein may comprise a nucleic acid sequence with at least 70, 72, 75, 80, 85, 90, 91, 92, 93, 94, 95, 96, 97, 98, 99, or 100% identity to a reference sequence of SEQ ID NO: 12.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity is measured by the Smith Waterman algorithm (Smith TF, Waterman MS 1981 “Identification of Common Molecular Subsequences,” J Mol Biol 147: 195-197, which is incorporated herein by reference as if fully set forth).

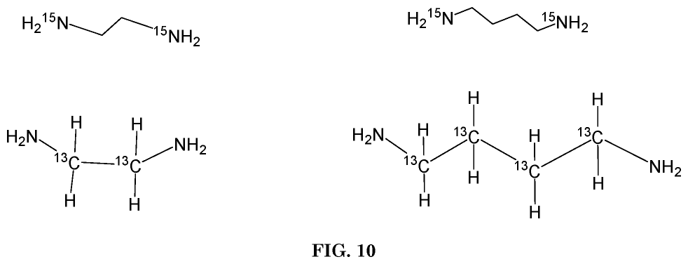

In an embodiment, the nanoparticle composition may comprise modified dendrimers containing functional groups suitable for tracking. To facilitate tracking of the delivery material in vitro and in vivo, homogeneous or heterogeneous modified dendrimer may have their cores contain stable isotopes of carbon (C) or nitrogen (N), such as ¹³C or ¹⁵N. FIG. 10 illustrates structures of one-layer of modified dendrimers with the cores containing stable isotopes of nitrogen (¹⁵N; top structures) and carbon (¹³C; bottom structures). When the modified dendrimers are formulated into nanoparticles with nucleic acids, such as replicon mRNA, they can be tracked in vitro and in vivo post-administration by techniques such as mass spectroscopy or nuclear magnetic resonance imaging. The inclusion of the stable isotopes makes identification of the delivery molecules easier as it is different from the abundant ¹²C and ¹⁴N isotopes that are dominantly found in tissues. Tracking may be useful for identifying biodistribution, material clearance and molecular stability of nanoparticles post-administration, and related issues.

In an embodiment, the nanoparticle composition may include amphiphilic polymers. The amphiphilic polymers may include hydrophobic and hydrophilic components.

In an embodiment, the hydrophobic component may be a phospholipid. The phospholipids may be but is not limited to ceramides, phosphatidylethanolamines, lysolipids, cholesterol, lysophospholipids or sphingolipids. Ceramides may be short chain (C₁-C₈), intermediate chain (C₉-C₁₄) or long chain (C₁₅-C₂₀) fatty amides, or fatty acid derivatives. The phosphatidylethanolamine may be a saturated or unsaturated phosphatidyl-ethanolamine. The hydrophobic component may be a neutral, cationic or anionic lipid. The neutral, cationic and anionic lipids may include, but are not limited to 1,2-diacyl-glycero-3-phosphocholines; phosphatidylserine, phosphatidylglycerol, phosphatidylinositol; glycolipids; phosphatidylcholine, sphingophospholipids, sphingomyelin, sphingo-glycolipids, ceramide galactopyranoside, gangliosides and cerebrosides; fatty acids, sterols containing a carboxylic acid group, i.e., cholesterol or derivatives thereof; and 1,2-diacyl-sn-glycero-3-phosphoethanolamines, including 1,2-dioleoyl-sn-Glycero-3-phosphoethanolamine 1,2-dioleolylglyceryl phosphatidylethanolamine, 1,2-dihexadecylphosphoethanolamine, 1,2-distearoylphosphatidylcholine, 1,2-di-palmitoylphosphatidylcholine, or 1,2-dimyristoylphosphatidylcholine. The hydrophobic component may include trimethyl ammonium salts (TAP lipids), e.g., a methylsulfate salt. TAP lipids may include without limitation DOTAP (dioleoyl-), DMTAP (dimyristoyl-), DPTAP (dipalmitoyl-), and DSTAP (distearoyl-). The hydrophobic component may be a cationic lipid, e.g., dimethyldioctadecyl ammonium bromide (DDAB), 1,2-diacyloxy-3-trimethylammonium prop anes, or N-[1-(2,3-dioloyloxy)propyl]-N,N-dimethyl amine (DODAP). The hydrophobic component may include long chain (C₄-C₃₀) saturated alkane molecules.

In an embodiment, the hydrophilic component may be a hydrophilic polymer. The hydrophilic polymer may include poly 3-amino esters and 1, 2-amino alcohol lipids. The hydrophilic polymers may be alkyl-modified polymers, e.g., alkyl modified poly(ethylene glycol). The hydrophilic polymers may include poly(alkylene glycol), polysaccharides, poly(vinyl alcohol)s, polypyrrolidones, polyoxyethylene block copolymers or polyethylene glycol (PEG). The hydrophilic polymers may be polyethylene glycol (PEG). PEG is one of the most commonly used protecting agents.

In an embodiment, the amphiphilic polymer may be a PEG-lipid conjugate.

The size, relative quantity and distribution of the amphiphilic polymer, such as the PEG-lipid polymer, included in the nanoparticle composition may affect physical properties of the nanoparticle composition, i.e., the efficacy of the intra-cellular delivery of therapeutic and immunogenic nucleic acid agents, and/or the efficacy of uptake of the nanoparticles by cells.

In an embodiment, a method of improving colloidal stability and self- assembly of a nanop article composition is provided. The method may comprise mixing a first, second and third modified dendrimers described herein to form a mixture. The first modified dendrimer may comprise a low level of substitutions of amine groups in a terminal layer. The low level of substitutions may be from 50% to 74% of substitutions of amine groups in the terminal layer. The low level may be 50%, 55%, 60%, 65%, 70% or 74% substitutions of amine groups in the terminal layer, or any value in a range between any two of the foregoing (endpoints inclusive). The low level may be less than 75% substitutions of amine group in the terminal layer. The second modified dendrimer may comprise an intermediate level of substitutions of amine group in the terminal layer. The intermediate level of substitutions may be from 75% to 99% of substitutions of amine groups in the terminal layer. The intermediate level may be 75%, 80%, 85%, 90%, 95% or 99% substitutions of amine groups in the terminal layer, or any value in a range between any two of the foregoing (endpoints inclusive). The intermediate level may be greater than 75% and less than 100% substitutions of amine groups in the terminal layer. The third modified dendrimer may comprise a high level of substitutions of amine groups in the terminal layer. The high level of substitutions may be 100% of substitutions of amine groups in the terminal layer.

The method may further comprise combining a mixture with a therapeutic or immunogenic agent to form a nanoparticle composition. The therapeutic or immunogenic agent may be any one of the nucleic acids described herein.

The first modified dendrimer with low level of substitutions, or low substitution dendrimer, may reduce steric hindrance of the nanoparticle compositions allowing more nucleic acid payload to electrostatically attach to the delivery molecule. The third modified dendrimer with high levels of substitutions, or high substitution dendrimer, may promote greater steric hindrance of the nanoparticle composition. Thus, the nanoparticle composition may not electrostatically attach as much nucleic acid payload as the modified dendrimers with low levels of substitutions. However, high substitution dendrimer may promote greater degree of self-assembly of the nanoparticles due to the substituted tails. The second modified dendrimer with intermediate levels of substitutions, or intermediate substituted dendrimer, may not only electrostatically attach nucleic acid payload, but may also act as a bridge between the high and low substituted dendrimers of the delivery molecule, thus allowing components to combine into a single nanoparticle.

In an embodiment, a method of manufacturing a defined nanoparticle composition is provided. The method may comprise mixing a plurality of modified dendrimers containing discrete degrees of substitution. The plurality of the modified dendrimers may be mixed with each other at a fixed ratio

In an embodiment, a method of manufacturing a nanop article composition capable of changing the rate of nucleic acid release in a cytoplasm of the cell is provided. The method comprises formulating the nanop article composition at different pH values. For slow release, the nanoparticles may be formulated at a low pH value. The low pH value may be any value in the range from pH 3.0 to pH 3.4 (endpoints inclusive). The low pH value may be 3.0, 3.1, 3.2, 3.3, or 3.4, or any value in between any two integers described herein. The low pH value may be less than 3.5. The amine groups of the modified dendrimers may become protonated, increasing their charge density, and may form more electrostatic associations with the nucleic acid payloads of the nanoparticle composition. The resulted binding of the nucleic acids to the carrier, may slow down the ultimate release of the nucleic acid into the cytoplasm of the cell. For an intermediate rate of release, the nanop articles may be formulated at a pH value in the range from 3.5 to 4.4 (endpoints inclusive). The intermediate pH value may be 3.5, 3.6, 3.7, 3.8, 3.9, 4.0, 4.1, 4.2, 4.3, or 4.4, or any value in between any two integers described herein. The intermediate pH value may be less than 4.5. For fast release, the nanoparticles may be formulated at a pH in the range from 4.5 to 6.5 (endpoints inclusive). The high pH value may be 4.5, 4.6, 4.7, 4.8, 4.9, 5.0, 5.1, 5.2, 5.3, 5.4, 5.5., 5.6, 5.7, 5.8, 5.9, 6.0, 6.1, 6.2, 6.3, 6.4, or 6.5, or any value in between any two integers described herein. With fewer H⁺ ions available, there may be less protonation of the amine groups and fewer electrostatic associations between the nucleic acid and the delivery material. This reduced electrostatic association may result in weak binding, and faster release of the nucleic acid payload inside the cell.

In an embodiment, a method of controlling physical properties a nanoparticle composition is provided. The method may comprise mixing any one of the nanoparticle compositions described herein and an amphiphilic polymer to form a mixture. The physical properties that can be controlled may be but are not limited to a diameter of the nanop article, the propensity of the nanop articles to aggregate, the number of nucleic acid molecules inside each nanop article, or the concentration of the nanop articles in the nanop article composition.

The mixture may contain 40% (w/w) or less of the amphiphilic polymer per nanoparticle composition. The mixture may comprise about 40% (w/w), about 35% (w/w), about 30% (w/w), about 25% (w/w), about 20% (w/w), about 15% (w/w), about 10% (w/w), about 5%(w/w), about 4% (w/w), about 3% (w/w), about 2% (w/w) or about 1% (w/w), or any amount in between any two integers described herein of the amphiphilic polymer per nanoparticle composition. The mixture comprising the amphiphilic polymer may comprise nanoparticles with a smaller diameter than nanoparticles of the composition lacking the amphiphilic polymer. The mixture may also comprise nanop articles having a higher propensity of the nanop articles to aggregate than nanoparticles of the composition lacking the amphiphilic polymer.

In an embodiment, a method for treating or preventing a disease or condition in a subject is provided. The method may comprise providing any one of the nanoparticle compositions described herein. The method may also comprise administering a therapeutically effective amount of the nanop article composition to a subject.

As used herein, the term “therapeutically effective amount” refers to the amount of nanop article composition which is effective for producing some desired therapeutic effect in at least a sub-population of cells in an animal at a reasonable benefit/risk ratio applicable to any medical treatment. A “therapeutically effective amount” refers to the amount sufficient to generate appearance of antigen-specific antibodies in serum, or disappearance of disease symptoms. Disappearance of disease symptoms may be assessed by decrease of virus in faeces or in bodily fluids or in other secreted products. The nanoparticle compositions may be administered using any amount and any route of administration effective for generating an immune response.

Therapeutic efficacy may depend on effective amounts of active agents and time of administering necessary to achieve the desired result. Administering a nanoparticle composition may be a preventive measure. Administering of a nanoparticle composition may be a therapeutic measure to promote immunity to the infectious agent, to minimize complications associated with the slow development of immunity especially in patients with a weak immune system, elderly or infants.

The exact dosage may be chosen by the physician based on a variety of factors and in view of individual patients. Dosage and administration may be adjusted to provide sufficient levels of the active agent or agents or to maintain the desired effect. For example, factors which may be taken into account include the type and severity of a disease; age and gender of the patient; drug combinations; and an individual response to therapy.

Therapeutic efficacy and toxicity of active agents in a nanoparticle composition may be determined by standard pharmaceutical procedures, for example, by determining the therapeutically effective dose in 50% of the population (ED50) and the lethal dose to 50% of the population (LD50) in cells cultured in vitro or experimental animals. Nanoparticle compositions may be evaluated based on the dose ratio of toxic to therapeutic effects (LD50/ED50), called the therapeutic index, the large value of which may be used for assessment. The data obtained from cell and animal studies may be used in formulating a dosage for human use.

The therapeutically effective dose may be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

The therapeutic dose shown in examples herein may be between 0.0001 μg and 1 mg of the therapeutic or immunogenic nucleic acid per kg body weight of the subject, or between 0.00001 μg and 1 mg (μg) units/dose/subject, and may be administered on a daily basis. However, doses greater than 1 mg may be provided. For example, the dose may be at least one milligram, or about 3×1 mg, or about 10×1 mg unit of nucleic acid/dose/subject. As nanop article vaccines may be readily produced and inexpensively engineered and designed and stored, greater doses for large animal subjects may be economically feasible. For an animal subject several orders of magnitudes larger that the experimental animals used in examples herein, the dose may be easily adjusted, for example, to about 3×10×1 μg, or about 3×20×1 μg, or about 3×30×1 μg for animals such as humans and small agricultural animals. However, doses of about 3×40×1 μg, 3×50×1 μg or even about 3×60×1 μg, for example, for a high value zoo animal or agricultural animal such as an elephant, may be provided. For preventive immunization, or periodic treatment, or treatment of a small wild animal, smaller doses such as less than about 3×1 μg, less than about 1 μg, less than about ½ 1 μg, less than about 250 ng, less than about 100 ng, less than about 50 ng, less than about 25 ng, less than about 10 ng, less than about 5 ng, less than about 1 ng, less than about ½ 1 ng, less than about 250 pg, less than about 100 pg, per dose may be provided. The therapeutic and immunogenic nucleic acid may be a combination of different nucleic acids used per treatment dose. The terms “subject” and “individual” are used interchangeably herein, and mean a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In an embodiment, the subject may be a mammal, e.g., a primate, e.g., a human. The terms, “patient” and “subject” are used interchangeably herein. The terms, “patient” and “subject” are used interchangeably herein.

Preferably, the subject is a mammal. The mammal may be a human, non-human primate, mouse, rat, dog, cat, horse, or cow, but are not limited to these examples. Mammals other than humans may be advantageously used as subjects that represent animal models of a disease or disorder. In addition, the methods described herein may be used to treat domesticated animals and/or pets. A subject may be male or female.

As used herein, the term “administer” refers to the placement of a composition into a subject by a method or route which results in at least partial localization of the composition at a desired site such that desired effect is produced. A nanoparticle composition described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration.

Exemplary modes of administration include, but are not limited to, injection, infusion, instillation, inhalation, or ingestion. “Injection” includes without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrastemal injection and infusion. In an embodiment, the compositions may be administered by intravenous infusion or injection.

The nanoparticle compositions may be used for delivery of therapeutic or immunogenic nucleic acids for gene targeting. The therapeutic or immunogenic nucleic acid may be an antisense oligonucleotide (AON) or a double-stranded small interfering RNA (siRNA). Typically, siRNAs are between 21 and 23 nucleotides in length. The siRNAs may comprise a sequence complementary to a sequence contained in an mRNA transcript of a target gene when expressed within the host cell. The antisense oligonucleotide may be a Morpholino antisense oligonucleotide. The antisense oligonucleotide may include a sequence complementary to a sequence contained in an mRNA transcript of a target gene. The therapeutic or immunogenic nucleic acid may be an interfering RNA (iRNA) against a specific target gene within a specific target organism. The iRNA may induce sequence-specific silencing of the expression or translation of the target polynucleotide, thereby down-regulating or preventing gene expression. The iRNA may completely inhibit expression of the target gene. The iRNA may reduce the level of expression of the target gene compared to that of an untreated control. The therapeutic or immunogenic nucleic acid may be a micro RNA (miRNA). The miRNA may be a short RNA, e.g., a hairpin RNA (hpRNA). The miRNA may be cleaved into biologically active dsRNA within the target cell by the activity of the endogenous cellular enzymes. The RNA may be a double stranded RNA (dsRNA). The ds RNA may be at least 25 nucleotides in length or may be longer. The dsRNA may contain a sequence that is complementary to the sequence of the target gene or genes.

In an embodiment, the therapeutic or immunogenic nucleic acid may be or may encode an agent that totally or partially reduces, inhibits, interferes with or modulates the activity or synthesis of one or more genes encoding target proteins. The target genes may be any genes included in the genome of a host organism. The sequence of the therapeutic or immunogenic nucleic acid may not be 100% complementary to the nucleic acid sequence of the target gene.

In an embodiment, the nanoparticle composition may be used for targeted, specific alteration of the genetic information in a subject. As used herein, the term “alteration” refers to any change in the genome in the cells of a subject. The alteration may be insertion or deletion of nucleotides in the sequence of a target gene. “Insertion” refers to addition of one or more nucleotides to a sequence of a target gene. The term “deletion” refers to a loss or removal of one or more nucleotides in the sequence of a target gene. The alteration may be correction of the sequence of a target gene. “Correction” refers to alteration of one or more nucleotides in the sequence of a target gene, e.g., by insertion, deletion or substitution, which may result in a more favorable expression of the gene manifested by improvements in genotype and/or phenotype of the host organism.

The alteration of the genetic information may be achieved via the genome editing techniques. As used herein, “genome editing” refers to the process of modifying the nucleotide sequence in the genome in a precise or controlled manner.

An exemplary genome editing system is a Clustered Regularly Interspaced Short Palindromic Repeats (CRISPR) system as described, for example, in WO 2018/154387, published Aug. 30, 2018, which is incorporated herein by reference as if fully set forth. In general, “CRISPR system” refers to transcripts and other elements involved in the expression of CRISPR-associated (Cas) genes, including sequences encoding a Cas gene, a tracr (trans-activating CRISPR) sequence, a tracr-mate sequence, a guide sequence, or other sequences and transcripts from a CRISPR locus. One or more tracr mate sequences may be operably linked to a guide sequence before processing or crRNA after processing by a nuclease. The tracrRNA and crRNA may be linked and may form a chimeric crRNA-tracrRNA hybrid where a mature crRNA is fused to a partial tracrRNA via a synthetic stem loop to mimic the natural crRNA:tracrRNA duplex as described in Cong et al., Science, 15:339(6121):819-823 (2013) and Jinek et al., Science, 337(6096):816-21 (2012), which are incorporated herein by reference as if fully set forth). A single fused crRNA-tracrRNA construct is also referred herein as a guide RNA or gRNA, or single-guide RNA (sgRNA). Within an sgRNA, the crRNA portion is identified as the “target sequence” and the tracrRNA is often referred to as the “scaffold.” In an embodiment, the nanoparticle compositions described herein may be used to deliver an sgRNA.

In an embodiment, the nanoparticle compositions may be used to apply other exemplary genome editing systems including meganucleases, homing endonucleases, TALEN-based systems, or Zinc Finger Nucleases. The nanoparticle compositions may be used to deliver the nucleic acid (RNA and/or DNA) that encodes the sequences for these gene editing tools, and the actual gene products, proteins, or other molecules.

In an embodiment, the nanoparticle composition may be used for gene targeting in a subject in vivo or ex vivo, e.g., by isolating cells from the subject, editing genes, and implanting the edited cells back into the subject.

The following list includes particular embodiments of the present invention. But the list is not limiting and does not exclude alternate embodiments, or embodiments otherwise described herein. Percent identity described in the following embodiments list refers to the identity of the recited sequence along the entire length of the reference sequence.

### Embodiments

1. A modified dendrimer comprises a plurality of terminal amine groups substituted with fatty acids or derivatives thereof.
2. The modified dendrimer of embodiment 1, wherein the dendrimer is selected from the group consisting of: a polyamidoamine (PAMAM) dendrimer, poly(propylene imine) (PPI) dendrimer and poly ethylene imine (PEI) dendrimer.
3. The modified dendrimer of one or both embodiments 1 and 2, wherein the modified dendrimer is a generation 0, generation 1, or generation 2 dendrimer.
4. The modified dendrimer of any one or more of embodiments 1-3, wherein the modified dendrimer comprises 100% of the terminal amine groups substituted with fatty acids or derivative thereof.
5. The modified dendrimer of any one or more of embodiments 1-4, wherein the fatty acids or the derivatives thereof are selected from the group consisting of: arachidonic acid, oleic acid, eicosapentanoic acid, lauric acid, caprylic acid, capric acid, myristic acid, palmitic acid, stearic acid, linoleic acid, and linolenic acid or esters thereof.
6. The modified dendrimer of any one or more of embodiments 1-5, wherein the modified dendrimer comprises a core selected from the group consisting of: ethylenediamine, diaminobutane, N¹-(2-aminoethyl) ethane, N¹-(2-aminoethyl)propane, N³-dimethylpropan-, N¹,N¹′-(ethane-1,2-diyl)bis(ethane), N¹-(2-(4-(2-aminoethyl)piperazin-1-yl)ethyl)ethane-1,2-diaminecyclohexan, N¹-(2-(4-(2-aminoethyl)piperazin-1-yl)ethyl)eth ane-1,2-diaminecyclohexan-, -poly(ethylene)-, -, N¹,N¹-bis(2-aminoethyl)ethane-1,2-diamine, trimesic acid/trimesoyl chloride, pentaerythritol, inositol, thiourea, hydrazinecarbothioamide, hydrazinecarbothiohydrazide, urea, 3-ureidopropanoic acid, ethane-1,2-diamine; ethane-1,2-diamine-¹⁵N₂; ethane-1,2-diamine-1,2-¹³C₂; butane-1,4-diamine; butane-1,2-diamine-¹⁵N₂; butane-1,4-diamine-1,2,3,4-¹³C₂; N¹-(2-aminoethyl)propane-1,3-diamine; N¹-(2-aminoethyl)-N¹-methylethane-1,2-diamine; N¹-methylpropane-1,3-diamine; N¹, N³-dimethylpropane-1,3-diamine; N¹-(2-aminoethyl)ethane-1,2-diamine; and N¹, N³-(ethane-1,2-dyl)bis(ethane-1,2-diamine) thiourea, hydrazinecarbothioamide, hydrazinecarbothiohydrazide, urea, 3-ureidopropanoic acid, 2,2′-(ethane-1,2-diylbis(oxy)bis(ethan-1-amine), 2,2′-(ethane-1,2-diylbis(azanediyl)bis(ethan-1-ol), 2-((2-aminoethyl)amino)ethan-1-ol; N¹, N¹-bis(2-aminoethyl)ethane-1,2-diamine; N¹-(2-(4-(2-aminoethyl)piperazin-1-yl)ethyl)ethane-1,2-diamine; cyclohexane-1,2-diamine; poly(ethylene)1,n diamine; polyethylenimine, linear and polyethylenimine, branched.
7. The modified dendrimer of any one or more of embodiments 1-6, wherein the modified dendrimer comprises at least 6 amine groups per molecule.
8. The modified dendrimer of any one or more of embodiments 1-7, wherein the modified dendrimer comprises a tracking moiety.
9. The modified dendrimer of embodiment 8, wherein the tracking moiety is a stable isotope.
10. The modified dendrimer of any one or both of embodiments 9-10, wherein the stable isotope is a stable isotope of carbon or nitrogen.
11. The modified dendrimer of embodiment 10, wherein the stable isotope of carbon is ¹³C.
12. The modified dendrimer of embodiment 10, wherein the stable isotope of nitrogen is ¹⁵N.
13. A nanoparticle composition comprising the modified dendrimer of any one or more of embodiments 1-12, and a therapeutic or immunogenic nucleic acid enclosed within the nanoparticle composition.
14. The nanoparticle composition of embodiment 13 further comprising an immune modulating agent.
15. The nanoparticle composition of any one or both of embodiments 13-14, wherein the immune modulating agent is a STING activator.
16. The nanoparticle composition of any one of more of embodiments 13-15, wherein the STING activator comprises a cyclic-dinucleotide.
17. The nanoparticle composition of any one or more of embodiments 15-16, wherein the STING activator comprises a hydrophobic moiety.
18. The nanoparticle composition of embodiment 17, wherein the hydrophobic moiety is selected from the group consisting of: alkane, alkene, alkyne and saturated or unsaturated fluorinated carbon.
19. The nanoparticle composition of any one or more of embodiments 13-18, wherein the therapeutic or immunogenic nucleic acid agent is selected from the group consisting of: a polynucleotide, oligonucleotide, DNA, cDNA, RNA, repRNA, siRNA, miRNA, sgRNA, and mRNA.
20. The nanoparticle composition of any one or more of embodiments 13-19, wherein the therapeutic or immunogenic nucleic acid agent encodes one or more antigens selected from the group consisting of infectious disease, pathogen, cancer, autoimmunity disease and allergenic disease.
21. The nanoparticle composition of any one or more of embodiments 13-20, wherein the therapeutic or immunogenic nucleic acid agent comprises an RNA or DNA capable of silencing, inhibiting or modifying the activity of a gene.
22. The nanoparticle composition of any one or more of embodiments 13-21, wherein the therapeutic or immunogenic nucleic acid agent comprises at least one polynucleotide encoding a STING protein.
23. The nanoparticle composition of embodiment 22, wherein the STING protein comprises an amino acid sequence with at least 90% identity to a sequence selected from the group consisting of SEQ ID NOS: 1, 3, 5 and 7.
24. The nanoparticle composition of embodiment 22, wherein the at least one polynucleotide comprises a DNA sequence with at least 90% identity to a sequence selected from the group consisting of SEQ ID NOS: 2, 4, 6 and 8.
25. The nanoparticle composition of embodiment 22, wherein the at least one polynucleotide comprises an RNA sequence with at least 90% identity to a sequence selected from the group consisting of SEQ ID NOS: 9-12.
26. The nanoparticle composition of any one or more of embodiments 13-25 further comprising an amphiphilic polymer.
27. The nanoparticle composition of embodiment 26, wherein the amphiphilic polymer comprises a hydrophilic component selected from the group consisting of: polyalkylene oxides, block copolymers, and polyethylene glycol molecules.
28. The nanoparticle composition of any one or both of embodiments 26-27, wherein the amphiphilic polymer comprises 1,2-dimyristoyl-sn-glycero-3-phosphoethanolamine-N-[methoxy(poly-ethylene glycol)-2000].
29. The nanoparticle composition of any one or more of embodiments 26-28, wherein the amphiphilic polymer comprises a hydrophobic component selected from the group consisting of: lipid and a phospholipid.
30. The nanoparticle composition of any one or more of embodiments 13-29, wherein the nanoparticle composition comprises the amphiphilic polymer in a range from 1% (w/w) to 40% (w/w) of the amphiphilic polymer per nanoparticle composition.
31. A modified dendrimer comprising a core, a plurality of intermediate layers, and a terminal layer, wherein the plurality of intermediate layers comprises at least one layer modified for endosomal escape or at least one layer modified for hydroxide scavenging, or both.
32. The modified dendrimer of embodiment 31, wherein the core is selected from the group consisting of: N¹-(2-aminoethyl)ethane, N¹-(2-aminoethyl)propane, N³-dimethylpropan-, N¹,N¹′-(ethane-1,2-diyl)bis(ethane), N¹-(2-(4-(2-aminoethyl)piperazin-1-yl)ethyl)ethane-1,2-diaminecyclohexan, N¹-(2-(4-(2-aminoethyl)piperazin-1-yl)ethyl)ethane-1,2-diaminecyclohexan-, -poly(ethylene)-, -, N¹,N¹-bis(2-aminoethyl)ethane-1,2-diamine, trimesic acid/trimesoyl chloride, pentaerythritol, inositol, thiourea, hydrazinecarbothioamide, hydrazinecarbothiohydrazide, urea, 3-ureidopropanoic acid, ethane-1,2-diamine; ethane-1,2-diamine-¹⁵N₂; ethane-1,2-diamine-1,2-¹³C₂; butane-1,4-diamine; butane-1,2-diamine-¹⁵N₂; butane-1,4-diamine-1,2,3,4-¹³C₂; N¹-(2-aminoethyl)propane-1,3-diamine; N¹-(2-aminoethyl)-N¹-methylethane-1,2-diamine; N¹-methylpropane-1,3-diamine; N¹, N³-dimethylpropane-1,3-diamine; N¹-(2-aminoethyl)ethane-1,2-diamine; and N¹, N³-(ethane-1,2-dyl)bis(ethane-1,2-diamine) thiourea, hydrazinecarbothioamide, hydrazinecarbothiohydrazide, urea, 3-ureidopropanoic acid, 2,2′-(ethane-1,2-diylbis(oxy)bis(ethan-1-amine), 2,2′-(ethane-1,2-diylbis(azanediyl)bis(ethan-1-ol), 2- ((2-aminoethyl)amino)ethan-1-ol; N¹, N¹-bis(2-aminoethyl)ethane-1,2-diamine; N¹-(2-(4-(2-aminoethyl)piperazin-1-yl)ethyl)ethane-1,2-diamine; cyclohexane-1,2 diamine; poly(ethylene)1,n diamine; polyethylenimine, linear and polyethylene imine, branched.
33. The modified dendrimer of any one or both of embodiments 31-32, wherein the at least one layer modified for endosomal escape comprises a polyfluorocarbon.
34. The modified dendrimer of embodiment 33, wherein the polyfluorocarbon comprises at least one moiety is selected from the group consisting of nonafluoropentyl, tridecafluoroheptyl and heptadecafluorononyl groups.
35. The modified dendrimer of any one or more of embodiments 31-34, wherein at least one layer of the plurality of the intermediate layers comprises the functional moiety selected the group consisting of: C₁-C₁₇ chains (saturated and unsaturated), fluorinated carbons, methyl, ethyl, propyl, butyl, phenyl, benzyl, alpha-methylbenzyl, tosyl, N-oxo-(4-fluorophenyl), 1-hydroxyethyl, carboxylic acid, carboxylic acid salt, amide, methyl ester, ethyl ester, and tertbutyl ester groups.
36. The modified dendrimer of any one or more of embodiments 31-35, wherein the at least one layer modified for hydroxide ion-scavenging comprises a functional group selected from a carboxylic acid group or a sulfonic acid group.
37. The modified dendrimer of any one or more of embodiments 31-36, wherein the terminal layer is reacted with a compound selected from the group consisting of: oxirane, 2-methyloxirane, 2-ethyloxirane, 2-propyloxirane, 2-butyloxirane, 2-pentyloxirane, 2-hexyloxirane, 2-octyloxirane, 2-decyloxirane, 2-dodecyloxirane, 2-tridecyloxirane, 2-tetradecyloxirane, 2-pentadecyloxirane, 2-octadecyloxirane, 2-(but-3-en-1-yl)oxirane, 2-(oct-3-en-yl)oxirane, 2-(2,2,3,3,4,4,5,5,5-nonafluoropentyl) oxirane, 2-(2,2,3,3,4,4,5,5,6,6,7,7,7-tridecafluoroheptyl)oxirane, and 2-(2,2,3,3, 4,4,5,5,6,6,7,7,8,8,9,9,9-heptadecafluorononyloxirane, and a derivative thereof.
38. The modified dendrimer of one or more embodiments 31-37, wherein the modified dendrimer comprises the terminal layer comprising at least one moiety selected from the group consisting of hydrogen, methyl, ethyl, propyl, butyl, pentyl, hexyl, octyl, decyl, dodecyl, tridecyl, tetradecyl, pentadecyl, hexadecyl, octadecyl, but-3-en-lyl, oct-7-en-1-yl, 12-tridecenyl, 14-pentadecynyl, 17-octadecenyl, oleyl, 2,2,3,3,4,4,5,5,5-nonafluoropentyl, linoleyl, 2,2,3,3,4,4,5,5,6,6,7,7,7-tride cafluoroheptyl, arachidoneyl, and 2,2,3,3,4,4,5,5,6,6,7,7,8,8,9,9,9-hepta decafluoro- nonyl.
39. The modified dendrimer of any one or more of embodiments 31-38, wherein the terminal layer comprises an unsaturated alkyl group.
40. The modified dendrimer of embodiment 39, wherein the unsaturated alkyl group is selected from the group consisting of: alkenyl, or alkynyl groups, branched-chain alkyl, alkenyl, or alkynyl groups, alkyl groups containing alkyl, alkenyl or alkynyl braches, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, and alkynyl groups.
41. The modified dendrimer of any one or more of embodiments 31-40, wherein the modified dendrimer comprises at least 6 amine groups per molecule.
42. The modified dendrimer of any one or more of embodiments 31-41, wherein the core or at least one layer of the plurality of intermediate layers further comprises a tracking moiety.
43. The modified dendrimer of embodiment 42, wherein the tracking moiety is a stable isotope.
44. The modified dendrimer of embodiment 43, wherein the stable isotope is a stable isotope of carbon or nitrogen.
45. The modified dendrimer of any one or both of embodiments 43-44, wherein the stable isotope of carbon is ¹³C.
46. The modified dendrimer of or both of embodiments 43-44, wherein the stable isotope of nitrogen is ¹⁵N.
47. A nanoparticle composition comprising a modified dendrimer of any one or more of embodiments 31-46 and a therapeutic or immunogenic nucleic acid agent enclosed within the nanoparticle composition.
48. The nanoparticle composition of embodiment 47, wherein the therapeutic or immunogenic nucleic acid agent is selected from the group consisting of: a polynucleotide, oligonucleotide, DNA, cDNA, RNA, repRNA, siRNA, miRNA, sgRNA, and mRNA.
49. The nanoparticle composition of any one or both of embodiments 47-486, wherein the therapeutic or immunogenic nucleic acid agent encodes one or more antigens selected from the group consisting of infectious disease, pathogen, cancer, autoimmunity disease and allergenic disease.
50. The nanoparticle composition of any one or more of embodiments 47-49, wherein the therapeutic or immunogenic nucleic acid agent comprises an RNA or DNA capable of silencing, inhibiting or modifying the activity of a gene.
51. The nanoparticle composition of any one or more of embodiments 47-50, wherein the therapeutic or immunogenic nucleic acid agent comprises at least one polynucleotide encoding a STING protein.
52. The nanoparticle composition of embodiment 51, wherein the STING protein comprises an amino acid sequence with at least 90% identity to a sequence selected from the group consisting of SEQ ID NOS: 1, 3, 5 and 7.
53. The nanoparticle composition of embodiment 51, wherein the at least one polynucleotide comprises a DNA sequence with at least 90% identity to a sequence selected from the group consisting of SEQ ID NOS: 2, 4, 6 and 8.
54. The nanoparticle composition of embodiment 51, wherein the at least one polynucleotide comprises an RNA sequence with at least 90% identity to a sequence selected from the group consisting of SEQ ID NOS: 9-12.
55. The nanoparticle composition of any one or more of embodiments 47-54 further comprising an immune modulating agent.
56. The nanoparticle composition of embodiment 55, wherein the immune modulating agent is a STING activator.
57. The nanoparticle composition of embodiment 56, wherein the STING activator comprises a cyclic-dinucleotide.
58. The nanoparticle composition of any one or both of embodiments 56-57, wherein the STING activator comprises a hydrophobic moiety.
59. The nanoparticle composition of embodiment 58, wherein the hydrophobic moiety is selected from the group consisting of: alkane, alkene, alkyne and saturated or unsaturated fluorinated carbon.
60. The nanoparticle composition of any one or more of embodiments 47-59, wherein the modified dendrimer is a mixture of a first modified dendrimer, a second modified dendrimer and a third modified dendrimer, wherein the first modified dendrimer comprises a low level of substitutions of amine groups in a terminal layer, the second modified dendrimer comprises an intermediate level of substitutions of amine group in the terminal layer and the third modified dendrimer comprises a high level of substitutions of amine groups in the terminal layer.
61. The nanoparticle composition of any one or more of embodiments 47-60 further comprising an amphiphilic polymer.
62. The nanoparticle composition of embodiment 61, wherein the amphiphilic polymer comprises a hydrophilic component selected from the group consisting of: polyalkylene oxides, block copolymers, and polyethylene glycol molecules.
63. The nanoparticle composition of any one or both of embodiments 61-62, wherein the amphiphilic polymer comprises 1,2-dimyristoyl-sn-glycero-3-phosphoethanolamine-N-[methoxy (poly-ethylene glycol)-2000].
64. The nanoparticle composition of any one or more of embodiments 61-62, wherein the amphiphilic polymer comprises a hydrophobic component selected from the group consisting of: lipid and a phospholipid.
65. The nanoparticle composition of any one or more of embodiments 47-64, wherein the nanoparticle composition comprises 40% (w/w) or less of the amphiphilic polymer per nanoparticle composition.
66. The nanoparticle composition of any one or more of embodiments 47-65, wherein the nanoparticle composition comprises 15% (w/w) or more of the amphiphilic polymer per nanoparticle composition.
67. A method of manufacturing a nanoparticle composition capable of altering the rate of the nucleic acid release in cytoplasm of the cell comprising formulating the nanoparticle composition at pH ranging from 3.0 to 6.5.
68. The method of embodiment 67, wherein the nanop article composition is formulated at a pH value in the range from 3.0 to less than 3.5 for slow release of the nucleic acid in the cytoplasm of the cell.
69. The method of embodiment 67, wherein the nanop article composition is formulated at pH value in a range from 3.5 to less than 4.5 for an intermediate rate of release of the nucleic acid in the cytoplasm of the cell.
70. The method of embodiment 67, wherein the nanop article composition is formulated at pH value in a range from 4.5 to 6.5 for the fast release of the nucleic acid in the cytoplasm of the cell.
71. A method for treating or preventing a disease or condition in a subject comprising:

providing a nanoparticle composition of any one or more of embodiments 13-30 and 47-66; and

administering a therapeutically effective amount of the nanoparticle composition to a subject.
72. The method of embodiment 71, wherein the therapeutically effective amount of the nanoparticle composition comprises the therapeutic or immunogenic nucleic acid agent in a range from 0.01 mg nucleic acid to 10 mg nucleic acid per kg body weight of the subject.
73. The method of any one or both of embodiments 71-72, wherein the subject is a mammal.
74. The method of embodiment 73, wherein the mammal is selected from the group consisting of: a chicken, a rodent, a canine, a primate, an equine, a high value agricultural animal, and a human.
75. A method of controlling physical properties a nanoparticle composition comprising mixing the nanoparticle composition of any one or more of embodiments 13-30 and 47-66 and an amphiphilic polymer to form a mixture.
76. The method of embodiment 75, wherein the physical properties are selected from the group consisting of: a diameter of the nanop article, the propensity of the nanoparticles to aggregate, the number of nucleic acid molecules inside each nanop article, and the concentration of the nanop articles in the nanop article composition.
77. The method of any one or both of embodiments 75-76, wherein the mixture contains 40% (w/w) or less of the amphiphilic polymer and comprises nanoparticles with a smaller diameter than nanoparticles of the composition lacking the amphiphilic polymer.
78. The method of any one or more of embodiments 75-76, wherein the mixture contains 40% (w/w) or less of the amphiphilic polymer and comprises nanop articles having a higher propensity of the nanop articles to aggregate than nanop articles of the composition lacking the amphiphilic polymer.
79. A nanoparticle composition comprising a modified dendrimer and a nucleic acid comprising at least one polynucleotide encoding a STING protein.
80. A method of generating an immune response in a subject comprising administering to the subject a nanoparticle composition of any one or more of embodiments 13-30, 47-66 and 79.
81. A method of using a nanoparticle composition of any one or more of embodiments 13-30, 47-66 and 79 for treating or preventing a disease or condition in a subject.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims

Further embodiments herein may be formed by supplementing an embodiment with one or more elements from any one or more other embodiments herein, and/or substituting one or more elements from one embodiment with one or more elements from one or more other embodiments

### EXAMPLES

The following non-limiting examples are provided to illustrate particular embodiments. The embodiments throughout may be supplemented with one or more details from one or more examples below, and/or one or more elements from an embodiment may be substituted with one or more details from one or more examples below.

### Example 1. Nanoparticle Compositions Containing Dendrimers Modified with Fatty Acids

The transfection efficiency of PAMAM is affected by the generation of the dendrimer. A low generation PAMAM has fewer surface primary amines and less rigid surface structure, while a high generation PAMAM has more surface primary amines that form a rigidly spherical surface exhibiting high density of charges. It was reported that a low generation, such as Gᵢ, PAMAM was not able to complex nucleic acid because of the low positive charge density whereas a high generation PAMAM was able to complex with nucleic acids (Jensen et al., Int J Pharm. 2011; 416:410-418; and Chen et al., Langmuir, 2000, 16:15-19, which are incorporated herein by reference as if fully set forth). However, high generation PAMAM dendrimers exhibit cytotoxic and hemolytic properties (Palmerson Mendez et al., Molecules 2017, 22, 1401, which is incorporated herein by reference as if fully set forth). Besides determining physicochemical properties, the characteristics of the surface groups of the dendrimers also determine their biological activity and biocompatibility. The ideal gene delivery vehicle should be biocompatible to prevent bioaccumulation and subsequent cytotoxicity. There is a need for low generation dendrimers that can complex with nucleic acids and translocate across cellular membranes while maintaining biocompatibility and avoiding cytotoxicity.

In the present application, the terminal layer of low generation PAMAM cores (PAMAM-NH₂) were substituted with endogenous/essential fatty acid side chains through amide bonds, rendering them susceptible to hydrolysis in plasma by amidases, and thus biocompatible. Such low generation fatty acid chain dendrimers can be noncovalently combined with nucleic acids to form nanop articles through their dynamic equilibrating nature. Incorporation of fatty acid chains into lower generation dendrimers results in a carrier exhibiting the desired biocompatibility properties, capable of binding and transporting nucleic acids into cells.

It was observed that incorporation of the fatty acids in a terminal layer of G₀-G₂ non-toxic dendrimers resulted in self-assembly properties of nanoparticle compositions that include nucleic acids.

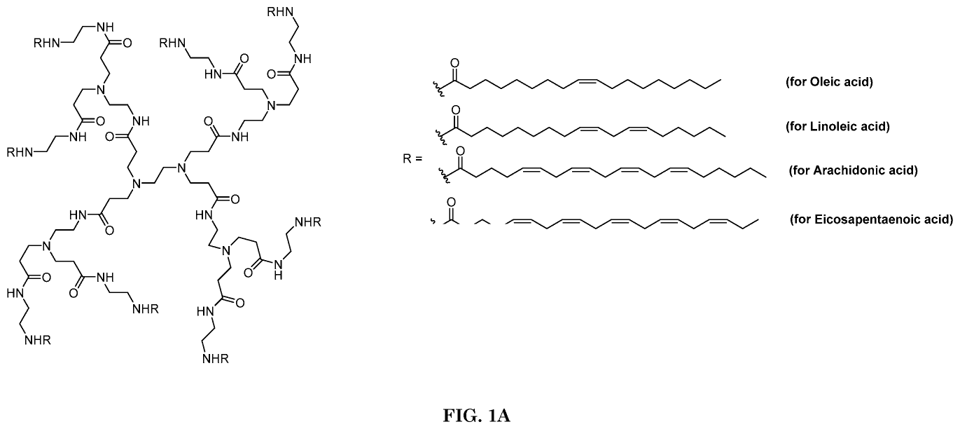

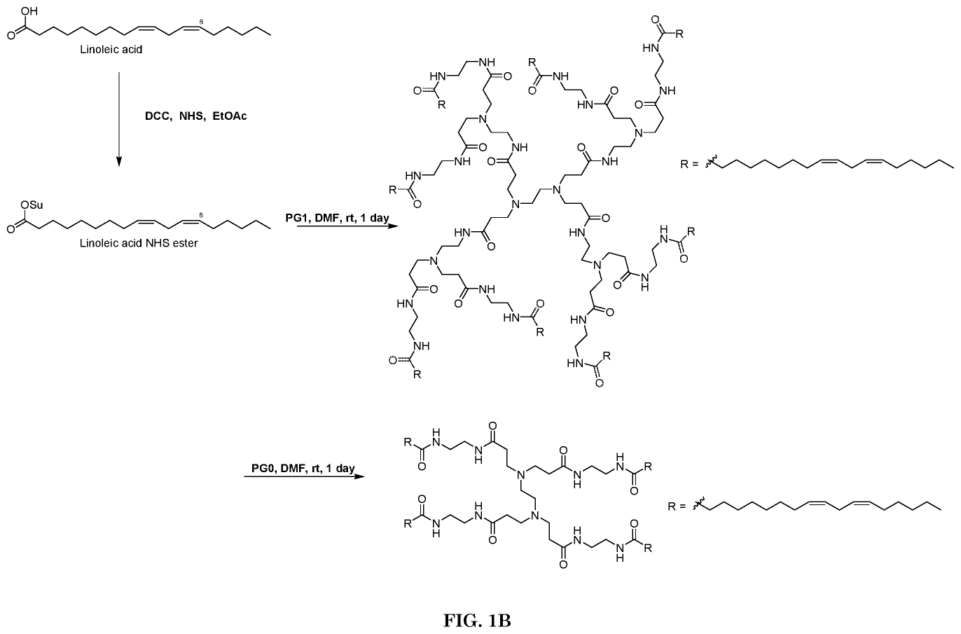

FIGS. 1A -1B illustrate modified dendrimers that include fatty acids in the terminal layer. FIG. 1A is a schematic drawing of the generation 1 modified PAMAM dendrimer (PAMAM-G₁, or PG1) and fatty acid side chains (R) that can be used for modification. In this figure, the fatty acid side chain R can be selected from an oleic acid, linoleic acid, arachidonic acid, or eicosapentaenoic acid. R can be selected from any one of C₄- C₂₈ fatty acids. PAMAM dendrimers with interior amide bonds exhibit greater biocompatibility than other dendrimer families, these bonding motifs are highly reminiscent of innate biological chemistry and endow PAMAM dendrimers with properties similar to that of globular proteins.

FIG. 1B illustrates synthesis of the PAMAM dendrimers modified with a fatty acid, linoleic acid. Referring to this figure, in the first step, under argon, linoleic acid and N-hydroxysuccinimide (NHS) were dissolved in 15 ml of the ethyl acetate (EtOAc). The solution was stirred at a room temperature (RT) followed by dropwise addition of dicyclohexylcarbodiimide (DCC; dissolved in 10 ml of EtOAc) into the solution to obtain a reaction ratio of 1:1:1 of linoleic acid: NHS: DCC. This mixture was further stirred at RT under argon for 12 hours. Dicyclohexylurea, a by-product, was removed by filtration, and the filtrate was concentrated under reduced pressure to yield the NHS ester. In the second step, the NHS ester was dissolved in 5 ml of dimethylformamide (DMF) and added dropwise to Gᵢ PAMAM dendrimer (PG1; top), or G₀ PAMAM dendrimer (PGO; bottom), dissolved in 1 ml of dimethyl sulfoxide (DMSO). The mixture was stirred for 24 hours under argon at RT. The reaction mixture was concentrated under reduced pressure in Genevac, and purified via flash chromatography on silica column with gradient elution from 100% CH₂Cl₂ to 75:22:3 CH₂CL₂/MeOH/NH₄OH_{aq} (by volume) over 40 minutes. The desired product was eluted at 50:7:1 CH₂CL₂/MeOH/NH₄OH_{aq}. Fractions containing the product were combined, dried under ramping high vacuum for 12 hours and stored at 4° C. until used.

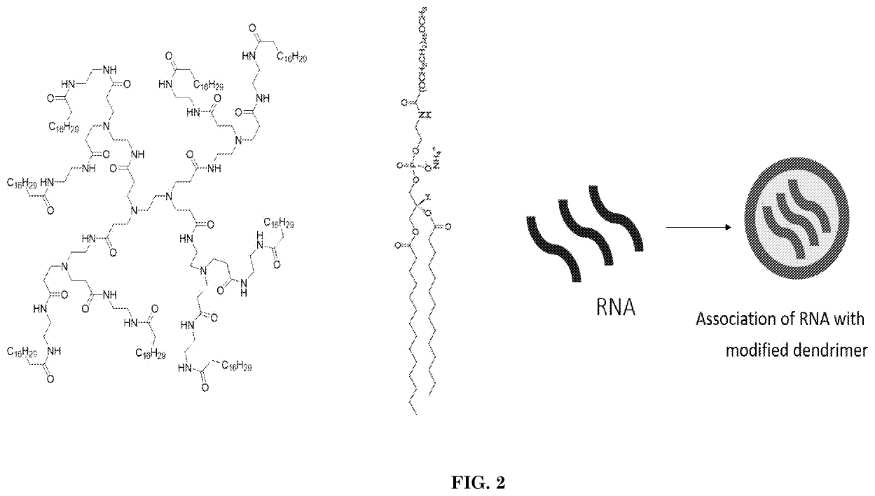

FIG. 2 illustrates a process for preparing a nanoparticle composition designed for improved self-assembly. Referring to FIG. 2, nanop articles were formulated via in-line mixing by using a microfluidic mixing device (Chen, D. et al., J. Am. Chem. Soc., 2012, 134 (16), pp 6948-6951, which is incorporated herein by reference as if fully set forth). The PG1 dendrimer modified to include linoleic acid tail in its terminal layer and 1,2-dimyristoyl-sn-glycero-3-phosphoethanolamine-N-[methoxy(polyethylene glycol)-2000] (27% w/w) were combined in ethanol. RNA was diluted with DNase/RNase-Free, endotoxin free distilled water and sterile citrate buffer to a final desired pH. Nanoparticle compositions described herein can be created at a pH in a range from 3.0 to 6.5. a low or high pH. It was observed that a low acidic pH, i.e., in a range from 3.0-3.5, creates a tighter binding of an RNA molecule to the carrier, an intermediate acidic pH, i.e., in a range from 3.5 to 4.5, creates an intermediate binding, and a high acidic pH, i.e., in a range of 4.5 to 6.5, creates a weaker binding.

The ethanol and citrate streams were loaded into gastight glass syringes and using a microfluidic mixing device, the ethanol and citrate streams were combined and mixed in a 1:3 volumetric flow rate ratio (combined total flowrate equal to 2.8 mL/min) to produce nanoparticles. Using glassware washed for 24 hours in 1.0 M NaOH for endotoxin removal and sterilized in a steam autoclave, or depyrogenated by heating at 2500C for 1 hour, nanop articles were dialyzed against sterile, endotoxin-free PBS using 20,000 molecular weight cutoff dialysis. Dialyzed nanoparticles were sterile filtered using 0.2 micron poly(ether sulfone) filters and characterized with a Zetasizer NanoZS machine (Malvern). The size distributions were characterized by a single peak with a low polydispersity index, indicating a relatively monodisperse size.

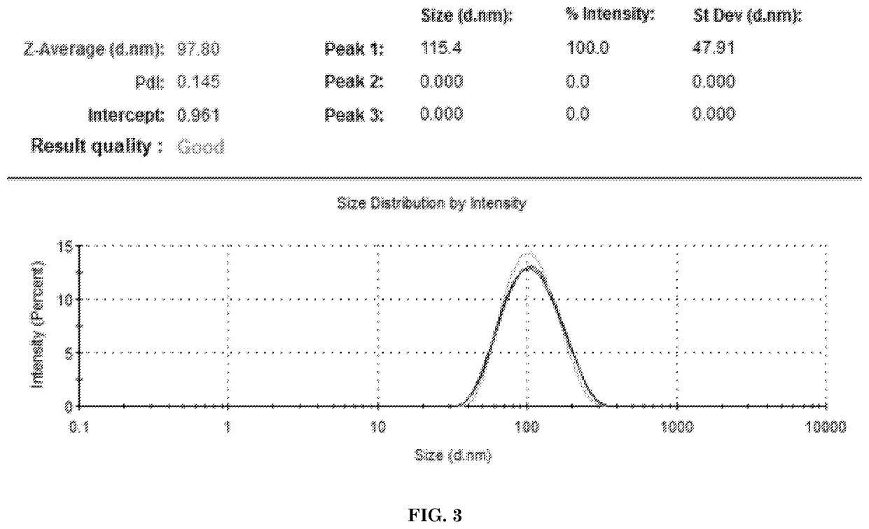

FIG. 3 illustrates illustrates particle size distribution of nanoparticles generated by mixture of PG1-linoleic acid modified dendrimer and SEAP replicon.

Referring to FIG. 3, the “Z average” of the nanoparticle composition as function of size was determined by dynamic light scattering (DLS). The “Z average” is the intensity weighted mean hydrodynamic size of the ensemble collection of particles measured by dynamic light scattering (DLS). Referring to this figure, high quality nanoparticles of uniform size were observed. The strongest intensity was observed for the nanop articles of 115.4 d.nm in size.

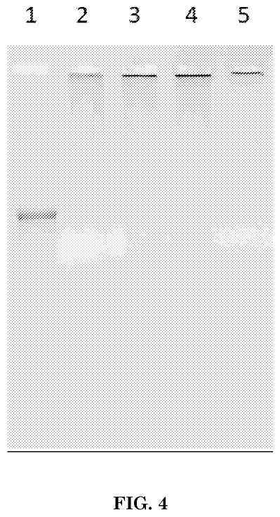

The concentration of RNA was determined by Nano Drop measurement (Thermo Scientific). Agarose gel electrophoresis was performed to evaluate the binding of modified dendrimer with RNA according to the known method (Tang et al., 2019, Asian J. Pharm. Sci., 15:55, which is incorporated herein by reference as if fully set forth. FIG. 4 is a photograph of the agarose gel demonstrating the binding of the modified dendrimer with RNA. Referring to FIG. 4, lane 1 contained the unformulated SEAP replicon, lane 2 contained the product of formulation of the PG1-oleic acid dendrimer and SEAP replicon, lane 3 contained the product of formulation of the PG1-linoleic acid and SEAP replicon, lane 4 contained the product of formulation of the PG1-arachidonic acid and SEAP replicon, and lane 5 contained the product of formulation of the PG1-eicosapentaenoic acid (EPA) and SEAP replicon. Before loading, the samples were incubated with formaldehyde loading dye, denatured for 10 min at 65 ° C. and cooled to room temperature. The gel was run at 90 V and gel images were taken on a Syngene G Box imaging system (Syngene, USA). For RNA detection, the gel was stained with ethidium bromide. Referring to FIG. 4, the lower band corresponds to the small size free RNA (lane 1) and the top bands represent the large size nanoparticles formed by binding of the RNA to the dendrimer carriers: PG1-oleic acid (lane 2), PG1-Linoleic acid (lane 3), PG1-Arachidonic acid (lane 4), and PG1-eicosapentaenoic acid (EPA; lane 5).

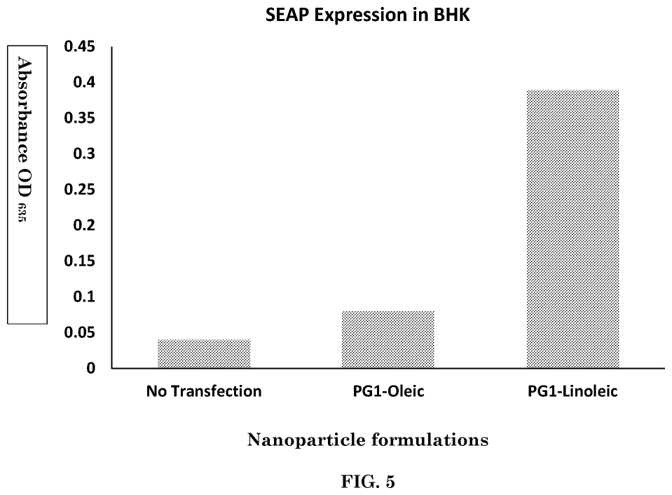

To test formulations of modified dendrimer, the secreted embryonic alkaline phosphatase SEAP reporter system was used. For in vitro tests, 90% confluent 12 well of the baby hamster kidney were used. The treatment was performed by replacing the media with 1:1 PBS/Optimem followed by treatment with nanoparticle in PBS. After the treatment, BHK cells were incubated at 37° C. and 5% CO₂. After 12 hours, cell culture medium was collected and assayed for SEAP using the InvivoGen QUANTI-Blue™ detection system (San Diego, Calif., USA), according to the manufacturer's protocol. Briefly, 50 μL of the cell culture medium was added to 150 μL of the QUANTI-Blue™ solution and incubated at 37° C. for 10 minutes. The Optical Density (OD) was measured at 620-655 nm using a microplate reader. FIG. 5 illustrates the SEAP expression of nanoparticle formulations using PG1-oleic acids and PG1-linoleic acids based on optical density compared to the negative control, where cells were treated with buffer containing no nanop articles (No transfection). The highest level of SEAP expression was observed for the PG1-linoleic acid nanoparticle formulation.

### Example 2. Changing the Rate of the Nucleic Acid Payload Release in Cytoplasm

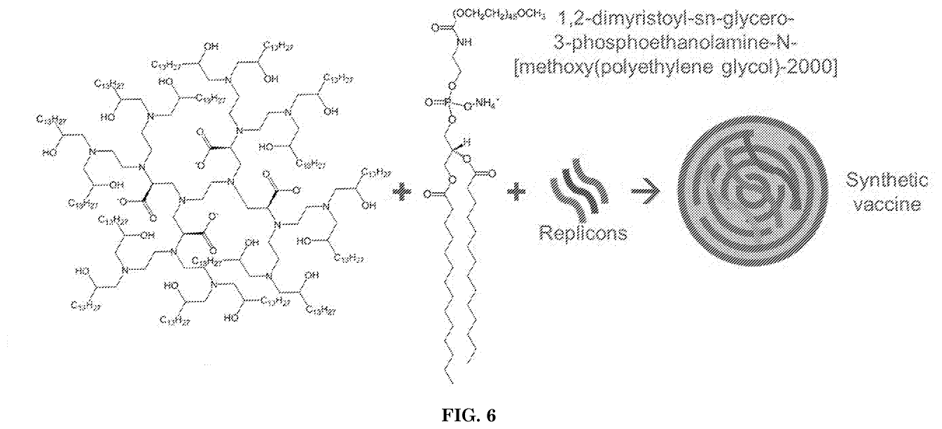

To change the rate of nucleic acid payload release in the cytoplasm, nanoparticles were formulated at different pH values. Slow release stable particles that take longer to release their nucleic acid payloads inside the cell are formed by formulating nanop articles at lower pH values, such as pH 3.0. The amine groups become protonated, increasing their charge density, which forms more electrostatic associations with the nucleic acid payloads. This results in binding, which slows down the ultimate release of the nucleic acid. Faster release is achieved by formulating nanoparticles at a higher pH, such as 5.0. With fewer H⁺ ions available, there is less protonation of the amine groups and fewer electrostatic associations between the nucleic acid and the delivery material. This reduced electrostatic association results in weaker binding, and faster release of the nucleic acid payload inside the cell. FIG. 6 illustrates the process of preparing a synthetic vaccine that includes a modified dendrimer, 1,2 dimyristoyl-sn-glycero-3-phosphoethanlomine-N-[methoxy(polyethylene glycol)-2000], and replicons. Nanoparticles were formulated via in-line mixing by the use of a microfluidic mixing device (Chen, D. et al., J. Am. Chem. Soc., 2012, 134 (16), pp 6948-6951, which is incorporated herein by reference as if fully set forth). The modified dendrimer and 1,2-dimyristoyl-sn-glycero-3-phosphoethanolamine-N-[methoxy(polyethylene glycol)-2000] (Avanti Polar Lipids)) are combined in ethanol. RNA was diluted with DNase/RNase-Free, endotoxin free distilled water and sterile citrate buffer to a final desired pH. It was observed that a low pH, such as pH 3.0, creates tighter binding, and a higher pH, such as pH 6.5, creates weaker binding. The ethanol and citrate streams were loaded into gastight glass syringes and using a microfluidic mixing device, the ethanol and citrate streams were combined and mixed in a 1:3 volumetric flow rate ratio (combined total flowrate equal to 5.3 mL/min) to produce nanoparticles. Using glassware washed for 24 hours in 1.0 M NaOH for endotoxin removal and sterilized in a steam autoclave, or depyrogenated by heating at 250° C. for 1 hour, nanop articles are dialyzed against sterile, endotoxin-free PBS using 20,000 molecular weight cutoff dialysis. Dialyzed nanoparticles were sterile filtered using 0.2 micron poly(ether sulfone) filters.

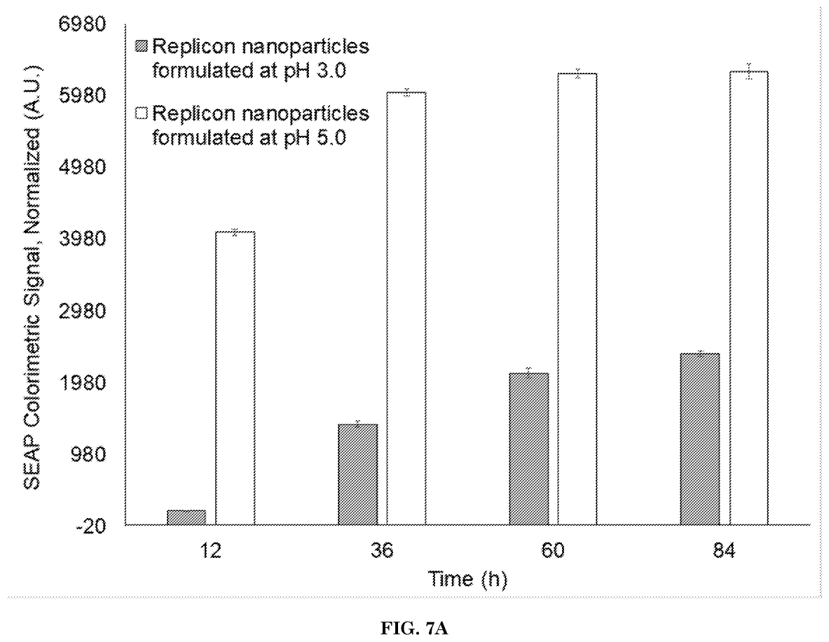

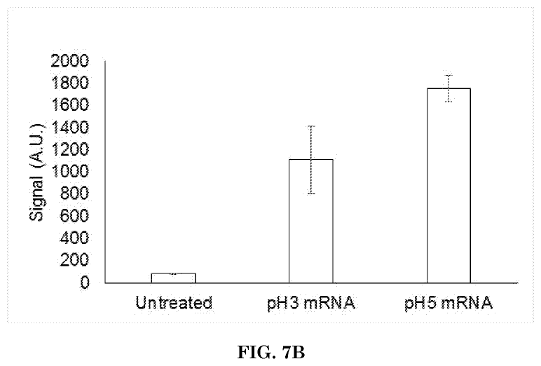

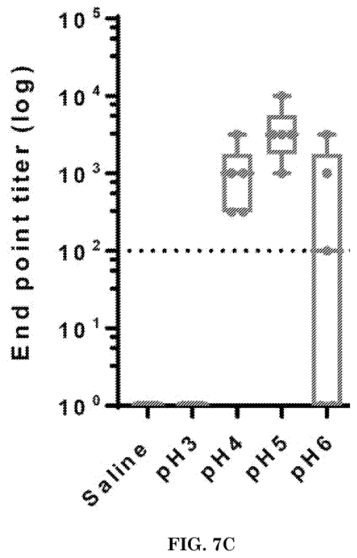

To test formulations of modified dendrimer, the secreted embryonic alkaline phosphatase SEAP reporter system was used. FIGS. 7A-7C illustrate the effect of pH during formulation of the nanop article composition on its stability and the replicon release time.

For in vitro tests, the baby hamster kidney (BHK) cells in the 96 well plate format were used. The BHK cells were treated with the modified dendrimer nanoparticles and incubated at 37° C. and 5% CO₂. After 12, 36, 60 or 84 hours, cell culture medium was collected and assayed for SEAP using the InvivoGen QUANTI-Blue™ detection system (San Diego, Calif., USA), according to the manufacturer's protocol. Briefly, 20 μL of the cell culture medium was added to 180 μL of the QUANTI-Blue™ solution and incubated at 37° C. for 60 minutes. The Optical Density (OD) was measured at 620-655 nm using a microplate reader. The OD reading was normalized to the OD reading of samples from cells that were not treated with nanoparticles. The result of the normalization was the SEAP colorimetric signal, normalized (A.U.), where A.U. means arbitrary units. FIG. 7A illustrates a replicon mRNA expressing SEAP that was synthesized and formulated into modified dendrimer nanoparticles at a pH of 3.0 or 5.0. Referring to FIG. 7A, it was observed that SEAP expression was higher with the pH 5.0 formulation, as compared to pH 3.0 because replicon mRNA was released sooner from the pH 5.0 formulation due to weaker binding. The pH 3.0 formulation has its replicon mRNA more tightly bound, so the magnitude to SEAP signal over time was also lower, as the replicon release was slower and required more time. In this way, controlled release can be achieved by altering the strength of binding via formulation pH.

It was observed that a pH higher than 3 when formulating the delivery material with nucleic acids resulted in the creation of nanoparticles that are more efficacious in vivo. Typically, the nanoparticles are formulated at pH 3 (Khan et al. Angew Chem Int Ed Engl. 2014 Dec. 22; 53(52): 14397-14401; Khan et al, Nano Lett. 2015 May 13; 15(5): 3008-3016, and Chahal et al., Proc Natl Acad Sci USA. 2016 Jul. 19; 113(29):E4133-42, all of which are incorporated herein by reference as if fully set forth). By using a pH higher than 3.0 herein, such as pH 4.0 or pH 5.0, the unanticipated effect of dramatically boosting the in vivo performance of nanoparticles was observed. The in vitro results did not predict dramatic magnitude of performance improvement in vivo and this is an important specification for an actual nanoparticle product.

### Example 3: RNA Release Rate from Nanoparticle Influences T Cell Response In Vivo

To change the kinetics of the antigen-specific immune responses in vivo, nanoparticles were formulated at different pH values, which alter the controlled release and expression of the formulated RNA. Slow release stable particles that take longer to release their nucleic acid payloads inside the cell were formed by formulating nanoparticles at lower pH values, such as 3.0. The amine groups become protonated, increasing their charge density, which forms more electrostatic associations with the nucleic acid payloads. This results in binding, which slows down the ultimate release of the nucleic acid. This slower release results a reduced amount of RNA expression at earlier time points in vivo. Faster release in vivo is achieved by formulating nanoparticles at a higher pH, such as 5.0. With fewer H+ions available, there is less protonation of the amine groups and fewer electrostatic associations between the nucleic acid and the delivery material. This reduced electrostatic association results in weaker binding, and faster release of the nucleic acid payload inside the cell. This results in stronger RNA expression earlier.

To test formulations of modified dendrimer, the secreted embryonic alkaline phosphatase SEAP reporter system was used. Nanop articles were formulated via in-line mixing as described in Example 2 herein. FIG. 7B illustrates a conventional mRNA expressing SEAP that was synthesized and formulated into modified dendrimer nanop articles at a pH of 3.0 or 5.0. For in vivo tests, mice were vaccinated with nanoparticles at a dose of 100 ng of SEAP mRNA, and 5 days later, serum was collected from the mice. The amount of quantified using the Invitrogen NovaBright™ Phospha-Light™ EXP Assay kits for SEAP detection according to the manufacturer's protocol. The amount of SEAP in the mouse serum samples are reported in Arbitrary Units (A.U.). Error bars are±S.E.M. Referring to FIG. 7B, it was observed that the day 5 SEAP amount was higher with the pH 5.0 formulation, as compared to pH 3.0 because the mRNA was released sooner from the pH 5.0 formulation due to weaker binding. The pH 3.0 formulation has its mRNA more tightly bound with a slower release rate. In this way, controlled release can be achieved by altering the strength of binding via formulation pH.

FIG. 7C illustrates the effect of increasing the pH during the nanoparticle manufacturing process on the in vivo performance of the nanoparticles. In particular, this figure shows antibody responses following vaccination with nanoparticles formulated at different pH. Nanoparticles carrying RNA replicons expressing the Venezuelan Equine Encephalitis E1/E2 polypeptide were administered via intramuscular injections into mice as a vaccine. To form the nanoparticles, a microfluidic mixing device was used (Chen, D. et al., J. Am. Chem. Soc., 2012, 134 (16), pp 6948-6951). The modified dendrimer (PAMAM generation 1, core substituted with C₁₅ alkyl groups) and 1,2-dimyristoyl-sn-glycero-3-phosphoethanolamine-N-[methoxy (polyethylene glycol)-2000] (Avanti Polar Lipids)) were combined in ethanol. RNA was diluted with DNase/RNase-Free, endotoxin free distilled water and sterile citrate buffer to a final pH of 3.0, 4.0, 5.0 or 6.0. The ethanol and citrate streams were loaded into gastight glass syringes and using a microfluidic mixing device, the ethanol and citrate streams were combined and mixed in a 1:3 volumetric flow rate ratio (combined total flowrate equal to 5.3 mL/min) to produce nanoparticles. Using glassware washed for 24 hours in 1.0 M NaOH for endotoxin removal and sterilized in a steam autoclave, or depyrogenated by heating at 250° C. for 1 hour, nanoparticles were dialyzed against sterile, endotoxin-free PBS using 20,000 molecular weight cutoff dialysis. Dialyzed nanoparticles were sterile filtered using 0.2 micron poly(ether sulfone) filters. After vaccination, serum was collected and tested for an antibody response to the E 1/E2 polyprotein via ELISA and end point titer determination. Referring to FIG. 7C, nanoparticles formulated at pH 3.0 showed little to no antibody response to the E1/E2 polyprotein. Surprisingly, the nanoparticles formulated at pH 4.0 and 5.0 showed extremely high antibody responses to the E1/E2 polyprotein. At pH 6, the antibody response lacked reproducibility across all animals and was very variable. Saline was used as a negative control and showed no antibody response.

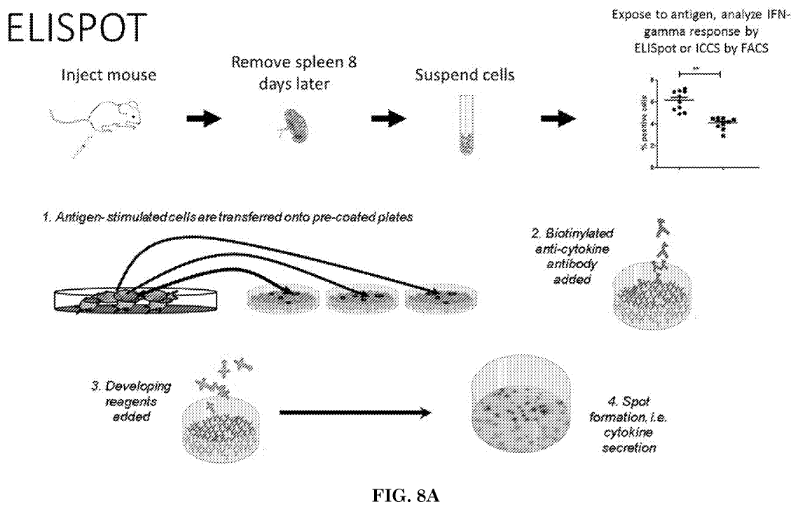

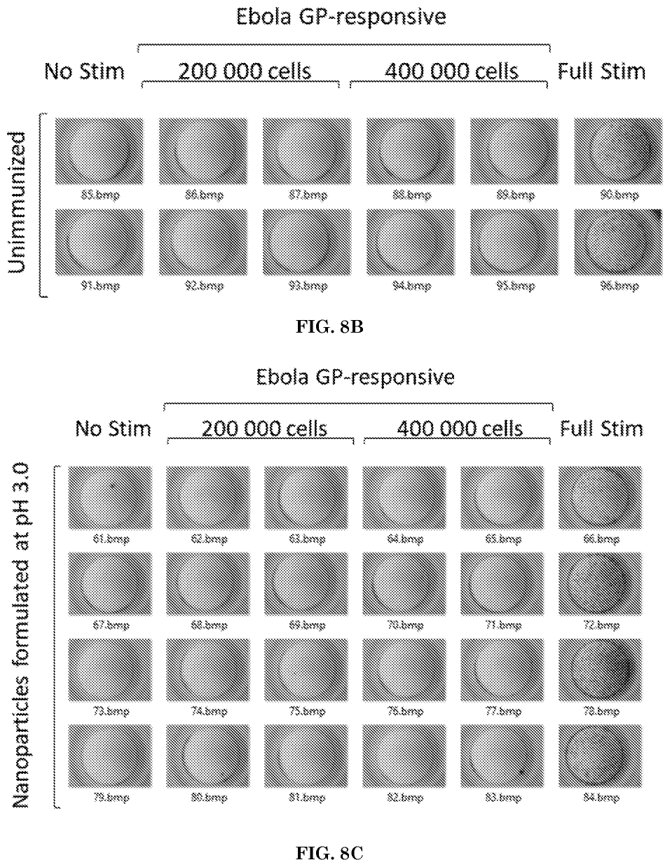

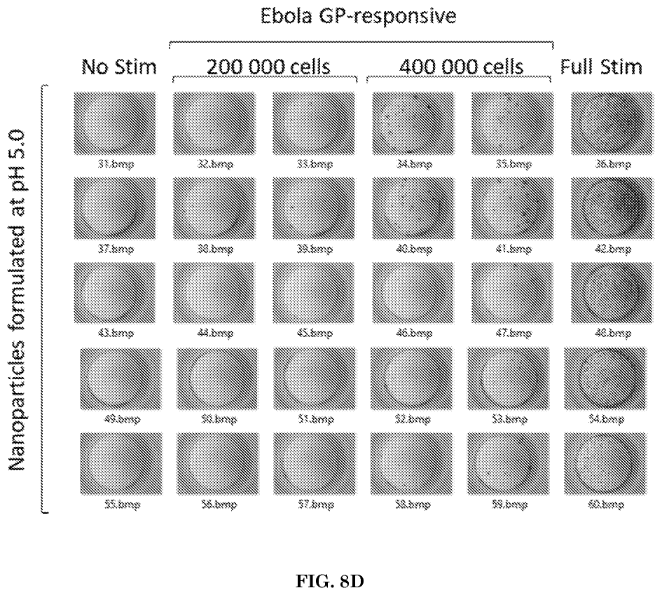

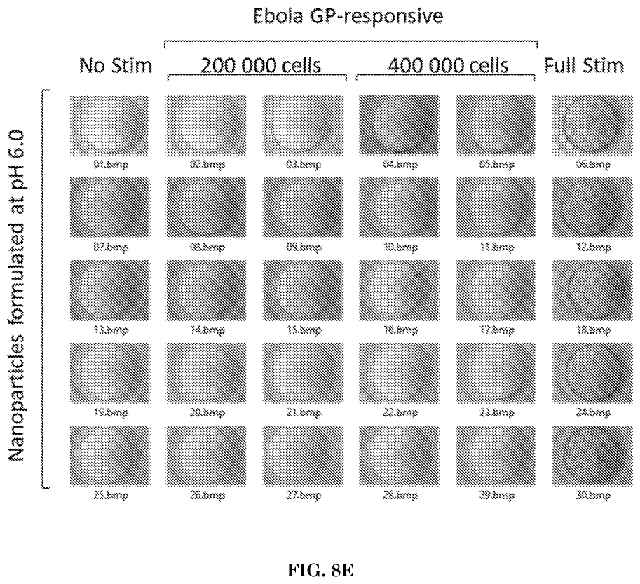

FIGS. 8A-8E illustrate the effect of formulation pH on vaccine performance in vivo. FIG. 8A illustrates steps of the ELISPOT test used to assess the T cell response following vaccination with nanoparticles formulated at pH 3.0, 5.0 and 6.0 and containing replicons expressing Ebola GP. For this test, animals (n=5) were vaccinated with the nanoparticles. The T cell response was analyzed by ELISPOT. In the test, the antigen-stimulated cells were transferred onto the pre-coated plates (1), biotinylated cytokine antibody was added (2), developing reagents were added (3), and spot formation, i.e., cytokine secretion (4) was observed. Specifically, spleens of the vaccinated animals were removed 8 days after vaccination and placed into 5 mL of cRPMI medium in 15 mL conical vials on ice. Spleens were mashed through a 70 μm mesh in 10 cm dishes to break down connective tissue and create a single cell suspension. To wash, the cell suspension was diluted with phosphate buffered saline and centrifuged to produce a cell pellet. The pellet was loosened, treated with red blood cell lysis buffer. After treatment, cells were again pelleted and then resuspended in cRPMI. Using the cell suspension and the BD Biosciences ELISPOT kit, the ELISPOT assay was performed according to the manufacturer's protocol. Anti-CD49d and anti-CD28 antibodies were used. For stimulation tests, a WE15 peptide was used (Ebola GP-responsive). No peptide stimulation was negative controls (No Stimulus; No Stim) and fully activated cells served as positive controls (Full stimulus; Full Stim).

FIGS. 8B-8E illustrate the ELISPOT test results. In these figures, for each pH condition, each row corresponds to an individual mouse. No stimulus (No Stim) and full stimulus (Full Stim) are negative and positive controls, respectively, that were run as singles. FIG. 8B illustrates plates prepared with unimmunized control cells. No spots were observed in plates containing unimmunized cells. Test cases (Ebola GP-responsive) were done in technical duplicate and were run as duplicates. FIG. 8C illustrates data for nanop articles formulated at pH 3.0. Referring to this figure, due to the slow release of the “pH 3.0” nanoparticle formulations, no Ebola GP-specific T cell response (i.e., little to no spots) was observed at the 8 day time point (too early to generate a response). FIG. 8D illustrates data for nanoparticles formulated at pH 5.0. As evident the multiple dark spots in the circular wells, the “pH 5.0” nanoparticle formulation did show a strong T cell response, as the replicon payload was less tightly bound and able to be released and expressed sooner than the payload formulated at pH 3.0. FIG. 8E illustrates data for nanoparticles formulated at pH 6.0. The “pH 6.0” nanoparticles showed a weak signal (i.e., little to no spots) due to the premature release of the replicon payload due to nanop article instability. There was not enough positive charge to form cohesive nanop articles that were able to survive long enough to deliver the replicon payload for mRNA replicon expression.

### Example 4. Nanoparticle Compositions Comprising the STING Activator

To improve performance of vaccines, a small molecule agonist of the STING protein (also referred to herein as STING agonist or STING activator) is mixed with a modified dendrimer and nucleic acid to form a nucleic acid nanoparticle. Small molecule agonists of the STING protein may be modified to incorporate a hydrophobic moiety, such as alkyl, alkenyl, alkynyl, saturated or unsaturated fluorinated carbons, The hydrophobic moiety is optionally included to assist with nanoparticle self-assembly by anchoring the STING agonist to the other hydrophobic moieties present in the nanop article formulation (for example, those of a modified dendrimer or lipid-anchored PEG). FIG. 9 illustrates an exemplary STING activator, a cyclic guanosine monophosphate—adenosine monophosphate (2′3′-cGAMP), in which H groups of the primary amine (NH₂) are substituted with hydrophobic R functional groups. The STING activator or derivative can be mixed with any one of the modified dendrimers described herein.

### Example 5. Tracking of the Nanoparticle Compositions

To facilitate tracking of the delivery material in vitro and in vivo, heterogeneous multilayer modified dendrimers can have cores containing stable isotopes of carbon (C) or nitrogen (N), such as ¹³C or ¹⁵N. FIG. 10 illustrates structures of one-layer modified dendrimers with the cores containing stable isotopes of nitrogen (¹⁵N; top structures) and carbon (¹³C; bottom structures). When the modified dendrimers are formulated into nanoparticles with nucleic acids, e.g., replicon mRNA, they can be tracked in vitro and in vivo post-administration by any known technique, for example, mass spectroscopy or nuclear magnetic resonance imaging. The inclusion of the stable isotopes makes identification of the delivery molecules easier since they become different from the abundant ¹²C and ¹⁴N isotopes that are dominantly found in tissues. Tracking can be useful for identifying biodistribution, material clearance and molecular stability of nanoparticles post-administration, and related issues.

### Example 6. Control Nanoparticle Vaccine Biophysical Characteristics and Performance Through Amphiphilic Polymer

Key performance and biophysical parameters for nanoparticle are controlled by the mass percentage of the amphiphilic PEG in the nanoparticle composition. In the following examples, nanoparticles were formulated via in-line mixing by using of a microfluidic mixing device as described in Example 2 herein. The PG1.C15 modified dendrimer and amphiphilic polymer 1,2-dimyristoyl-sn-glycero-3-phosphoethanolamine-N-[methoxy(polyethylene glycol)-2000] (Avanti Polar Lipids, an amphiphilic PEG)) were combined in ethanol. In the PG1.C15 modified dendrimer, “PG1” refers to the core consisting of the first generation poly(amido amine) dendrimer, and “C15” refers to the length of the substituted alkyl chains. Depending on the preparation, a different amount (mass percentage) of amphiphilic PEG was used. Replicon RNA was diluted with DNase/RNase-free, endotoxin free distilled water and sterile pH 3.0 citrate buffer to a final citrate concentration of 10 mM. The ethanol and citrate streams were loaded into gastight glass syringes and using a microfluidic mixing device, the ethanol and citrate streams were combined and mixed in a 1:3 volumetric flow rate ratio, respective, (combined total flowrate equal to 5.3 mL/min) to produce nanop articles. Nanop articles were immediately diluted at a 1:100 dilution factor with sterile DNAse/RNAse free phosphate buffered saline to dilute the ethanol and prevent its further action as a solvent. To measure nanop article size and zeta potential, the diluted preparation was loaded into Malvern DTS1070 folded capillary cells and measured using a Malvern Zetasizer ZS according to the manufacturer protocols. To measure nanoparticle concentration, the diluted preparation was tested using a Malvern NanoSight NS300 according to the manufacturer's protocol.

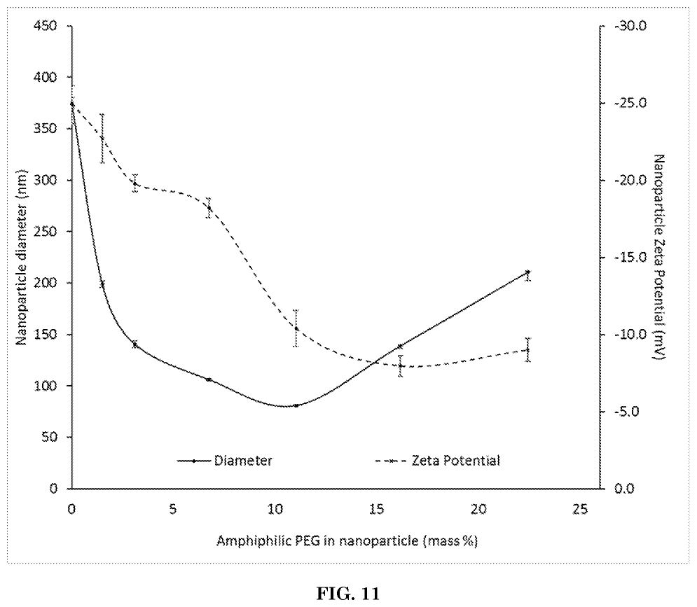

Regarding minimization of particle size and aggregation, it is important to ensure the nanoparticles are small enough to easily enter cells, and to ensure they do not aggregate or form larger particles that can sediment or not enter cells easily. FIG. 11 illustrates the effect of PEG on the size and aggregation ability of the nanoparticle composition. Referring to FIG. 11, the solid line shows the diameter of the nanoparticle based on mass % of the amphiphilic PEG per nanoparticle and the dashed line shows zeta potential of the nanoparticles based on mass % of the amphiphilic PEG per nanoparticle. It was observed that by controlling the amount of the amphiphilic polymer in the nanoparticle composition, both the size and aggregation ability of the nanoparticle composition can be controlled. The lower the mass percentage of amphiphilic PEG, the larger the nanoparticles become, and the larger the absolute value of the zeta potential. While larger particles can sediment faster due to increased weight, altering colloidal stability, the larger zeta potential magnitude helps prevent aggregation. However, larger particles can be harder for cellular uptake. It is possible to maintain a sufficiently high magnitude of zeta potential while shrinking nanoparticle diameter. Preferably, the mass percentage of amphiphilic PEG should be greater or equal to 1.0% (w/w), or lesser or equal to 22% (w/w) per nanoparticle composition, which ensures nanoparticle diameter remain less or equal to 200 nm, and thus, able to fully pass through a sterilization filter with 0.2 μm pore size.

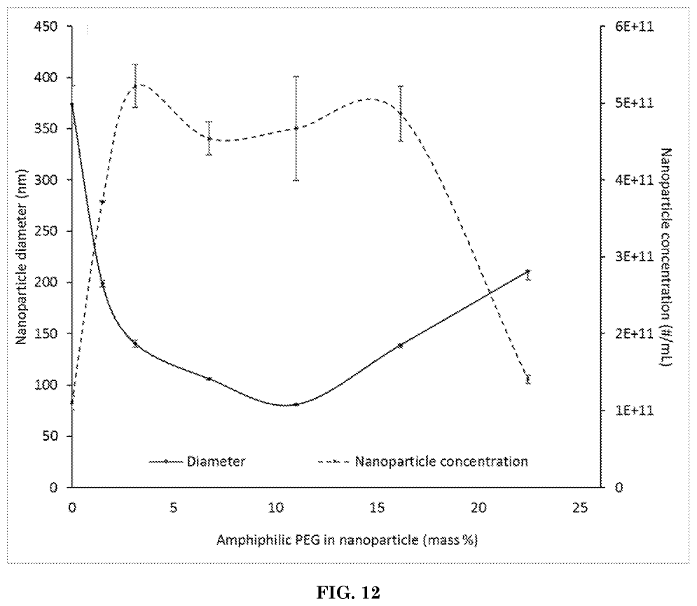

The distribution of payload across smaller and more numerous particles is important to maximize uptake by many cells. FIG. 12 illustrates the effect of incorporation of amphiphilic PEG molecules on the diameter and concentration of the particles in the nanoparticle composition. Referring to FIG. 12, the solid line shows the diameter of the nanoparticle based on mass % of the amphiphilic PEG and the dashed line shows the nanoparticle concentration based on mass % of the amphiphilic PEG per nanoparticle. This figure shows that the incorporation of the PEG molecules affects the distribution of the particles across recipient cells. For example, PEG affects the distribution of payload across smaller and more numerous particles. If the payload is sequestered into fewer, larger particles, there fewer cells can experience uptake. More particles can be taken up by more cells. Thus, by altering the amount of amphiphilic PEG mass % in the nanoparticle composition, it was possible to create a larger number of particles while keeping the nanoparticle diameter small enough to facilitate easier cellular uptake. This is particularly useful for replicon RNA payloads, where, due to self-replication of the replicon mRNA, fewer copies of replicons are required per cells, and the focus is instead on maximizing the number of cells that receive fewer copies of the replicon. Mass % of the amphiphilic polymer per nanoparticle composition can be within a range of 1.0% (w/w) to 22% (w/w), as it helps maintain nanoparticle diameters lesser or equal 200 nm which allows all particles to pass through a sterilization filter with 0.2 μm pore sizes without loss.

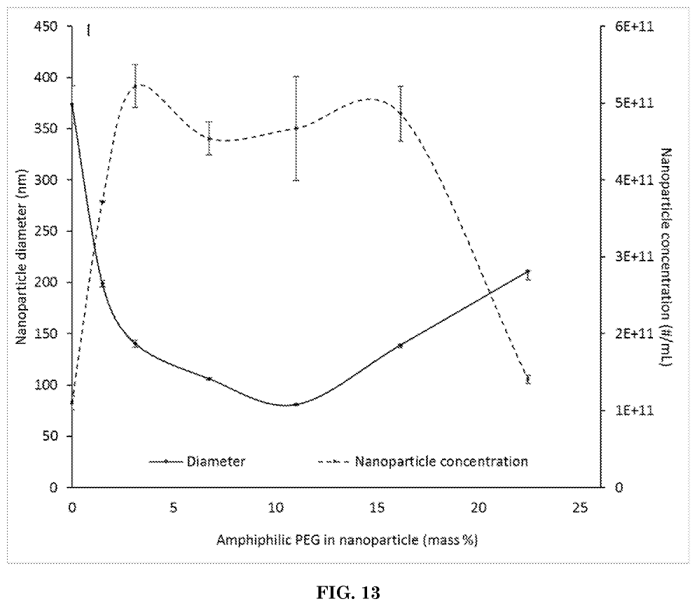

It follows that it is important to control the absolute number of RNA molecules per nanop article. FIG. 13 illustrates the effect of incorporation of amphiphilic PEG molecules on the number of RNA molecules per nanoparticle. Referring to FIG. 13, the solid line shows the diameter of the nanoparticle based on mass % of the amphiphilic PEG and the dashed line shows the nanoparticle concentration based on mass % of the amphiphilic PEG. This effect can be used for controlling the number of RNA molecules per nanoparticle. This is important to control because for certain payloads, such as replicon mRNA, fewer RNA copies are acceptable since they can self-replicate. However, for conventional RNA payloads that do not replicate, such as conventional mRNA, more copies per particle would be advantageous to improve and increase expression. A small mass % of amphiphilic polymer (amphiphilic PEG) creates larger diameter nanoparticles that carry more RNA molecules per particle. Increasing the mass % of the amphiphilic PEG decreases nanop article size and concomitantly decreases the amount of RNA molecules per nanoparticle. However, when amphiphilic PEG contents a certain point, such as less than 15%, the particle diameter and copies per particle begin to increase with increasing amphiphilic PEG %. Thus, one can tune the payload packing while simultaneously controlling for other parameters, such as zeta potential to ensure particles stay separate and do not aggregate. At higher amphiphilic PEG mass %, the zeta potential magnitude drops, which can lead to more particle aggregation. Knowing this parabolic trend, one can tune the systems using amphiphilic PEG content to best suit the application. Mass % of the amphiphilic polymer per nanoparticle composition can be within a range of 1.0% (w/w) to 22% (w/w), as it helps maintain nanop article diameters less or equal to 200 nm which allows all particles to pass through a sterilization filter with 0.2 μm pore sizes without loss.

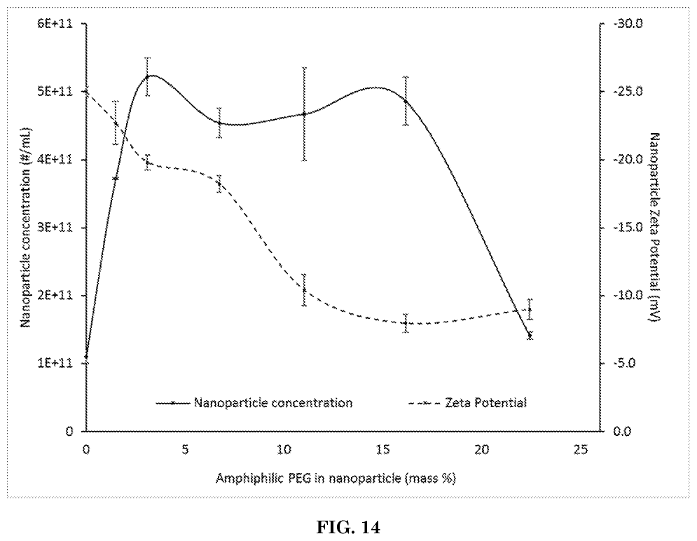

Tuning nucleic acid payload distribution to cells is also important and can be controlled by the mass % of amphiphilic PEG in the nanoparticle composition. FIG. 14 illustrates the effect of incorporation of amphiphilic PEG molecules on the ability to increase the degree of the electrostatic repulsion between nanoparticles and their dispersion. Referring to FIG. 14, the solid line shows the nanop article concentration based on mass % of the amphiphilic PEG per nanoparticle and the dashed line shows zeta potential of the nanoparticles based on mass % of the amphiphilic PEG per nanoparticle. This can be used for tuning nucleic acid payload distribution to cells. For some applications, maximizing the spread of nanoparticles after administration is beneficial, to ensure the greatest number of cells can take up the nanoparticles' payloads. The number of particles that are formed thermodynamically can be increased by altering the free energy of the nanoparticles through an increasing mass % of amphiphilic PEG. It becomes more energetically favorable to self-assemble into more numerous, smaller particles. However, as the amphiphilic PEG mass % increase, the zeta potential decreases, which can lead to more particle aggregation, preventing their broader spread to and uptake to more cells. Mass % of the amphiphilic polymer per nanoparticle composition can be within a range of 1.0% (w/w) to 22% (w/w), as it helps maintain nanoparticle diameters less or equal to 200 nm which allows all particles to pass through a sterilization filter with 0.2 μm pore sizes without loss.

This above study systematically evaluated the effect of polyethylene glycol (PEG) concentration on a nanoparticle's size, zeta potential, aggregation etc. The effect of PEG concentration on a nanoparticle's biophysical properties depends on the physicochemical properties of the particular modified dendrimer in the composition. The modified dendrimer used in the above composition was PG1C15. For a modified dendrimer substituted with fatty acid tails, or heterogeneous dendrimers with different physicochemical properties, the optimal mass % of the amphiphilic polymer per nanoparticle composition may be different. The mass percent can be within a range of 1.0% (w/w) to 40% (w/w) to help maintain nanoparticle diameters less or equal to 200 nm which allows all particles to pass through a sterilization filter with 0.2 μm pore sizes without loss while maintaining other desired biophysical properties.

### Example 7. Forming Modified Dendrimer Nanoparticles with Drugs that Carry a Full or Partial Negative Charge

Drugs that contain negative or partially negative charges are formulated with modified dendrimers to form nanop articles via the electrostatic association of the negative charge with the positive charge of the protonated amine groups in the modified dendrimer. Drugs can, for example, contain phosphate, phosphonate, phosphinate or sulfone functional groups.

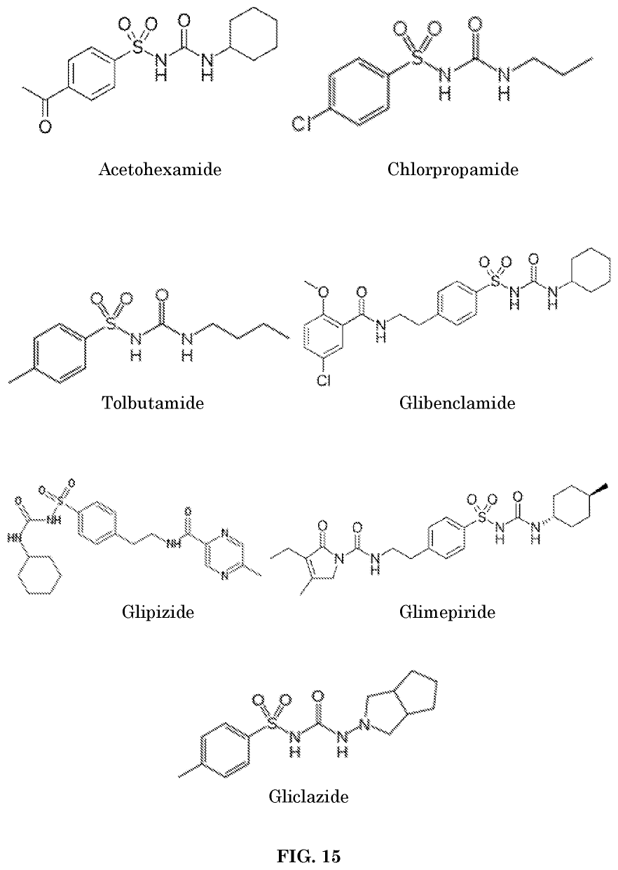

FIG. 15 shows exemplary sulfonylurea drugs acetohexamide, chlorpropamide, tolbutamide, glibenclamide, glipizide, glimepiride, and gliclazide that can be included in the nanoparticle compositions herein.

Examples of phosphate-containing nucleotide analogs include drugs used in cancer and viral chemotherapy, such as purine and pyrimidine nucleoside analogs, Arabinosylcytosine (ara-C), Ara-C monophosphate (ara CMP), azidothymidine (AZT), AZT monophosphate (AZTMP), 2′3′-dideoxycytidine (ddCD), cyclic adenoside monophosphate (cAMP), tenofovir, or adefovir.

To demonstrate the ability to form stable nanoparticles with a drug, the PG1.C12 modified dendrimer (PAMAM G1 core modified with C₁₂ alkyl chains) was formulated with acetohexamide to form nanoparticles. The sulfone group creates a partial negative charge, which is used to find to the protonated modified dendrimer that has a positive charge.

A 3 mg/mL solution of acetohexamide in 200 proof ethanol was prepared by sonicating the mixture for 30 minutes until it was dissolved. Then, 83.3 μL o the 3 mg/mL acetohexamide solution as diluted to a final volume of 375 μL with pH 7.4 phosphate buffered saline as the diluent. This was loaded into a 1 mL gastight syringe.

A solution containing PG1.C12 modified dendrimer, cholesterol and 1,2-dimyristoyl-sn-glycero-3-phosphoethanolamine-N-[methoxy (polyethylene glycol)-2000] (ammonium salt) was next prepared. 86.5 μL of a 13 mg/mL PG1.C12 in ethanol solution, 4.7 μL of a 5 mg/mL cholesterol in ethanol solution, a 5.8 μL of a 20 mg/mL 1,2-dimyristoyl-sn-glycero-3-phosphoethanolamine-N-[methoxy (polyethylene glycol)-2000] in ethanol solution, and 28 μL of 200 proof ethanol were combined and loaded into a 1 mL gastight syringe.

The two solutions were combined using a microfluidic mixing device as previously described (Chen, D., et al, J Am Chem Soc. 2012 Apr. 25; 134(16):6948-51). The acetohexamide solution's flow rate was 3976 μL/min and the PG1.C15-containing solution's flow rate was 1325.3 μL/min.

The resulting nanop articles were dialyzed against 1 L of pH 7.4 phosphate buffered saline overnight and the particle size distribution was determined by dynamic light scattering using a Malvern Zetasizer ZS. The presence of acetohexamide in the dialyzed nanop article solution was confirmed by high pressure liquid chromatography. For example, using a 50% acetonitrile solution of the nanop articles can be chromatographed using Lichrosorb RP-8 reverse phase column and the mobile phase composed 0.2% AcOH-acetonitrile (1:1) similar to a published protocol (Takagishi, Y., Sato, K., Tomita, K., and Sakamoto, T., 1979, High-Performance Liquid Chromatographic Determination of Acetohexamide and Its Metabolite, Hydroxyhexamide, Yakugaku Zasshi, 99(9), 961-963, which is incorporated herein by reference as if fully set forth).

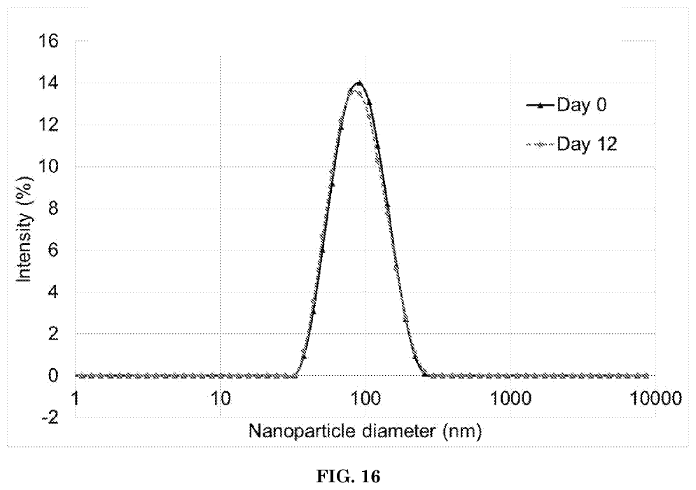

The stability of the nanoparticles was determined by comparing the particle size distribution 12 days after formulation and storage at 4° C. with the particle size distribution measured immediately after the overnight dialysis. FIG. 16 illustrates the stability of the PG1.C12 modified dendrimer nanoparticles containing acetohexamide assessed by the particle size distribution measured by dynamic light scattering followed production (Day 0; solid line) and 12 days after formulation and storage at 4° C. (Day 12; dashed line). It was observed that the particle size distributions were the same, indicating stability under these storage conditions.

### Example 8. Design and In Vivo Expression of STING mRNA as a Genetic Adjuvant

Stimulator of interferon genes (STING) is an endoplasmic reticulum protein which mediates cytosolic DNA-induced signaling events. STING is a signaling adaptor protein, that potentiates the phosphorylation (and thus activation) of transcription factors that activate Type I interferon (IFN) responses when stimulated by the presence of cytosolic DNA-derived metabolites. As a critical anti-viral signaling component of the innate immune system, artificially controlling intracellular STING activity has multiple applications in the treatment of human and animal disease, particularly in pathologies involving the innate or adaptive immune systems.

Enhancing STING activity can result in increased immune responses via Type I IFN activity, which can be exploited, for example, to increase anti-tumor immunity, or to improve the potency of conventional vaccines. To this end, many small-molecule ligands of STING have been developed as potential ‘adjuvants’ to stimulate the pathway concurrently with vaccination, or simply as a strategy to globally enhance innate immune signaling to increase anti-tumor activity.

Nucleic acid vaccines pose a paradoxical problem. The delivery of exogenous, synthetic DNA or RNA to cells triggers innate immune responses (for example, by STING itself in response to DNA, as described above), particularly IFN Type I. While this has often been historically cited as beneficial, Type I IFN signaling in the context of RNA vaccines leads directly to the phosphorylation (and thus deactivation) of eukaryotic translation initiation factor 2α (eIF2α) to globally repress translation, and there is evidence that this and other mechanisms effectively shut down translation of exogenous RNA (Tesfay et al., Journal of Virology, 2008, vol. 82 (6), pp. 2620-2630, which is incorporated herein by reference as if fully set forth). Thus, a nucleic acid vaccine that is a potent Type I IFN trigger (either due to the nucleic acid itself acting as a pathogen-associated molecular pattern [PAMP] or deliberate inclusion of synthetic STING ligands as adjuvants) may be inherently self-limiting in some applications.

An appealing approach for the enhancement of nucleic acid vaccines would be a method by which the STING pathway is specifically activated at the time of maximal antigen steady-state concentration, and not exclusively at the time of administration of the vaccine. This ensures that the innate immune response occurs in concert with presentation of the encoded antigen to the adaptive immune system, directing adaptive biological mechanisms against the desired target as opposed to allowing IFN-mediated shutdown of the target antigen's production. This approach may be realized by delivery of nucleic acid molecules that directly mediate STING pathway activity by encoding protein products with the desired STING-associated activity, with expression calibrated by design and/or copy-number dose to lead to optimal signaling upon antigen accumulation.

Genetically-controlled STING signaling could benefit any type of nucleic acid vaccine for the reasons stated above. However, particular benefit may be granted in the context of RNA vaccines. As STING is essentially a cytoplasmic DNA sensor, it is independent of the pathways that would be stimulated by RNA sensing innate systems (e.g., PRK, TLR7, etc.). As a parallel but independent pathway therefore, stimulation of this signaling system could be hypothesized to be synergetic with an RNA vaccine, and not subject to inhibitory feedback mechanisms that may dampen its activity in the context of DNA vaccines.

Genetically-delivered STING has a further distinguishing property compared to conventional small-molecule adjuvants: small molecule agonists are not likely to act only at the cellular site of RNA nanop article uptake as they are diffusive and may trigger responses over a greater area. With optimal nucleic acid delivery, one can guarantee the antigen and the encoded STING components are expressed by the same cells, focusing the site of adjuvanting activity to be more restricted to cells taking up the RNA payloads.

A genetic means of triggering a STING-mediated Type I IFN response for use in the context of nucleotide vaccination is described here. By encoding constitutively active STING in the RNA payload of a vaccine, the activation of the TBK1-IRF3 signaling axis is directed at a time post-entry of the RNA that would be approximately concurrent with antigen accumulation.

The immunostimulatory action of most nucleic acid-based vaccines is believed to be due to the detection of exogenous synthetic DNA or RNA in the cytoplasm by an array of innate immune detector proteins. Regardless of specific mechanism, the induction of the Type I IFN response crucial to the immunogenicity of RNA vaccines must be counterbalanced by the need to ensure sufficient antigen translation from the RNA, which the IFN response eventually suppresses. The optimal means of inducing the IFN response to RNA vaccines is therefore an essential element of design, potentially sensitive to the nature of the specific RNA payload, means of RNA delivery, target cell, etc.

The STING-based adjuvanting of nucleic acid vaccines described herein is based on expression of STING at the same site of antigen delivery. STING-mediated activation of Type I IFN response using this approach generates potent innate immune responses that coincide with accumulation of antigen, improving immunogenicity while simultaneously circumventing early translational repression of the exogenous nucleic acid molecule. The delivered nucleic acid can be a single or multiple molecules of DNA or RNA, modified or unmodified, in combination or hybrid forms. Rate of translation and steady-state concentrations of antigen and STING proteins can be controlled by various methods (selection of copy number in the final formulation, encoding stability determinants, use of different promoters/regulatory elements, etc.).

Studies have shown that STING activation results in expression of factors that promote IFN Type I (alpha/beta) expression downstream. Therefore, expression of STING via mRNA transfection is expected to result in IFN Type I expression.

The first step was to demonstrate expression of different human STING molecules in vitro following transfection of cell lines with mRNA encoding the molecule using a commercial transfection reagent. The next step was to show that transfection of cell lines with this RNA results in an increase in IFN stimulated gene expression using IFN reporter cells.

Towards this goal, gene fragments encoding the STING molecules of interest were designed in silico and ordered from Thermo Fisher Scientific, with appropriate 3′ and 5′ flanking sequences to allow for easy cloning into our linearized mammalian expression plasmids using the Takara In-Fusion cloning kit (Cat #638933) according to the manufacturer's instructions. Two designed amino acid sequences carried two different single amino acid point mutations to make the STING protein constitutively active, a third sequence was designed to be a double-mutant combining these two point mutations, and the fourth sequence designed was wild-type (WT) STING protein. Namely, they were: STING N154S, STING R284M, STING N154S/R284M, and STING WT.

STING Mutant N154S has the same amino acid sequence as WT STING with a substitution of Asparagine (N) with Serine (S) at the 154th amino acid position. STING Mutant R284M has the same amino acid sequence as WT STING with a substitution of Arginine (R) with Methionine (M) at the 284th amino acid position. STING Double Mutant N154S/R284M has the same amino acid sequence as WT STING with a substitution of Asp aragine (N) with Serine (S) at the 154th amino acid position as well as a substitution of Arginine (R) with Methionine (M) at the 284th amino acid position. To serve as a negative control, a clone of STING N154/R284M carrying a truncating frameshift mutation (STING FS) and thus possessing no activity was also generated.

The amino acids of STING proteins and nucleic acids encoding these proteins are listed in Table 1.

TABLE 1
List of STING amino and nucleic acid sequences

| SEQ ID | | Nucleic Acid (NA)/ |
|---|---|---|
| NO | Description | Amino Acid (AA) |
| 1 | WT STING protein | AA |
| 2 | WT STING coding | DNA |
| | sequence | |
| 9 | WT STING coding | RNA |
| | sequence | |
| 3 | STING N1545 | AA |
| | mutant | |
| 4 | STING N154S | DNA |
| | mutant coding | |
| | sequence | |
| 10 | STING N154S | RNA |
| | mutant coding | |
| | sequence | |
| 5 | STING R284M | AA |
| | mutant | |
| 6 | STING R284M | DNA |
| | mutant coding | |
| | sequence | |
| 11 | STING R284M | RNA |
| | mutant coding | |
| | sequence | |
| 7 | STING | AA |
| | N154S/R284M | |
| | mutant | |
| 8 | STING | DNA |
| | N154S/R284M | |
| | mutant coding | |
| | sequence | |
| 12 | STING | RNA |
| | N154S/R284M | |
| | mutant coding | |
| | sequence | |
| 16 | STING FS protein | AA |
| 17 | STING FS coding | DNA |
| | sequence | |
| 18 | STING FS coding | RNA |
| | sequence | |

Each fragment was resuspended in H₂O and the In-Fusion reaction was carried out according to the manufacturer's instructions.

The DNA plasmids were transformed into and propagated in bacteria according to the manufacturer's instructions (Stellar Competent Cells—Takara cat#636766). Clonal isolates were subjected to plasmid purification using the E.Z.N.A. Miniprep kit I (VWR cat #101318-898) and submitted for sequencing to confirm all sequences and mutations.

RNA was synthesized from the DNA plasmids described above using the MegaScript T7 Transcription Kit from Invitrogen (ThermoFisher Cat#AMB13345), and RNA was capped and tailed using the CellScript ScriptCap™ Cap 1 Capping System (CellScript Cat #C-SCCS1710) and CellScript A-Plus™ Poly(A) Polymerase Tailing Kit (CellScript Cat#PAP60704K) according to manufacturer instructions.

To test the expression and activity of the encoded STING polypeptides in vitro, Murine B16 Blue IFN alpha/beta (Type I) reporter cells and Murine B16 Blue IFN gamma (Type II) reporter cells were transfected in 12-well dishes using the TranslT®-mRNA Transfection Kit from Mirus (Mirus cat#MIR 2250) according to the manufacturer's instructions.

Forty eight hours post-transfection, 20 μL of conditioned media from each culture was analyzed using QuantiBlue assay (InvivoGen). 180μL QuantiBlue reagent was combined with 20 μL of media taken directly from each sample well. Readings were taken after 1 hr by measuring absorbance in the 620-655nm range as directed by the manufacturer's protocol. For Negative Control, 20 μL of unconditioned media was added to 180 μL QuantiBlue Reagent. STING FS was included in these experiments as an additional negative control because it is of a similar length and sequence as the other STING clones, differing only in the frame shift described herein that results in elimination of full-length protein production. The “No Transfection” samples are samples where media taken from wells with cells that had not been transfected. These samples were treated the same way as all other samples in all steps of the assay.

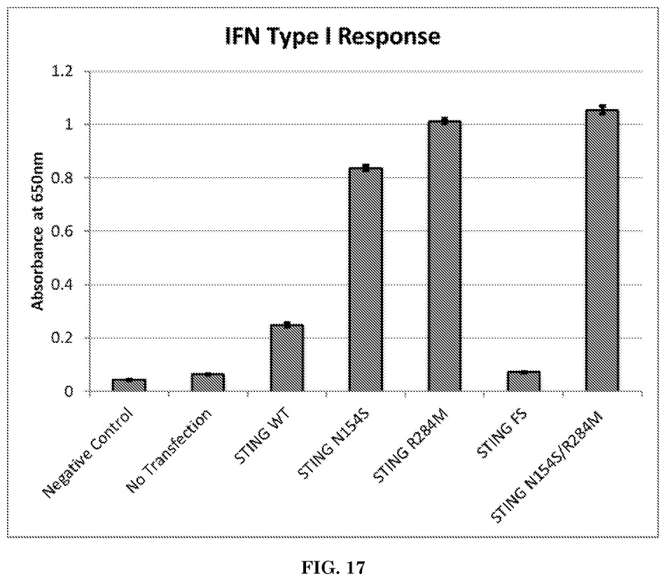

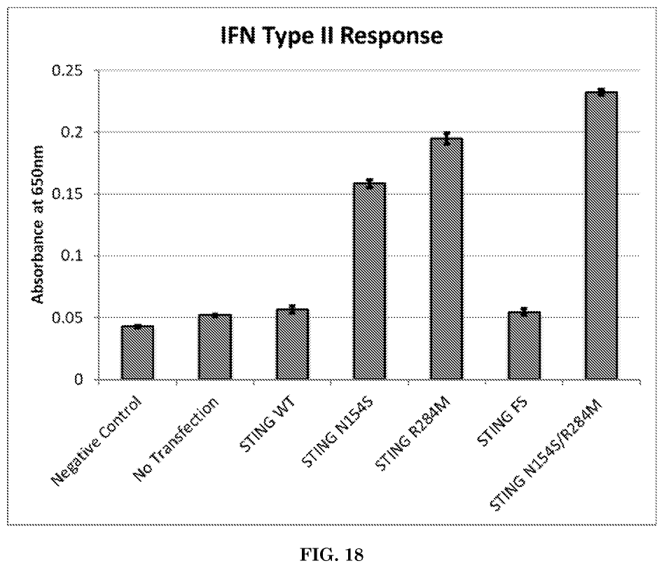

FIG. 17 shows the IFN Type I responses to nucleic acids encoding N154S, R284M, N154S/R284M, and FS STING proteins in reporter cells compared to wild type (WT) construct. The highest response was observed for the N154S/R284M construct. FIG. 18 shows the IFN II responses to nucleic acids encoding N154S, R284M, N154S/R284M, and FS STING proteins compared to WT. The highest response was also observed for the N154S/R284M construct.

After the QuantiBlue assay, cell lysates were harvested from all wells for a Western blot. Cell monolayers were scraped and resuspended in 80 μL of lysis buffer (20 mM Tris-HCl, pH 7.5, 150 mM NaCl, 1 mM EDTA, 1 mM EGTA, 1% NP-40, 1% sodium deoxycholate, 2.5 mM sodium pyrophosphate, 1 mM β-glycerophosphate, supplemented with HALT protease inhibitor to lx final concentration [Thermo Fisher Scientific Inc.]), and left at room temperature (RT) for 5 minutes to lyse. Sixteen microliters of 6× sodium dodecyl sulfate (SDS) Loading Dye (Thermo Fisher Scientific Inc.) was added, and samples were heated for 15 minutes at 95° C. Twenty five microliters of each sample was separated on 4-12% gradient SDS-PAGE gels (Invitrogen), and transferred to PVDF membranes using the iBlot 2 Transfer system (Invitrogen). Membranes were blocked in tris buffered saline+0.5% TWEEN 20 (TBST)+5% milk and proved with primary rabbit monoclonal antibody against STING (D2P2F from Cell Signaling) to reveal protein bands at ˜34 kDa.

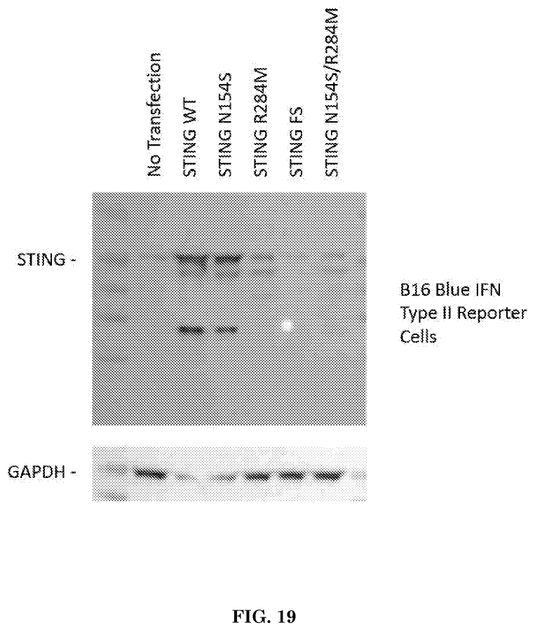

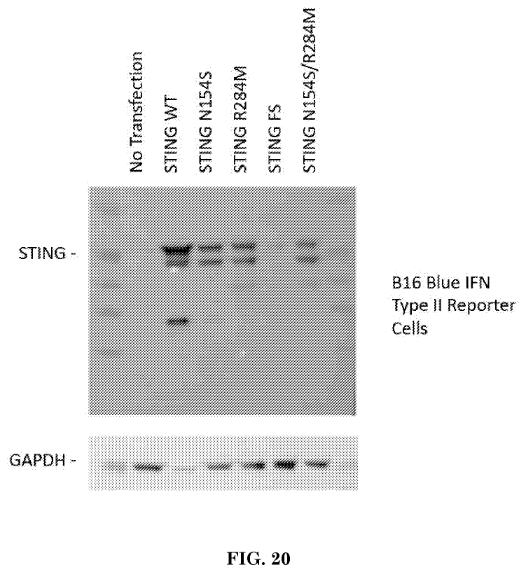

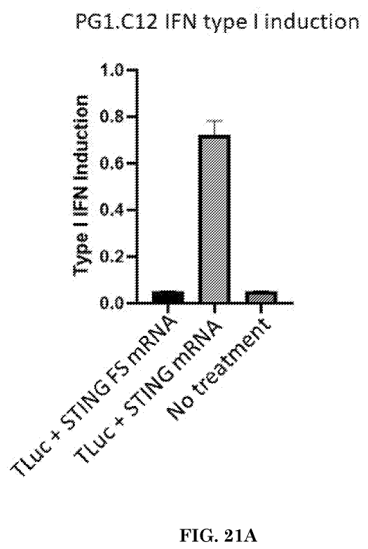

FIG. 19 shows by Western blot the expression level of STING WT, N154S, R284M, FS and N154S/R284M, proteins the IFN Type I Reporter Cells. FIG. 20 shows by Western blot the expression level of STING WT, N154S, R284M, FS and N154S/R284M proteins in the IFN Type II Reporter Cells.

It was observed that STING activation using mRNA encoding it resulted in expression of factors that promote IFN Type I responses downstream. Therefore, expression of STING via mRNA transfection results in IFN Type I-dependent reporter expression. Surprisingly, substantial IFN Type II expression was observed in this experiment. To test this hypothesis, the Type II B16 Blue IFN-gamma reporter cell line was included in the described experiments. The cells were also analyzed for the presence of the encoded STING protein. Referring to FIGS. 19 and 20, clear increases in expression of STING were observed in cells transfected with WT, N154S, R284M, and N154S/R24M. Expression of the double mutant was lower than that of the single mutants but higher than that of the non-transfected samples. An increase in expression was not observed in cells transfected with STING FS because it contains a frameshift mutation that results in a different series of amino acids which is not bound by the STING antibody used in the Western blots. In the Type I IFN reporter assay, an increase in Type I-dependent reporter gene activity was observed, which correlates with the Western blot data. Both single mutants and the double mutant showed greater reporter expression than WT, indicating that these mutations result in increased STING-mediated activation. Surprisingly, an increase in the expression of IFN Type II promoters was observed only upon expression of STING mutants N154S, R284M, and N154S/R284M, although the increase was on a smaller scale than that of IFN Type I. This may be due to crosstalk between the IFN Type I and IFN Type II activation pathways. STING has been reported to activate the transcription factor NF-kB, which could explain the cross-over activation of the IFN-gamma-related (e.g., the Type-II sensitive) promoters as a result of the highly constitutive activity of the selected STING mutants (Abe and Barber, 2014, Journal of Virology, 88(10): 5328-5341). This is a surprising result, but highly fortuitous as activation of both Type I and II branches of innate IFN signaling is highly beneficial in general for the enhancement of vaccine potency.

### Example 9. Incorporation in a Nanoparticle Formulation of an mRNA that Encodes an Active STING Protein, in Addition to Another mRNA that Encodes Another Protein (the Desired Gene/Antigen)

The designs of RNA contents for nanoparticles containing antigen and STING RNA sequences:

Antigen-STING fusions: The antigen and STING may be connected (either in the order antigen-STING, or STING-antigen) at the protein level by direct fusion of the two open reading frames (ORFs), or by a sequence of amino acids that: (i) constitute a flexible, water-soluble, and disordered in nature (e.g., glycine/serine linker); (ii) is cleavable, either by self-cleaving activity or by sequence recognition for a protease provided in trans or endogenous in the target cell; and/or (iii) constitute an ordered domain generating a discrete intact secondary/tertiary structure to confer a specific subcellular localization (e.g., a transmembrane domain)

Antigen and STING RNAs Containing Discrete ORFs:

The antigen and STING are encoded with their own discrete ORFs (either in the order antigen-STING or STING-antigen), separated by an Intervening Genetic Space (IGS) that: (i) contains an element driving translation of the second ORF (e.g., IRES or other cap-independent element); and/or (ii) contains a self-cleaving structure to separate the ORFs into independent RNA molecules by direct cleavage or recombination (e.g., a ribozyme)

Separate mRNA Molecules Encoding an Antigen and STING:

The antigen and STING are encoded with their own separate RNA molecules, where one or both RNA molecules may (i) be a conventional mRNA with 5′ and 3′UTRs derived from natural eukaryotic or viral mRNAs (ii) contain stabilizing or destabilizing sequence elements to control cytoplasmic persistence/stability; (iii) be a replicon RNA (for example, based on alphaviral genomes); and/or (iv) include ORFs for other proteins that may be encoded in independent frames or fused to the antigen or STING ORF, such as by one of the methods described above.

As a proof of concept experiment, nanoparticles including a STING-expressing RNA, STING N154S/R284M, and an exemplary mRNA encoding a luminescent reporter gene TLuc (as a substitute for an antigen) were formulated together and used to treat IFN Type I and Type II reporter cells.

The amino acid sequence of TLuc protein of SEQ ID NO: 13, and TLuc coding sequences, i.e., a DNA sequence of SEQ ID NO: 14, and an RNA sequence of SEQ ID NO: 15 were used. The mRNA encoding any disease-associated antigen can be used instead. The STING N145S/R284M mRNA molecule encoding the protein of SEQ ID NO: 7, and coding sequences, i.e., a DNA sequence of SEQ ID NO: 8, and an RNA sequence of SEQ ID NO: 12 were also encapsulated simultaneously.

This formulation would be expected to drive expression of the desired gene from the second mRNA AND expression of active STING protein from the first mRNA. The active STING protein triggers type I IFN responses directly in the cell.

Modified dendrimer-based RNA nanoparticles formulated with an mRNA encoding constitutively active STING protein in combination with another mRNA provide both IFN type I stimulatory activity and simultaneous gene expression in treated cells.

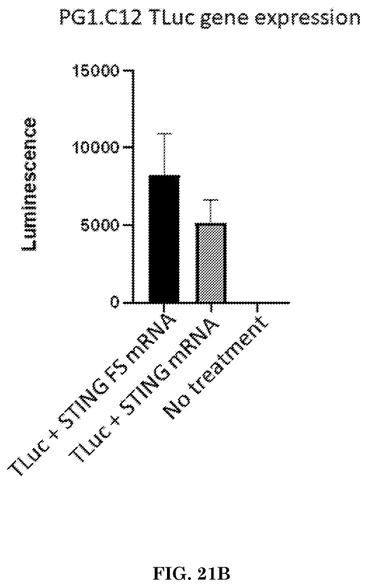

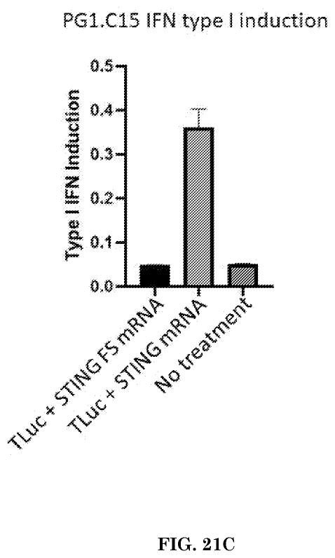

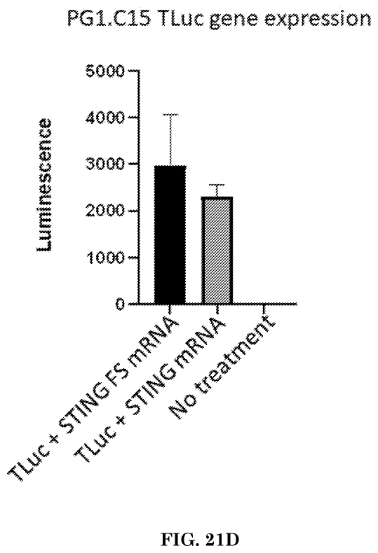

FIGS. 21A-21D illustrate gene expression and activation of the IFN Type I response in the B16 type I reporter cells (B16 Blue IFN I cells, Invivogen) following treatment with the modified-dendrimer (PG1.C12 in FIGS. 21A-21B or PG1.C15 in FIGS. 21C-21D)/PEG-lipid formulated TLuc mRNA in combination with mRNA encoding either STING protein inactivated by a frame-shift mutation (TLuc+STING FS mRNA) or constitutively active STING (double-mutant N154S/R284M; TLuc+STING mRNA).

PG1.C12 Modified Dendrimer-Based RNA Nanoparticles Formulated with both a Model Reporter mRNA (Tluc) and Active STING mRNA Drive Model Gene Expression

FIG. 21A illustrates intensity of IFN type I signaling in B16 type I reporter cells (B16 Blue IFN I cells, Invivogen) treated with the modified-dendrimer PG1.C12 formulations TLuc+STING FS mRNA, TLuc+STING mRNA compared to “No treatment” control. It was observed that only the formulation including active STING mRNA (TLuc+STING mRNA) triggered type I IFN signaling activity.

FIG. 21B illustrates the intensity of TLuc gene expression in B16 type I reporter cells (B16 Blue IFN I cells, Invivogen) treated with the modified-dendrimer PG1.C12 formulations TLuc+STING FS mRNA, TLuc+STING mRNA compared to “No treatment” control. Referring to this figure, TLuc gene expression as quantified by luminescence measurement was observed in both formulations containing either Negative Control inactive STING- or active STING-coding mRNA.

PG1.C15 Modified Dendrimer-Based RNA Nanoparticles Formulated with Both a Model Reporter mRNA (Tluc) and Active STING mRNA Stimulate Type I IFN Signaling

FIG. 21C illustrates intensity of IFN type I signaling in B16 type I reporter cells (B16 Blue IFN I cells, Invivogen) treated with the modified-dendrimer PG1.C15 formulations TLuc+STING FS mRNA, TLuc+STING mRNA compared to “No treatment” control. It was observed that only the formulation including active STING mRNA (TLuc+STING mRNA) triggered type I IFN signaling activity.

FIG. 21D illustrates intensity of TLuc gene expression in B16 type I reporter cells (B16 Blue IFN I cells, Invivogen) treated with the modified-dendrimer PG1.C15 formulations TLuc+STING FS mRNA, TLuc+STING mRNA compared to “No treatment” control. TLuc gene expression as quantified by luminescence measurement was observed in both formulations containing either Negative Control inactive STING- or active STING-coding mRNA.

Production of mRNA TLuc mRNA was produced by cloning TLuc sequence into pcDNA3.1(+) plasmid. This plasmid contains the necessary origin of replication and ampicillin resistance genes necessary for maintenance and propagation in bacterial culture, the mammalian CMV promoter upstream of the gene cloning site to drive expression in mammalian cells in tissue culture, and the bacteriophage T7 transcription promoter downstream of the CMV promoter to allow in vitro transcription of mRNA encoding the cloned genetic sequence that terminates with a BspQI restriction site. STING mRNA was produced by cloning STING constructs into pcDNA3 plasmid cut with XbaI and HindIII. Both constructs were produced using an In-Fusion (Clontech Laboratories) cloning kit. RNAs were generated from BspQI-linearized plasmid vectors by in vitro transcription with T7 MEGAscript kits (Life Technologies) all according to the manufacturer's protocol. All RNA was capped using the Cap1 kit from CellScript, capping 90 pg/reaction and following the manufacturer's protocol. Poly A tail encoded within the plasmid.

Formulation In aqueous solution, a 1:1 STING/TLuc mRNA mixture was prepared by combining equal masses of STING and TLuc mRNA. Nanoparticles were formulated using a dual syringe pump microfluidic mixing device. Briefly, modified dendrimer (either PG1.C12 or PG1.C15) and 1,2-dimyristoyl-sn-glycero-3-phosphoethanolamine-N-[methoxy(polyethyleneglycol)-2000] (PEG-lipid, Avanti Polar Lipids) were combined in ethanol. RNA was diluted with ultraPure, DNase/RNase-free, endotoxin-free distilled water (Invitrogen) and sterile 100 mM (pH 5.0) QB Citrate Buffer (Teknova) to a final citrate concentration of 10 mM, and a final RNA concentration of 0.35 mg/mL. The ethanol and citrate streams were loaded into BD Luer-Lok™ plastic syringes, and using a microfluidic mixing device, the ethanol and citrate streams were combined and mixed in a 1:3 ethanol to citrate stream volumetric flow rate ratio (combined total flow rate equal to 2.8 mL/min) to produce nanoparticles. Nanoparticles were dialyzed against sterile, endotoxin-free PBS using 20,000 molecular weight cut-off Slide-A-Lyzer G2 dialysis cassettes (ThermoFisher). Dialyzed nanoparticles were sterile-filtered using 0.2 μm filters (VWR) and characterized with a Zetasizer NanoZS (Malvern). The concentration of RNA was determined by theoretical mass balance calculations and confirmed by Nano-Drop measurement (Thermo Scientific). The final nanoparticles contained an 11.5:1:2.3 mass ratio of modified dendrimer to PEG-lipid to RNA.

Treatment of Cells Once nanoparticle concentrations were confirmed, B16 Blue IFN Type I SEAP reporter cells (Invivogen) in 96 well tissue culture treated plates (seeded >6 hrs prior to treatment) were treated with 0.3 μg/well, diluted in 50/50 OptiMEM/1×PBS. Cell confluency was 75-85% upon treatment. A total of 100 μL of nanoparticle mixture was applied per well, and cells were left overnight at 37° C. and 5% CO₂.

IFN type I Signaling Activity Assay Approximately 16 hrs post treatment, 20 μL media from each well was placed into a new opaque walled assay plate (Corning 3610). QuantiBlue assay reagent (Invivogen), prepared according to manufacturer's protocol, was warmed to 37° C. and 180 μL was added to each well of media from treated cells. Plate was incubated at 37° C. for about 1 hour until signal could be observed by eye. Plate was read in a Synergy HTX plate reader, using the absorbance setting at 650 nm. TLuc gene expression was quantified in the same dish using the TurboLuc Luciferase One-Step Glow Assay Kit (Thermo Scientific) exactly according to the manufacturer's protocol. Briefly, a volume of assay reagent equal to the volume of medium on the cells was applied to each well and mixed by shaking for 10 min at room temperature, followed by top-of-well measurement of luminescence using a Synergy HTX plate reader.

### Example 10. Incorporation of a Small Molecule Agonist of the Endogenous Cellular STING Protein into RNA Nanoparticle Formulations

The small molecule agonists are called cyclic dinucleotides (CDNs), which describes their chemical structure. Nanoparticle formulations incorporating them will thus not only deliver a desired mRNA (leading to expression of a desired gene/antigen), but trigger type I IFN responses in the cell due to direct binding and activation of STING by the small molecule agonists.

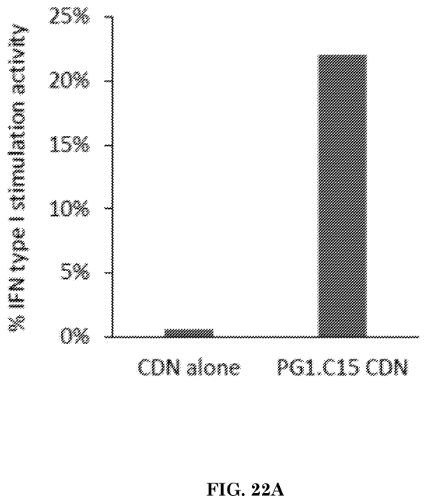

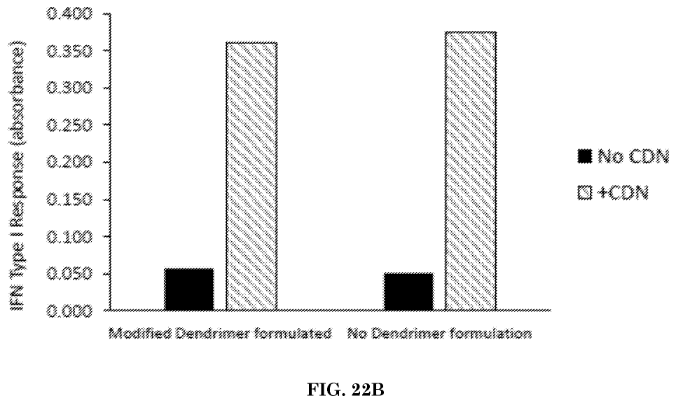

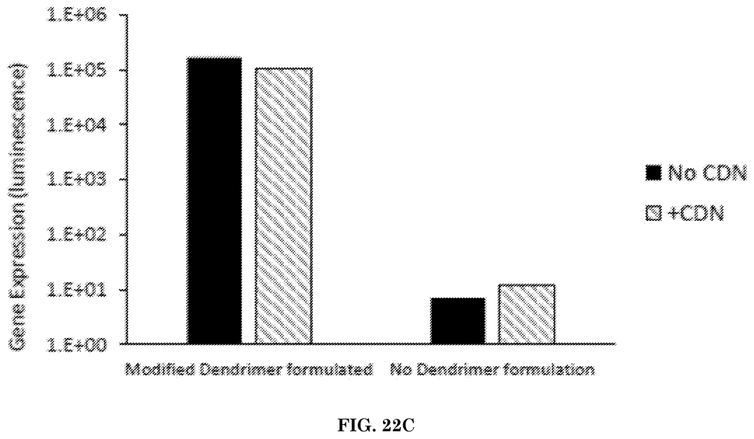

FIGS. 22A-22C illustrate gene expression and activation of the IFN Type I response in the B16 type I reporter cells (B16 Blue IFN I cells, Invivogen) following treatment with the PG1.C15 CDN nanoparticles.

FIG. 22A illustrates intensity of the IFN Type I stimulation activity in the B16 type I reporter cells (B16 Blue IFN I cells, Invivogen) following treatment with the dialyzed modified dendrimer-based RNA nanoparticles (PG1.C15 CDN) formulated with a cyclic dinucleotide (CDN) and normalized to the activity of corresponding pre-dialyzed samples, and CDN alone. It was observed that modified dendrimer-based PG1.C15-nanoparticles PG1.C15 with CDN retain 22% of the IFN type I stimulatory activity on treated cells after buffer exchange to remove free CDN.

The degree of cyclic dinucleotide (CDN) incorporation into mRNA-containing modified dendrimer-based nanoparticles was tested by measuring the level of type I IFN signaling induced by the nanop articles or free CDN alone after overnight dialysis of the formulations against PBS to remove free molecules of molecular weight below 20 000 daltons. The nanop articles contained Tluc mRNA, an example of a functional RNA molecule capable of gene expression.

Production of mRNA TLuc mRNA was produced by cloning TLuc sequence into pcDNA3.1(+) plasmid. This plasmid contains the origin of replication and ampicillin resistance genes necessary for maintenance and propagation in bacterial culture, the mammalian CMV promoter upstream of the gene cloning site to drive expression in mammalian cells in tissue culture, and the bacteriophage T7 transcription promoter downstream of the CMV promoter to allow in vitro transcription of mRNA encoding the cloned genetic sequence that terminates with a BspQI restriction site. The In-Fusion (Clontech Laboratories) cloning kit was used to construct the plasmid from commercially-sourced DNA fragments. RNAs were generated from BspQI-linearized plasmid vectors by in vitro transcription with T7 MEGAscript kits (Life Technologies) all according to the manufacturer's protocol. All RNA was capped using the Cap1 kit from CellScript, capping 90 μg/reaction and following the manufacturer's protocol.

Formulation TLuc mRNA and the CDN 2′3′-c-di-AM(PS)₂(Rp,Rp) (Invivogen) were pre-mixed at a mass ratio of 2:1 before formulation. Nanop articles were formulated by direct mixture of 30 μl of an ethanol phase containing modified dendrimer (PG1.C15) and 1,2-dimyristoyl-sn-glycero-3-phosphoethanolamine-N-[methoxy(polyethyleneglycol)-2000] (PEG-lipid, Avanti Polar Lipids) with 90 μl of RNA/CDN diluted with ultraPure, DNase/RNase-free, endotoxin-free distilled water (Invitrogen) and sterile 100 mM (pH 5.0) QB Citrate Buffer (Teknova) to a final citrate concentration of 10 mM, and a final RNA concentration of 0.35 mg/mL. The ethanol and citrate streams were mixed at a 1:3 ethanol volume to citrate volume ratio to produce nanoparticles. The resulting nanoparticles contained an 11.5:1:2.3 mass ratio of modified dendrimer to PEG-lipid to RNA. For the control “CDN alone” formulation, no dendrimer or PEG-lipid were included in the ethanol phase. Both formulations were dialyzed against sterile, endotoxin-free PBS using 20,000 molecular weight cut-off Slide-A-Lyzer G2 dialysis cassettes (ThermoFisher).

Treatment of Cells B16 Blue IFN Type I SEAP reporter cells (Invivogen) were treated with equal volumes of each formulation ( 1/10^{th} of the final dialyzed volume of each), diluted in 50/50 OptiMEM/1×PBS. Additional wells were similarly treated with samples of the formulation taken before dialysis to measure the level of IFN type I stimulatory activity present in the initial formulation before dialysis to remove free CDN. Cells had been plated in 12 well tissue culture treated plates (WestNetMed) approximately 16 hrs prior to treatment to allow for cells to adhere to the bottom of the well. Cell confluency was 75-85% upon treatment. Cells were treated with a total volume of 600 μL of NP mixture per well, and cells were left overnight at 37° C. and 5% CO₂.

IFN type I Activity Measurement Approximately 16 hrs post treatment, 1 μl of media from each well was placed into a new opaque walled assay plate (Corning). QuantiBlue assay reagent (Invivogen), prepared according to manufacturer's protocol, was warmed to 37° C. and 200 μL was added to each well of media sampled from the treated cells. The plate was put at 37° C. for about 4 hr until colorimetric changes could be clearly observed by eye. The plate was then quantified using a Synergy HTX plate reader, using the absorbance setting at 635 nm. Medium from untreated cells was used to measure the background absorbance value, which was subtracted from all readings. The absorbance reading from dialyzed samples was normalized to the readings from corresponding pre-dialysis samples to reflect nanoparticle retention of CDN activity as a percent of the total amount formulated.

The ability of TLuc mRNA and CDN co-formulated in modified dendrimer nanoparticles to mediate gene expression from the mRNA and simultaneously stimulate type I IFN responses was tested. As a control, a formulation where CDN was excluded was generated and tested in parallel.

FIGS. 22B illustrates intensity of IFN type I signaling in B16 type I reporter cells (B16 Blue IFN I cells, Invivogen) treated with modified-dendrimer/PEG-lipid formulated (PG1.C15; Modified Dendrimer formulated) or unformulated (No Dendrimer formulation) TLuc mRNA and CDN. It was observed that, with and without formulation, the combination of TLuc mRNA and CDN stimulates potent IFN type I activity, whereas TLuc mRNA alone does not.

FIG. 22C illustrates intensity of TLuc gene expression in B16 type I reporter cells (B16 Blue IFN I cells, Invivogen) treated with modified-dendrimer/PEG-lipid formulated (PG1.C15; (No Dendrimer formulation) or unformulated (No Dendrimer) TLuc mRNA and CDN. It was observed that TLuc mRNA was only expressed when included in the formulation. Referring to FIGS. 21B-21C, it was observed that modified dendrimer-based RNA nanoparticles formulated with a cyclic dinucleotide (CDN) provide both IFN type I stimulatory activity and simultaneous gene expression in treated cells.

Production of mRNA TLuc mRNA was produced by cloning TLuc sequence into pcDNA3.1(+) plasmid as described earlier.

Formulation TLuc mRNA and CDN (at a mass ratio of 2:1) or TLuc mRNA alone were prepared as described earlier.

Treatment of Cells B16 Blue IFN Type I SEAP reporter cells (Invivogen) were treated in opaque-walled 96 well dishes with 0.2 μg of each formulation diluted in 50/50 OptiMEM/1×PBS. Cells were treated with a total volume of 100 μL of NP mixture per well, and cells were left overnight at 37° C. and 5% CO₂.

IFN type I Activity and Luciferase Expression Assays Approximately 16 hrs post treatment, 20 μl of media from each well was placed into a new opaque walled assay plate (Corning). QuantiBlue assay reagent (Invivogen), prepared according to manufacturer's protocol, was warmed to 37° C. and 180 μL was added to each well of media sampled from the treated cells. The plate was put at 37° C. for about 30 minutes until colorimetric changes could be clearly observed by eye. The plate was then quantified using a Synergy HTX plate reader, using the absorbance setting at 635 nm. TLuc gene expression was quantified in the same dish using the TurboLuc Luciferase One-Step Glow Assay Kit (Thermo Scientific) exactly according to the manufacturer's protocol. Briefly, a volume of assay reagent equal to the volume of medium on the cells was applied to each well and mixed by shaking for 10 min at room temperature, followed by top-of-well measurement of luminescence using a Synergy HTX plate reader.

### Example 11. Nanoparticles that Include Mixture of Dendrimers with Different Degrees of Self-Assembly Moieties

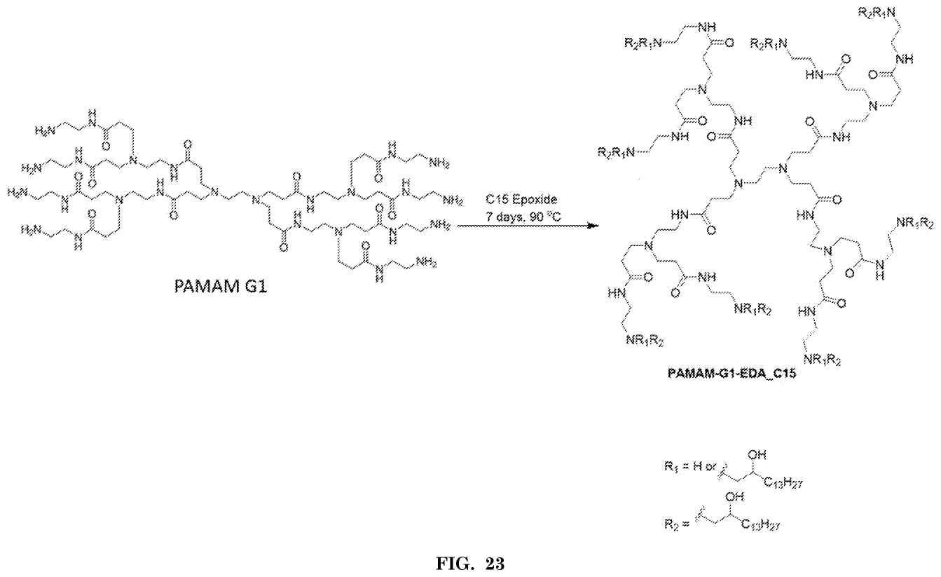

Nanoparticle compositions may include a mixture of modified dendrimers with different amounts of substitution at the terminal layer. For example, the PG1.C15 modified dendrimer was used to make nanoparticles has a terminal layer containing 16 sites available for substitution with an alkyl group. FIG. 23 illustrates the reaction to add alkyl groups to the terminal layer of the PAMAM G1 dendrimer via the primary and secondary amines with the epoxide (2-tridecyclirane (C₁₅H₃₀O), which is described in details in this example. The PAMAM-G1_EDA_C15 (also referred to herein as PG1-C15) is formed, in which R1 is H or C₁₅H₃₁OH, and R2 is C₁₅H₃₁OH. The reaction results in a mixture of modified dendrimers containing GₜZ₁ to GₜZ₁₆, i.e., contain terminal layers having from 1 to 16 alkyl groups resulted from substitutions.

The relative amounts of each molecule in the mixture are unknown and not controlled in the conventional method of preparing modified dendrimers. For this reason, the types of dendrimer molecules in the mixture vary between manufactured batches of the modified dendrimers. Additionally, a lack of control over ratios of dendrimers with different degrees of substitution within the overall dendrimer mixture result in inconsistencies between manufacturing runs. These inconsistencies lead to differences in batch-to-batch performance, or unintended bioactivity. The resulting nanoparticles thus manufactured typically have different performances. For at least these reasons, the conventional method is unfavorable in terms of Chemistry, Manufacturing and Controls (CMC). To improve the molecular definition and reproducibility of the final nanop article product, the ratio between the molecules with different degrees of substitution must be defined. Therefore, a “defined composition” method combining fractions of dendrimers with defined substitution levels is proposed.

Referring to FIG. 23, PAMAM G1 can have can have up to 16 sites capable of substitution in the terminal layer, meaning there can be up to 16 different modified dendrimers produced. In the conventional methods of producing a mixture of modified dendrimers, 2-tridecyloxirane was synthesized by the drop-wise addition of 1-pentadecene (TCI) to a twofold molar excess of 3-chloroperbenzoic acid (Sigma) in dichloromethane (BDH) under constant stirring at room temperature. After reacting for 8 h, the reaction mixture was washed with equal volumes of supersaturated aqueous sodium thiosulfate solution (Sigma) three times. After each wash, the organic layer was collected using a separation funnel. Similarly, the organic layer was then washed three times with 1 M NaOH (Sigma). Anhydrous sodium sulfate was added to the organic phase and stirred overnight to remove any remaining water. The organic layer was concentrated under vacuum to produce a slightly yellow, transparent oily liquid. This liquid was vacuum-distilled (˜6.5 Pa, ˜80° C.) to produce clear, colorless 2-tridecyloxirane. Generation 1 poly(amido amine) dendrimer with an ethylenediamine core (Dendritech) was then reacted with 2-tridecyloxirane. The stoichiometric amount of 2-tridecyloxirane was equal to 1.5-fold the total number of amine reactive sites within the dendrimer (two sites for primary amines and one site for secondary amines). Reactants were combined in cleaned 20-mL amber glass vials. Vials were filled with 200-proof ethanol as the solvent and reacted at 90° C. for 7 days in the dark under constant stirring to ensure the completion of the reaction. The crude product was mounted on a Celite 545 (VWR) precolumn and purified via flash chromatography using a CombiFlash Rf machine with a RediSep Gold Resolution silica column (Teledyne Isco) with gradient elution from 100% CH₂Cl₂ to 75:22:3 CH₂Cl₂/MeOH/NH₄OH_{aq} (by volume) over 40 min. TLC was used to test the eluted fractions for the presence of modified dendrimers using an 87.5:11:1.5 CH₂Cl₂/MeOH/NH₄OH_{aq} (by volume) solvent system. Modified dendrimers with different levels of substitution appeared as a distinct band on the TLC plate. Fractions containing unreacted 2-tridecyloxirane and poly(amido amine) dendrimer were discarded. Remaining fractions were combined, dried under ramping high vacuum for 12 hours, and the mixture was stored under a dry and inert atmosphere until used.

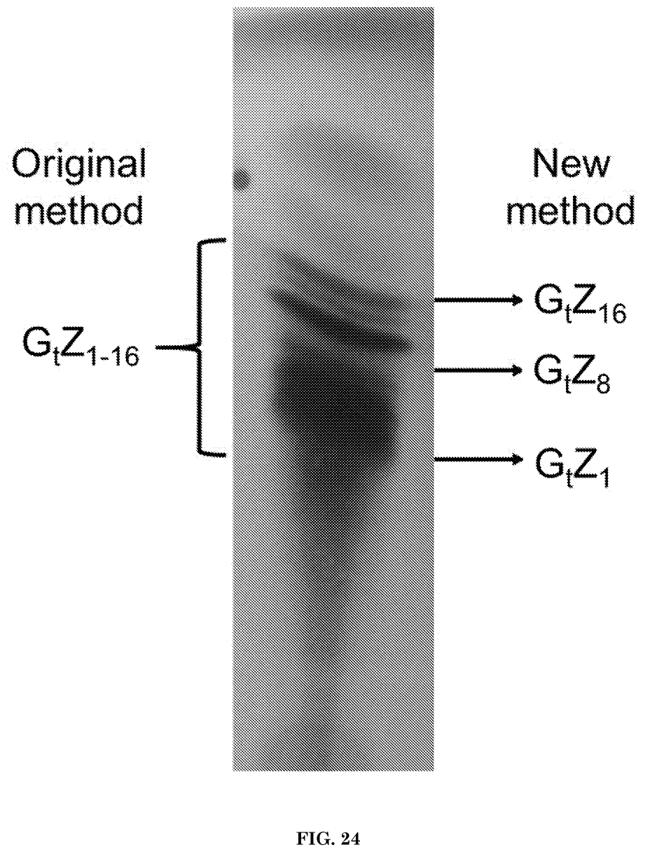

In the “defined” method described herein, 2-tridecyloxirane was synthesized by the drop-wise addition of 1-pentadecene (TCI) to a twofold molar excess of 3-chloroperbenzoic acid (Sigma) in dichloromethane (BDH) under constant stirring at room temperature. After reacting for 8 hours, the reaction mixture was washed with equal volumes of supersaturated aqueous sodium thiosulfate solution (Sigma) three times. After each wash, the organic layer was collected using a separation funnel. Similarly, the organic layer was then washed three times with 1 M NaOH (Sigma). Anhydrous sodium sulfate was added to the organic phase and stirred overnight to remove any remaining water. The organic layer was concentrated under vacuum to produce a slightly yellow, transparent oily liquid. This liquid was vacuum-distilled (˜6.5 Pa, ˜80° C.) to produce clear, colorless 2-tridecyloxirane. Generation 1 poly(amido amine) dendrimer with an ethylenediamine core (Dendritech) was then reacted with 2-tridecyloxirane. The stoichiometric amount of 2-tridecyloxirane was equal to 1.5-fold the total number of amine reactive sites within the dendrimer (two sites for primary amines and one site for secondary amines). Reactants were combined in cleaned 20-mL amber glass vials. Vials were filled with 200-proof ethanol as the solvent and reacted at 90° C. for 7 d in the dark under constant stirring to ensure the completion of the reaction. The crude product was mounted on a Celite 545 (VWR) precolumn and purified via flash chromatography using a CombiFlash Rf machine with a RediSep Gold Resolution silica column (Teledyne Isco) with gradient elution from 100% CH₂Cl₂ to 75:22:3 CH₂Cl₂/MeOH/NH₄OH_{aq} (by volume) over 40 min. TLC was used to test the eluted fractions for the presence of modified dendrimers using an 87.5:11:1.5 CH₂Cl₂/MeOH/NH₄OH_{aq} (by volume) solvent system. Eluted fractions were tested by TLC for their level of substitution. The first eluted fraction contained unreacted 2-tridecyloxirane and was discarded. The next eluted fraction was the highest degree of substitution, followed by eluted fractions with decreasing levels of substitutions. All fractions were separately collected and dried under ramping high vacuum for 12 hours, and stored under a dry and inert atmosphere. When forming nanop articles, the desired amount of each fraction is combined together in the ratio of choice. This defined mixture of modified dendrimers with different substitutions is then mixed with the other necessary components to make the nanoparticles. Thus, the difference between the “defined composition” method described herein and the conventional method lies in the ability of the latter to control the relative ratios and types of dendrimer fractions based on the specific degree of substitution. FIG. 24 illustrates the thin layer chromatography (TLC) plate showing the multiple modified dendrimers produced during a single reaction, each containing a different degree of substitution. Each of these molecules appears as a horizontal band. Modified dendrimers with the highest degree of substitution appear at the top of the chromatogram, and dendrimers with the lowest degree of substitution appear at the bottom. In this chromatogram, many bands are close together and not fully resolved. In the conventional method shown on the left, all modified dendrimers, i.e., GₜZ₁₋₁₆ are collected together, and the mixture was used to produce nanoparticles. In one example of this optimized “defined composition” method, only three distinct levels of terminal layer substitution GₜZ₁ (low level of substitution), GₜZ₈ (intermediate level of substitution) and GₜZ₁₆ (high level of substitution), are collected to prepare the nanoparticles.

Referring to FIG. 24, it was observed that PAMAM G1 can have up to 16 sites capable of substitution, meaning there can be up to 16 different modified dendrimers produced. The benefits of the “defined composition” method include a defined, known ratio of the modified dendrimers in the final nanoparticle product, no batch-to-batch variability, and the prevention of unexpected bioactivity resulting from a random ratio. Additional benefits include controlling steric hindrance and self-assembly during the nanop article formation process. Highly-substituted terminal layers are better for nanop article self-assembly, but there is more steric hindrance that prevents nucleic acids from associating with the amine groups. Lower degrees of substitution have less nucleic acid steric hindrance, but have fewer self-assembly groups. An intermediate level of substitution can link the two aforementioned types together, acting as a bridging molecule. It can associate with the nucleic acids that are partially saturated with the low degree of substitution modified dendrimers, and can self-assemble with the highly-substituted modified dendrimers.

Thus, the method results in better chemistry, manufacturing and control (CMC).

### Example 12. Nanoparticle Compositions Containing Heterogeneous Modified Dendrimers

FIG. 25 illustrates molecular structures of the modified dendrimers containing a core and one, two or three layers. Modified dendrimers are formed by a multi-step synthetic process. Each layer is chemically distinct, as illustrated by the inclusion of different R groups in the core (R₁), in the one-layer dendrimer (R₁ and R₂), in the two-layer dendrimer (R₁, R₂ and R₃), and in the four-layer dendrimer (R₁, R₂, R₃, and R₄). R groups can be selected to alter properties, including the reduction of steric hindrance to promote better nucleic acid association and binding. Modified dendrimers are formed by a multi-step synthetic process. Each layer is chemically distinct, as illustrated by the inclusion of different R groups in the core (R₁), in the one-layer dendrimer (R₁ and R₂), in the two-layer dendrimer (R₁, R₂ and R₃), and in the four-layer dendrimer (R₁, R₂, R₃, and R₄). R groups can be selected to alter properties, including the reduction of steric hindrance to promote better nucleic acid association and binding. The top structure shown on this figure is a core represented by two amines linked through the R₁ functional group. The upper structure in the middle of the figure is a one-layer modified dendrimer, in which hydrogens in the core amines are substituted with additional amine containing moieties also containing R₂ functional groups. The lower structure in the middle of the figure is a two-layer modified dendrimer, in which hydrogens in the core amines are substituted with additional amine containing moieties also containing R₂ or R₃ functional groups. The structure at the bottom of the figure is a three-layer modified dendrimer, in which hydrogens in the amines of the second layer are further substituted with amine containing moieties additionally containing R₄ functional groups. As shown in FIG. 25, additional amines may be added to a layer without adding more layers. The high amine density may be used to prevent nanoparticle recycling after initial endocytosis as it may amplify the proton-pump effect to more quickly rupture the endosomes post-uptake of the nanoparticles comprising the modified dendrimer. The addition of amines to a single layer of the heterogeneous modified dendrimers may be advantageous compared to the homogeneous modified dendrimers containing identical repeating units, wherein another layer must be added for the addition of more amines. The addition of layers to the homogenous modified dendrimers may cause a large increase in molecular weight, and associated with it toxicity, and may hinder clearance. Also, the number of amines added to the homogenous modified dendrimer may be too high. The heterogeneous modified dendrimer may be formed with heterogeneous layers inherently having different number of amines without the need to use repeating units required to add amines for homogeneous molecules. The amine density of the heterogeneous modified dendrimer may be increased by altering a single layer within the molecule without the need to alter or add additional layers. The process of incorporating amines may be controlled in the “heterogeneous” approach. By selecting a layer and functional groups, fewer or more amines may be added to the heterogeneous modified dendrimer. To incorporate fewer amines, a layer closer to the core of the heterogeneous modified dendrimer may be altered. If R₂ is an amine and R₃ is a hydrogen, the modified dendrimer depicted in FIG. 25 (bottom) would incorporate 4 extra amines. To incorporate more amines, a layer farther from the core of the modified dendrimer shown on FIG. 25 (bottom) may be used. If R₂ is a hydrogen and R₃ is an amine, the modified dendrimer shown on this figure would add 8 extra amines. The “heterogeneous” approach for increasing amine density by adding amines to a single layer may overcome the deficiency of homogeneous dendrimers where adding entirely new layers may be required for adding amines. Thus, the “heterogeneous” approach for adding amines may provide more control, at smaller increments and without increasing overall molecular weight compared to the “homogeneous” approach.

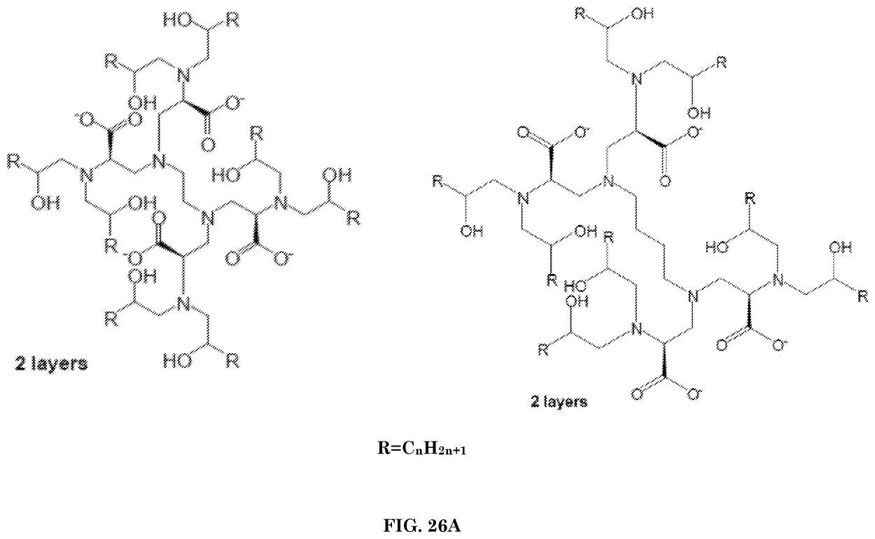

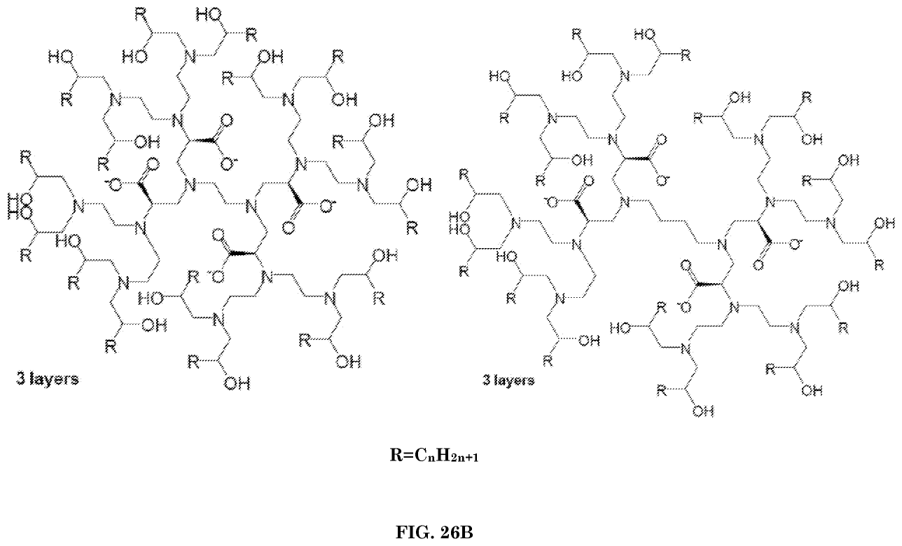

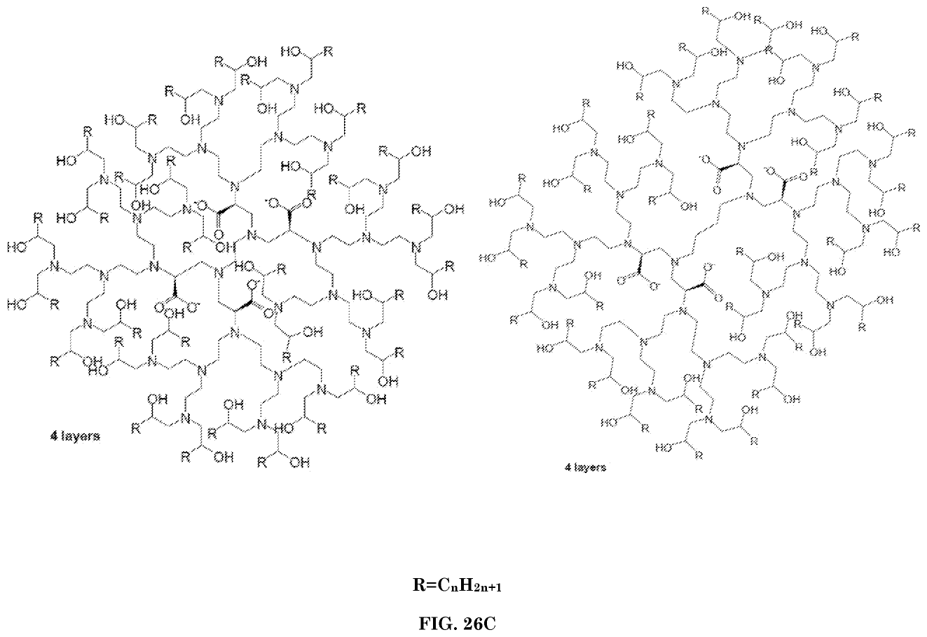

FIGS. 26A-26C illustrate molecular structures of the modified dendrimers containing 1,2 diaminoethane (left) or 1,4 thaminobutane (right) cores, and 2 (FIG. 26A), 3 (FIG. 26B), or 4 (FIG. 26C) layers. In all modified dendrimers shown in this figure, the first layer contains an R group with a carbocyclic acid, which is incorporated to scavenge hydroxide ions. In the depicted structures, R═CₙH₂ₙ₊₁. Additionally, the amines at the terminal layer of all modified dendrimers depicted in this figure are substituted with a carbon chain fully saturated with hydrogens.

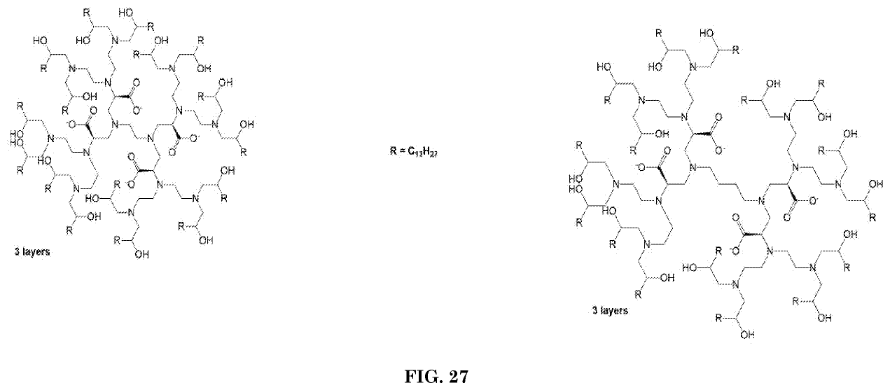

FIG. 27 illustrates molecular structures of three layer modified dendrimers containing a 1,2 diaminoethane core (left structure) and 1,4 thaminobutane as the core (right structure). In the structures depicted in this figure, R═C₁₃H₂₇ The cores can optionally have their carbon or nitrogen atoms replaced with stable isotopes, such as ¹³C or ¹⁵N. The first layer has a carboxylate group, the second layer has a tertiary amine and the third layer a tertiary amine substituted with two pentadecan-2-ol groups.

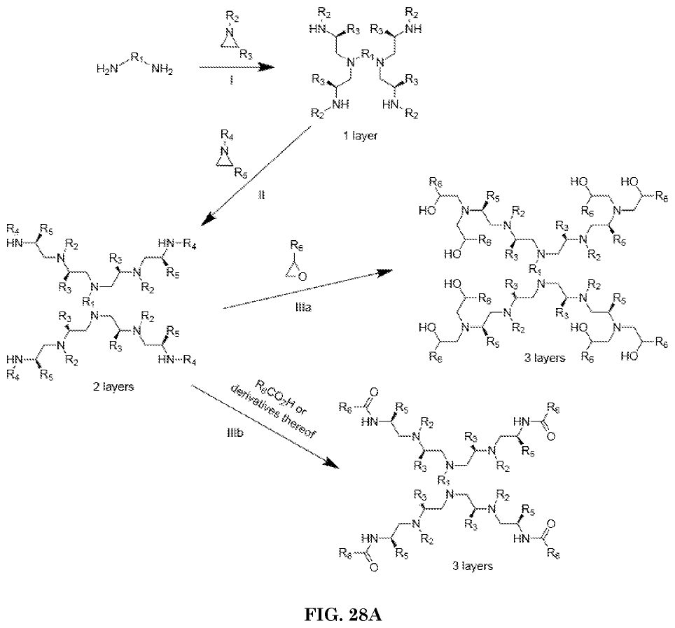

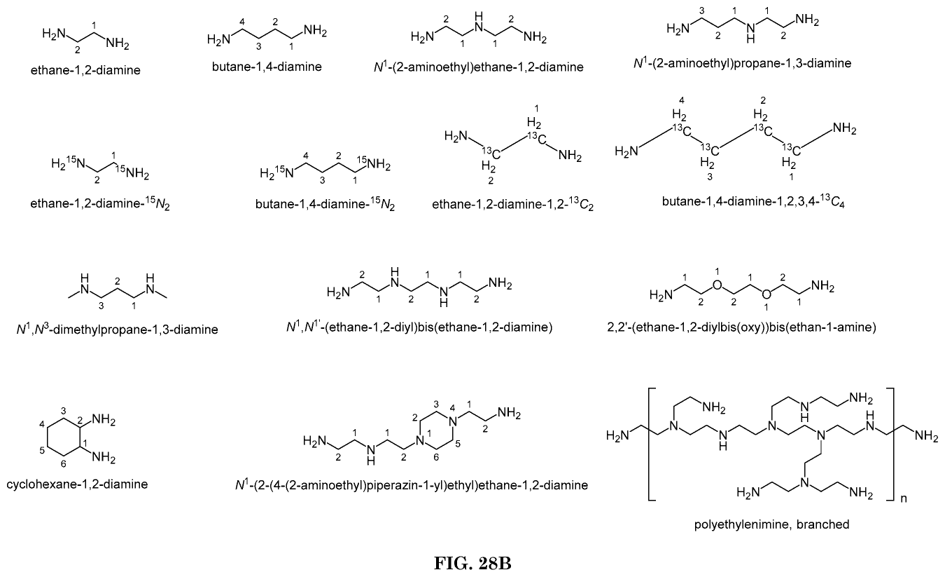

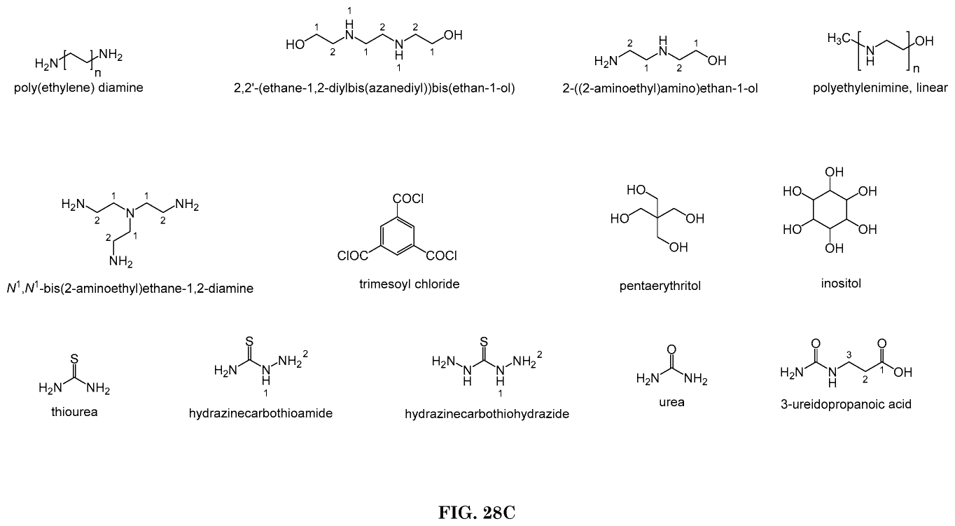

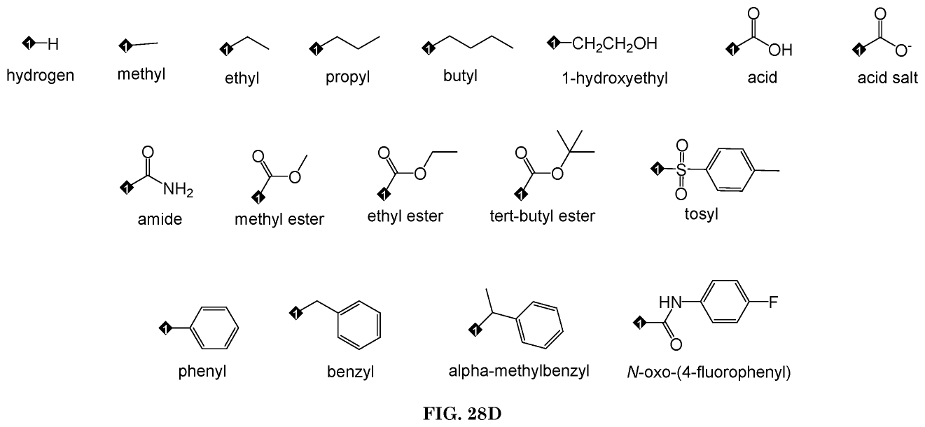

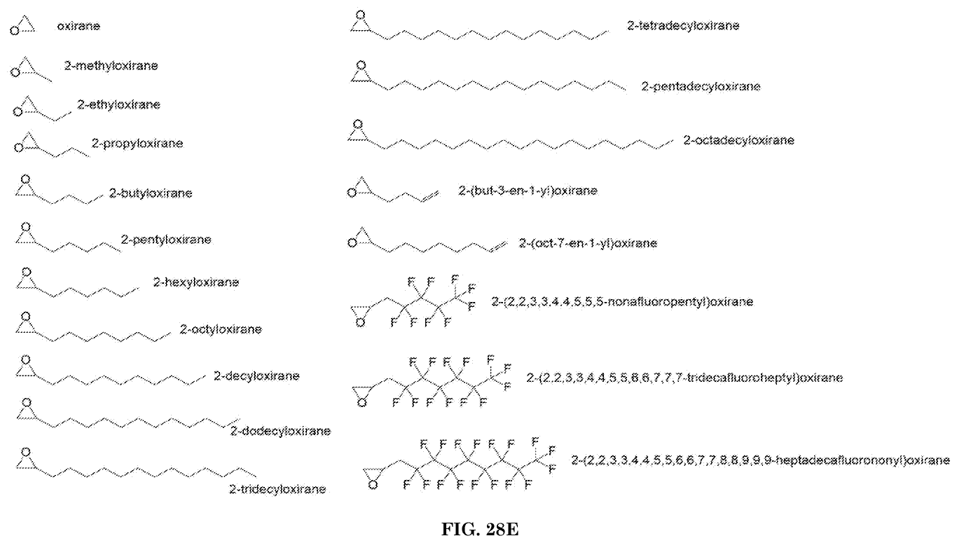

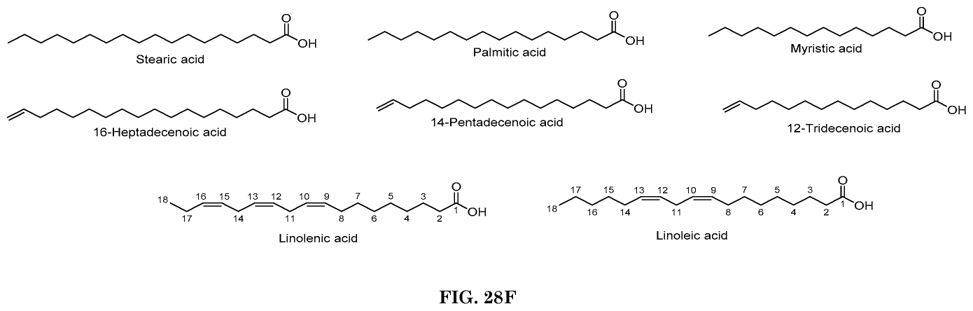

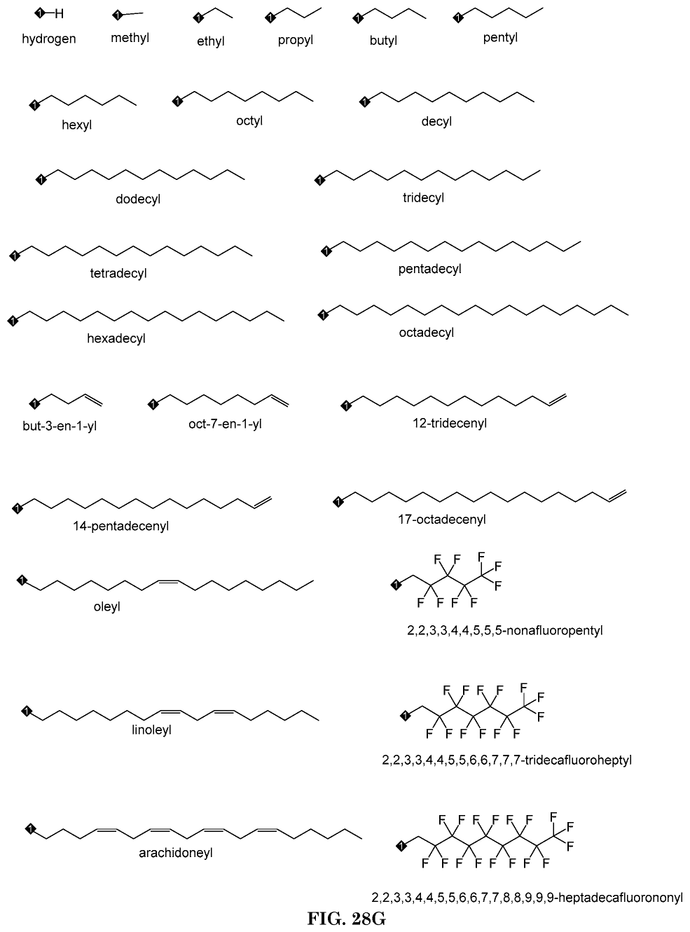

FIGS. 28A-28G illustrate synthesis of the modified dendrimers. FIG. 28A shows steps of general synthesis of a three layer modified dendrimers. Referring to this figure, reaction step I involves the substitution of the primary amines with R2 and R₃ groups via an aziridine reactant to form a 1 layer modified dendrimer. In reaction step II, a 2 layer modified dendrimer is created by substituting the secondary amines of the 1 layer modified dendrimer with R₄ and R₅ groups through a different aziridine reactant. In reaction step IIIa, the secondary amines in the 2 layer modified dendrimer is substituted via Michael addition using an epoxide reactant. In reaction step Mb, the secondary amines in the 2 layer modified dendrimer are reacted with a carboxylic acid (or derivative thereof) in to create an amide bond. FIG. 28B shows molecular structures that can be used as a core of the modified dendrimer: ethane-1,2-diamine; butane-1,4-diamine; N¹-(2-aminoethyl)ethane-1,2 diamine, N¹-(2-aminoethyl)propane-1,3 diamine, ethane-1,2-diamine-¹⁵N₂; butane-1,4-diamine-¹⁵N₂; ethane-1,2-diamine-1,2-¹³C₂; butane-1,4-diamine-1,2,3,4-¹³C₄ N¹, N³-dimethylpropane-1,3-diamine; N¹, N¹-(ethane-1,2-dyl)bis(ethane-1,2-diamine); 2, 2′-(ethane-1,2-dilbys(oxy))bis(ethan-1-amine); cyclohexane-1,2-diamine; N¹-(2-(4-(2-aminoethyl)piperazin-1-yl)ethyl)ethane-1,2-diamine; and polyethylenimine, branched. FIG. 28C shows molecular structures that can be used as a core of the modified dendrimer: poly(ethylene) diamine; 2,2′-(ethane-1,2-dilbys(azanedlyl))bis-(ethan-1-ol); 2-((2-aminoethyl)-amino)ethan-1-ol; polyethylenimine, linear; N¹,N¹-bis(2-aminoethyl)ethane-1,2-amine; trimesoyl chloride; pentaerythritol; inositol; thiourea; hydrazinecarbothiomide; hydrazinecarbothiohydrazide; urea; benzoic acid and 3-ureidopropanoic acid. FIG. 28D illustrates structures of hydrogen, methyl, ethyl, propyl, butyl, phenyl, benzyl, alpha-methylbenzyl, 1-hydroexyethyl, carboxylic acid, carboxylic acid salt, amide, methyl ester, ethyl ester, tertbutyl ester, tosyl, and N-oxo-(4-fluorophenyl) that can be used as R groups for synthesis steps I and II.

FIG. 28E illustrates exemplary reactants used in step III: oxirane, 2-methyloxirane, 2-ethyloxirane, 2-propyloxirane, 2-butyloxirane, 2-pentyloxirane, 2-hexyloxirane, 2-octyloxirane, 2-decyloxirane, 2-dodecyloxirane, 2-tridecyloxirane, 2-tetradecyloxirane, 2-pentadecyloxirane, 2-octadecyloxirane, 2-(but-3-en-1-yl)oxirane, 2-(oct-7-en-yl)oxirane, 2-(2,2,3,3,4,4,5,5,5-nonafluoropentyl)oxirane, 2-(2,2,3,3,4,4,5,5,6,6,7,7,7-tridecafluoroheptyl)oxirane, and 2(2,2,3,3,4,4,5,5,6,6,7,7,8,8,9,9,9-heptadecafluorononyloxirane. FIG. 28F illustrates exemplary reactants used in step III: stearic acid, palmitic acid, myristic acid, 16-heptadecenoic acid, 14-pentadecenoic acid, 12-tridecenoic acid, linolenic acid and linoleic acid. R groups for step III synthesis can be additionally selected from H, C₁-C₁₇ chains (saturated and unsaturated), and fluorinated carbons.

FIG. 28G shows molecular structures of hydrogen, methyl, ethyl, propyl, butyl, pentyl, hexyl, octyl, decyl, dodecyl, tridecyl, tetradecyl, pentadecyl, hexadecyl, octadecyl, but-3-en-1yl, oct-7-en-1-yl, 12-tridecenyl, 14-pentadecynyl, 17-octadecenyl, oleyl, 2,2,3,3,4,4,5,5,5-nonafluoropentyl, linoleyl, 2,2,3,3,4,4,5,5,6,6,7,7,7-tridecafluoroheptyl, arachidoneyl, and 2,2,3,3,4,4,5,5,6,6,7,7,8,8,9,9,9-heptadecafluorononyl that can be used as R groups for step III synthesis.

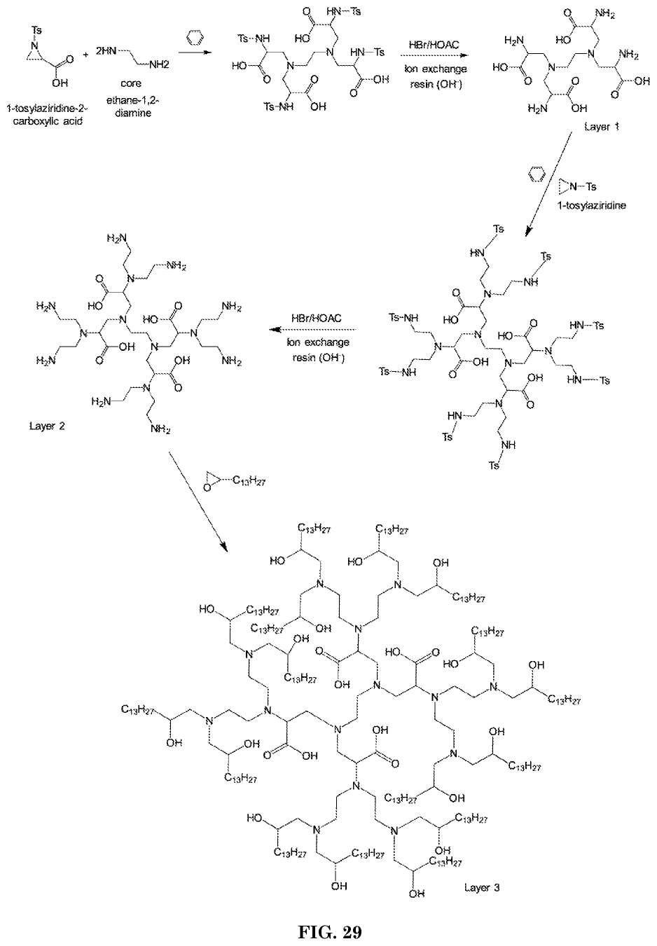

FIG. 29 illustrates synthesis of an exemplary three layer modified dendrimer. Referring to this figure, under argon, 1-tosylaziridine-2-carboxylic acid is dissolved in dry benzene and the solution is cooled in an ice-bath. A solution of freshly distilled core molecule (1,2 diaminoethane) in benzene is added dropwise over 30 minutes while stirring. The mixture is further stirred at room temperature for 24 hours, followed by final stirring at 45° C. (bath temperature) for 72 hours. The solvent is removed under reduced pressure and the colorless oil is purified by column chromatography. The purified material and phenol are combined in a 2:1 stoichiometric ratio and dissolved in a mixture of aqueous HBr (48%) and glacial acetic acid in a 1.7:1 volumetric ratio. The mixture is heated to 130° C. (bath temperature) for 48 hours. The suspension is concentrated by vacuum and residue dissolved in water. After filtration through a short pad of Celite, the solution is concentrated under vacuum to yield a slightly brown solid that contains TsOH as a minor impurity. A column is filled up to a total volume of 200 mL with the ion-exchange resin Dowex 1×8-50 (Cl—) in Millipore water. The material is washed with an excess of Millipore water. The resin was activated with aqueous NaOH (1 weight %, Millipore water). After washing with Millipore water, the column is loaded with crude product in Millipore water. The column is eluted with Millipore water and fractions, that tested basic against litmus paper, are combined and evaporated under reduced pressure (bath temperature less than 50° C.). This produces a 1 layer of the modified dendrimer. This process is repeated to add additional layers. To create a 2 layer modified dendrimer, 1-tosylaziridine is dissolved in dry benzene under argon and the solution cooled in an ice-bath. The 1 layer modified dendrimer in benzene is added dropwise over 30 minutes while stirring. The mixture is further stirred at room temperature for 24 hours, followed by final stirring at 45° C. (bath temperature) for 72 hours. The solvent is removed under reduced pressure and the colorless oil is purified by column chromatography. The purified material and phenol are combined in a 2:1 stoichiometric ratio and dissolved in a mixture of aqueous HBr (48%) and glacial acetic acid in a 1.7:1 volumetric ratio. The mixture is heated to 130° C. (bath temperature) for 48 hours. The suspension is concentrated by vacuum and residue dissolved in water. After filtration through a short pad of Celite, the solution is concentrated under vacuum to yield a slightly brown solid that contains TsOH as a minor impurity. A column is filled up to a total volume of 200 mL with the ion-exchange resin Dowex 1×8-50 (Cl⁻) in Millipore water. The material is washed with an excess of Millipore water. The resin was activated with aqueous NaOH (1 weight %, Millipore water). After washing with Millipore water, the column is loaded with crude product in Millipore water. The column is eluted with Millipore water and fractions, that test basic against litmus paper, are combined and evaporated under reduced pressure (bath temperature less than 50 ° C.). This produces a 2 layer modified dendrimer. The 3 layer modified dendrimer is generated by reacting the 2 layer modified dendrimer with an epoxide molecule. The free amines react with the epoxide via Michael addition reaction at 90° C. in the dark for at least 48 hours while stirring to in 200 proof ethanol produce the crude product. The crude product is mounted on a Celite 545 pre-column and purified via flash chromatography and silica column with gradient elution from 100% CH₂Cl₂ to 75:22:3 CH₂Cl₂/MeOH/NH₄OH_{aq} (by volume) over 40 minutes. Thin layer chromatography (TLC) was used to test the eluted fractions for the presence of a modified dendrimer using an 87.5:11:1.5 CH₂Cl₂/MeOH/NH₄OH_{aq} (by volume) solvent system. Modified dendrimers with different levels of substitution appeared as a distinct band on the TLC plate. Fractions containing unreacted epoxide and modified dendrimers are discarded. Remaining fractions are combined, dried under ramping high vacuum for 12 hours and stored under a dry, inert atmosphere until used.

A major limitation of current nanoparticles is their extracellular recycling. A recent study suggests that less than 2% of endocytosed RNA-laden nanop articles is actually released into the cytoplasm, and other studies have demonstrated that the majority of internalized RNA-laden nanop articles is recycled back to the extracellular environment (Gilleron et al., Nat Biotechnol, 31 (2013), pp. 638-646; and Sahay et al. Nat Biotechnol, 31 (2013), pp. 653-658, both of which are incorporated herein by reference as if fully set forth). The ability to stay and act at the site of administration is critical for delivery efficiency, reproducibility and performance because it prevents wasted payloads, unexpected tropism and off-target delivery, and off-target effects. These limitations are solved by incorporating more amine groups per delivery molecules. More total amines, which include primary, secondary and tertiary amines, will prevent nanoparticle recycling after initial endocytosis through high amine density, which ruptures endosomes post-uptake to stop recycling due to a faster, greatly amplified proton-pump effect. However, a major limitation with incorporating more amine groups per dendrimer is that it increases the generation size and molecular weight, both of which increase toxicity. This limitation is caused by the fact that all layers (generations) of the dendrimer as the same (homogeneous), meaning the only way to incorporate more amines is to incorporate more layers (generations). To solve these limitations, the amine density must be increased without increasing generation size and without a large increase in molecular weight. By making the layers (generations) heterogeneous instead of homogeneous, one can incorporate more amines per layer (generation), resulting in a plurality of layers (generations) where at least one layer has more amines. For example, for the bottom structure labelled “Three layers” in FIG. 25, R₂ can be a hydrogen, H, and R₃ can be an anime, NH₂. For the heterogeneous modified dendrimers with high amine density, the heterogeneous modified dendrimer may contain from 6 to 26 amine groups per molecule. The heterogeneous modified dendrimer with high amine density may also contain 27-58 amine groups per molecule. In some instances, modified dendrimers may contain up to 250 amine groups per molecule.

But, as amines are added to the heterogeneous layers to prevent nanoparticle recycling, the molecular weight increases. Because increases in molecular weight can lead to more toxicity, there is still a need to reduce the molecular weight through a different structural change. In particular, the terminal layer on the modified dendrimer can be altered. The terminal layer of the dendrimer contains amines that are substituted with hydrophobic groups that assist with nanoparticle self-assembly.

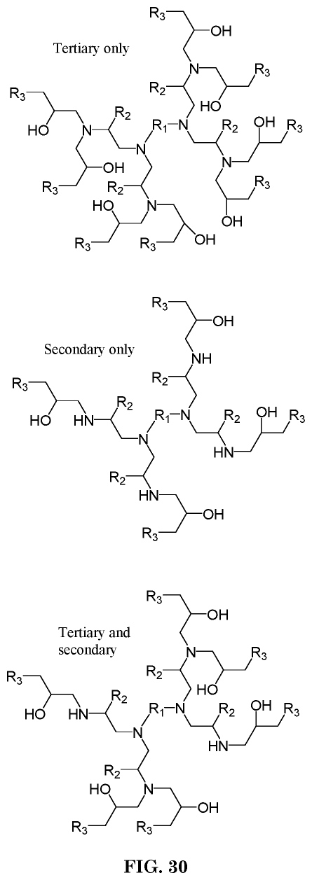

FIG. 30 illustrates modified dendrimers with high level of substitutions (tertiary only; top), low level of substitutions (secondary only; middle), and intermediate level of substitutions (tertiary and secondary; bottom). These heterogeneous multilayer modified dendrimers, each with a different amount of secondary and tertiary amines, can be mixed in a composite that can be further used to form a single nanoparticle. Referring to FIG. 30, the terminal amines in the top structure are typically tertiary amines, which means they are fully substituted with hydrophobic groups. As shown in FIG. 30, middle structure labelled “Secondary only” and lower structure labelled “Tertiary and secondary,” by incorporating more secondary amines in the terminal layer, the terminal amines are no longer fully substituted and thus the overall heterogeneous modified dendrimer will have a lower molecular weight. Another way to reduce the overall molecular weight of the high amine density heterogeneous modified dendrimer is to use shorter hydrophobic groups that have lower molecular weights (the R₄ group in the “Three layers” structure depicted in FIG. 25 and the R₃ group in FIG. 30). Currently, the hydrophobic group is a carbon chain that contains hydrogen-carbon bonds. By replacing the hydrogen-carbon bonds with fluorine-carbon bonds, one can achieve similar hydrophobicity with fewer carbons. As a non-limiting example, this can be done by using one of the fluorine-containing reactants shown in FIG. 28D in the reaction step Ma shown in FIG. 28A. Thus, by replacing the hydrogen-carbon bonds with fluorine-carbon bonds and shortening the length of the carbon chain, the same amount of hydrophobicity is achieved while simultaneously decreasing molecular weight. This decrease in weight offsets the addition of more amines in the heterogeneous modified dendrimer layers. The hydrophobic fluorine-carbon chains may contain between 4 and 11 carbons. The hydrophobic fluorine-carbon chains may also contain between 11 and 20 carbons. Heterogeneous multilayer modified dendrimer can incorporate a layer or multiple layers with many amine groups.

To further prevent nanoparticle recycling, a modified dendrimer can have its terminal layer substituted with unsaturated alkyl groups, which are more fluid (lower crystallization temperature), and thus able to morphologically change into a fusogenic form to help rupture the endosome. The number of unsaturated alkyl groups (R₃ in FIG. 28) in the terminal layer can be increased or decreased to alter the fusogenic capability. In FIG. 28, the most is present in the top structure labelled “Tertiary only,” the least in the middle structure labelled “Secondary only” and an intermediate amount in the bottom structure labelled “Tertiary and secondary.”

Thus, the nanoparticles become restricted and remain inside the cells at the site of injection only and are not trafficked elsewhere. Here, the term “Alkyl” refers to unsaturated aliphatic groups, including straight-chain alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups, or alkyl groups containing alkyl, alkenyl, or alkynyl branches. The alkyl groups can also be substituted with one or more groups including, but not limited to, halogen, hydroxy, amino, thio, ether, ester, carboxy, oxo, and aldehyde groups.

To improve nanoparticle colloidal stability both in solution and self-assembly characteristics, modified dendrimers with their terminal amine layer having different numbers of secondary and tertiary amines are used. For example, nanoparticles can be produced using three kinds of modified dendrimers: (1) modified dendrimers with more secondary amines in their outermost layer, which reduces steric hindrance, allowing more nucleic acid to electrostatically attached to the delivery molecule; (2) modified dendrimers where the outermost layer has tertiary amines, which has greater steric hindrance. This does not electrostatically attach as much nucleic acid payload, but it can promote more nanoparticle self-assembly of nanop articles due to the substituted tails; and (3) modified dendrimers with a mix of secondary and tertiary amines in their final layers. This not only electrostatically attaches nucleic acid, but it acts as a bridge between the two other types of modified dendrimers, thus allowing components to combine into a single nanoparticle.

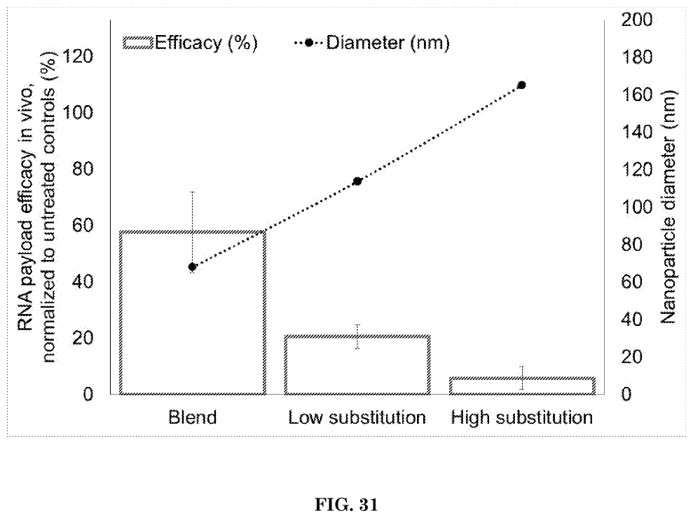

To improve nanoparticle colloidal stability both in solution and self-assembly characteristics, a similar process is also applied to the use of modified dendrimers, where substitution means the amount of, for example, alkylation. FIG. 30 illustrates modified dendrimers with high level (top structure) of substitutions, low level (middle structure) of substitutions and intermediate (bottom structure; tertiary and secondary amine substitutions) level of substitutions. In the structure with high level of substitutions, all H in the amines are substituted (100%) resulting in tertiary amines. In the structure with low level of substitutions, only one H in the amines is substituted (approximately 50%), resulting in secondary amines. In the structure with intermediate level of substitutions, approximately 75% of the available H in the amines are substituted, resulting in a mix of secondary and tertiary amines. Three kinds of modified dendrimers can be mixed to form nanop articles with altered stability in solution and inside the cell (e.g., less stable in cell means faster nucleic acid payload release inside the cell). Three types of modified dendrimers are used: (1) Lower substitution dendrimers, which reduces steric hindrance, allowing more replicon mRNA to be electrostatically attached to the delivery molecule; (2) Highly substituted dendrimers with greater steric hindrance. This does not electrostatically attach as much replicon mRNA payload, but it can promote more nanop article self-assembly of nanop articles due to the substituted tails; and (3) Intermediate substituted dendrimers. This not only electrostatically attaches replicon mRNA, but it acts as a bridge between the high and low substituted versions of the delivery molecule, thus allowing components to combine into a single nanoparticle. FIG. 31 illustrates RNA payload efficacy in vivo for high substitution nanoparticle, low substitution nanoparticle and blend of low, intermediate and high substitution nanoparticles, and correlation of the efficacy with diameters of the nanoparticles. This figure describes the effects of substitution in mice. It was observed that highly substituted modified dendrimers appear larger in diameter and have higher steric hindrance in terms of RNA binding. Low substation modified dendrimer appear intermediate in their diameter and have less steric hindrance in terms of RNA binding. The blend, containing high, low and intermediate levels of substitution, appears smaller in diameter, and is expected to have a more balanced amount of steric hindrance. The intermediate level of substitution helps bind the low and high substitution modified dendrimers into better nanoparticles, which are more efficiently packed (smaller diameter). Nanop articles formed with the mix of different levels of substitution are also more efficacious in mouse animal studies.

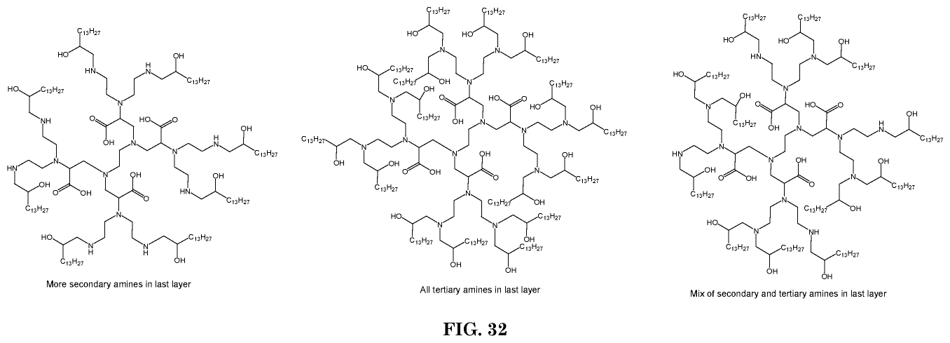

FIG. 32 illustrates exemplary modified dendrimers incorporating secondary and tertiary amines in their terminal (last) layers. In the process of preparing a synthetic vaccine as shown in FIG. 6, the dendrimer may be modified for hydroxide ion-scavenging as described in Examples herein. Referring to FIG. 32, it is shown that hydroxide ion-scavenging modified dendrimers can also have different levels of substitution on their terminal layer.

### Example 13. Ability of Modified Dendrimers and RNA to Form Nnanoparticles

Unmodified PAMAM, PG1.C15 and PG1.C12 were tested for their ability to form nanop articles by direct mixture with Secreted Embryonic Alkaline Phosphatase (SEAP) mRNA. The resulting nanop articles were diluted in PBS and Polydispersity Index (PDI), Y-Intercept (as a measure of signal-to-noise quality of the measurement), Z-average-based diameter, and the derived photon count rate were measured by DLS. Only modified dendrimers were capable of self-assembling with the RNA to generate nanoparticles.

Production of mRNA SEAP mRNA was produced by cloning the SEAP sequence into pcDNA3 plasmid. This plasmid contains the necessary origin of replication and ampicillin resistance genes necessary for maintenance and propagation in bacterial culture, the mammalian CMV promoter upstream of the gene cloning site to drive expression in mammalian cells in tissue culture, and the bacteriophage T7 transcription promoter downstream of the CMV promoter to allow in vitro transcription of mRNA encoding the cloned genetic sequence that terminates with a BspQI restriction site. The In-Fusion (Clontech Laboratories) cloning kit was used to construct the plasmid from commercially-sourced DNA fragments. RNAs were generated from BspQI-linearized plasmid vectors by in vitro transcription with T7 MEGAscript kits (Life Technologies) all according to the manufacturer's protocol. All RNA was capped using the Cap1 kit from CellScript, capping 90ug/reaction and following the manufacturer's protocol.

Formulation Nanoparticles were formulated by direct mixture of 30 μl of an ethanol phase containing modified dendrimer (PG1.C15, PG1.C12) or unmodified PAMAM dendrimer in combination with 1,2-dimyristoyl-sn-glycero-3-phosphoethanolamine-N-[methoxy(polyethyleneglycol)-2000] (PEG-lipid, Avanti Polar Lipids) with 90 μl of SEAP mRNA diluted with ultraPure, DNase/RNase-free, endotoxin-free distilled water (Invitrogen) and sterile 100 mM (pH 5.0) QB Citrate Buffer (Teknova) to a final citrate concentration of 10 mM, and a final RNA concentration of 0.35 mg/mL. The ethanol and citrate streams were mixed at a 1:3 ethanol volume to citrate volume ratio to produce nanoparticles. The resulting nanoparticles contained an 11.5:1:2.3 mass ratio of modified dendrimer to PEG-lipid to RNA. For the “No dendrimer” formulation, no dendrimer of any kind was included in the ethanol phase, while all other components of the ethanol and RNA phase were kept the same. Formulations were diluted 1000-fold for analysis of particle size distribution, Z-average, and derived count rate using a Zetasizer Nano ZS (Malvern P analytical).

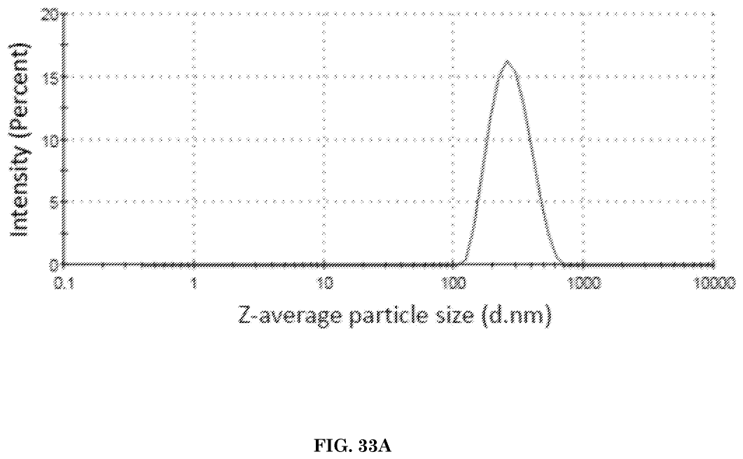

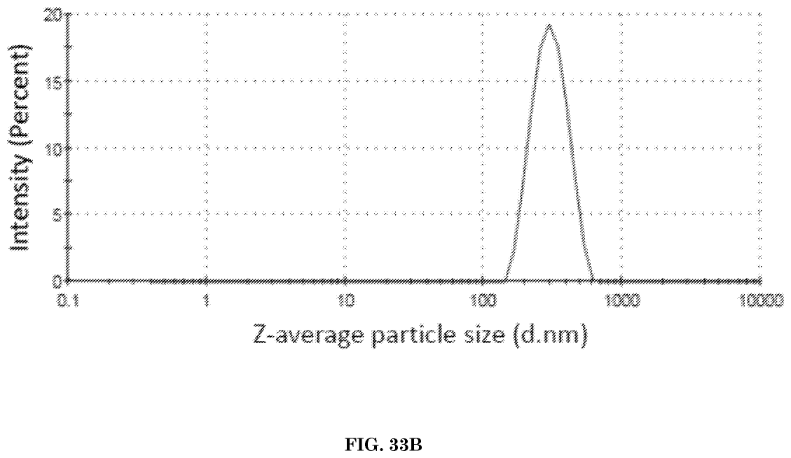

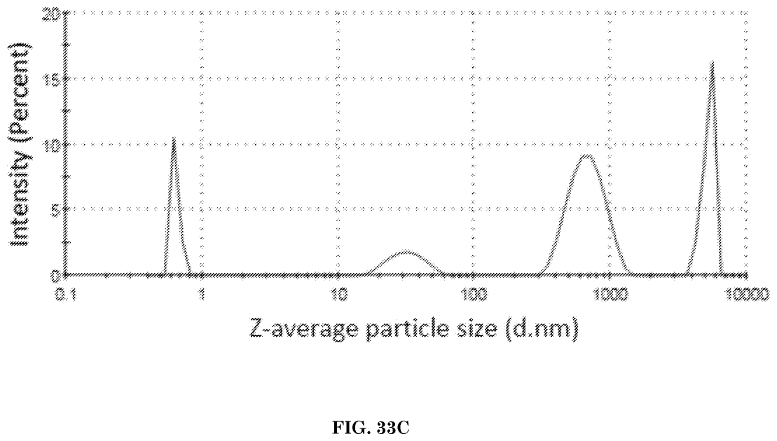

FIG. 33A illustrates particle size distribution of nanoparticles generated by mixture of PG1.C15 modified dendrimer and SEAP mRNA. Referring to this figure, high quality nanoparticles of uniform size were observed.

FIG. 33B illustrates particle size distribution of nanoparticles generated by mixture of PG1.C12 modified dendrimer and SEAP mRNA. Referring to this figure, high quality nanoparticles of uniform size were observed.

FIG. 33C illustrates particle size distribution of the mixture of unmodified PAMAM dendrimer and SEAP mRNA. No consistent or uniform nanoparticles were generated by this material.

Unmodified PAMAM-based formulations with SEAP mRNA do not form nanoparticles as compared to modified dendrimer-based formulations. As shown in the Figure, uniform, consistent nanoparticles are only generated with modified dendrimers are mixed with RNA. No uniform nanoparticle species results from mixture of RNA with unmodified PAMAM. As shown in the Table 2 below, the polydispersity index of the PAMAM mixture is >0.9 indicating high polydispersity (monodisperse suspensions yield PdI of <0.2), the low Y-Intercept value indicates poor signal-to-noise quality (a value of 1 indicates perfect theoretical signal-to-noise detection), and the derived count rate of detected backscattered photons of 7.6 kilocounts per second (kpcs) is even lower than the value detected for a particle-free solution formulated with RNA in the absence of any dendrimer compound (10.6).

TABLE 2
DLS analysis of modified dendrimers PG1.C15 and PG1.C12 or
unmodified dendrimer (PAMAM) mixtures with RNA to evaluate
nanoparticle generation

| | Z-Ave | Polydispersity | | Derived Count |
|---|---|---|---|---|
| Sample Name | (d.nm) | Index (PdI) | Y-Intercept | Rate (kcps) |
| PG1.C15 | 254.1 | 0.113 | 0.955 | 519.2 |
| PG1.C12 | 284.3 | 0.068 | 0.96 | 393.3 |
| Unmodified | 1126 | 0.903 | 0.362 | 7.6 |
| PAMAM | | | | |
| No dendrimer | N/A | N/A | N/A | 10.6 |

Taken altogether, this demonstrates that without modification, PAMAM dendrimers do not self-assemble with RNA to form nanoparticles.FIGS. 33A-33C illustrate particle size distribution of for modified and unmodified dendrimers based on dynamic light scattering (DLS) measurement of nanop articles.

### Example 14. Alkylated Dendrimers Show Improved Nanoparticle Uptake

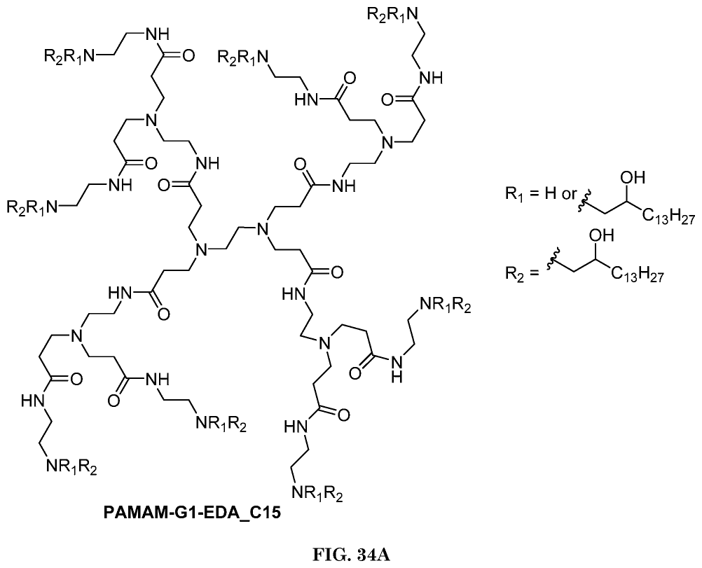

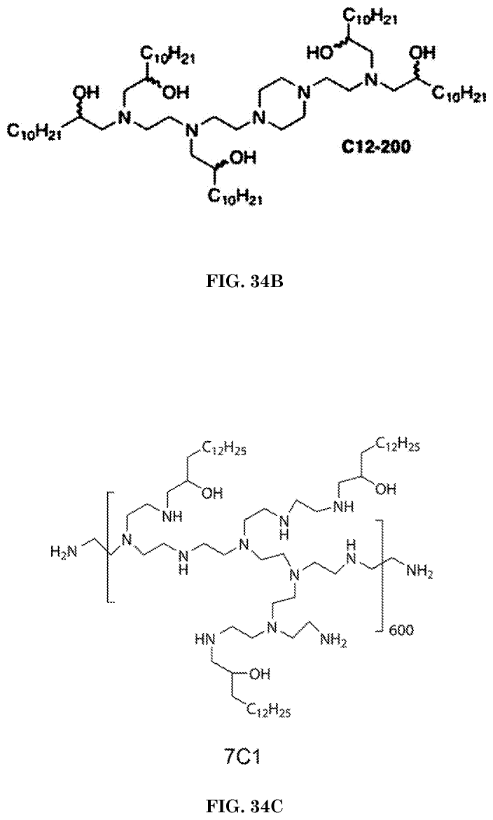

To examine if a modified dendrimer was capable of enhanced cellular uptake, an in vitro system was used. In the system, the performance of the PG1.C15 modified dendrimer was examined. The well-validated polymer 7C1 and the lipomer C12-200 were used to benchmark the performance of the PG1.C15 modified dendrimer. (Dahlman et al. Nat Nanotechnol. 2014; 9(8):648-655; and Love et al., Proc Natl Acad Sci USA. 2010; 107(5):1864-186, which are incorporated herein by reference as if fully set forth). In the PG1.C15 modified dendrimer, “PG1” refers to the core consisting of the first generation poly(amido amine) dendrimer, and “C15” refers to the length of the substituted alkyl chains FIGS. 34A-34C illustrate molecular structures for the PG1.C15 (PAMAM-G1-EDA_C15) modified dendrimer (FIG. 34A), C12-200 lipomer (FIG. 34B) and 7C1 polymer (FIG. 34C).

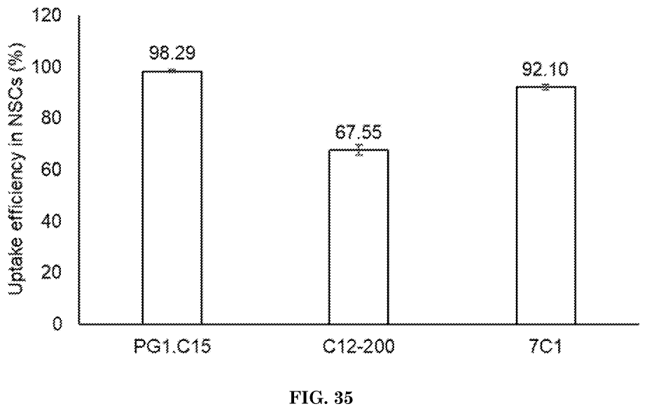

AlexaFluor 647-labelled RNA was formulated into the three types of nanomaterials. Green fluorescent protein-positive (GPF+) human neural stem cells (NSCs) were then treated with 40 nmol (RNA mass) of nanoparticles. FIG. 35 illustrates the uptake efficiency of nanoparticles containing AlexaFluor 647-labelled RNA in human neural stem cells (NSCs) after a 3 hour treatment. RNA dose was 40 nmol. N=12 and error bars are ±S.E.M.

The uptake efficiency (determined by confocal microscopy) was measured as the percentage of GFP+NSCs containing AlexaFluor 647 signal after 3 hours. NSCs treated with the PG1.C15 modified dendrimer showed the highest uptake efficiency of 98.28% in the NSCs. The C12-200 had the lowest uptake of 67.55%, and the lowest amount of ion charge density per molecule.

The 7C1 polymer had more uptake (92.10%) than C12-200, but was still lower than PG1.C15.

### Example 15. Delivery Molecule Molecular Structure Impacts Nanoparticle Recycling

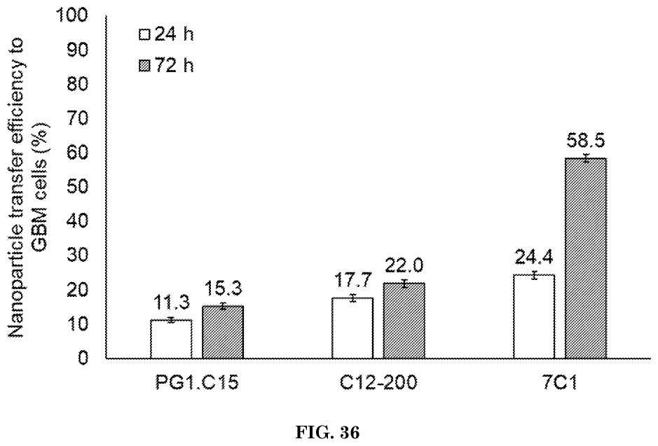

Nanoparticles can be transported in and out of the cell before permanent uptake. This can diminish performance by reducing localized cellular uptake of the nanoparticles, resulting in drainage and premature clearance. To examine if the PG1.C15 modified dendrimer experienced this type of recycling, an in vitro co-culture system was used. Three different classes of nanoparticles, including the PG1.C15 modified dendrimer, the polymer 7C1 and the lipomer C12-200 were used to benchmark the performance of the PG1.C15 modified dendrimer. AlexaFluor 647-labelled RNA was formulated into the three types of nanomaterials. Green fluorescent protein-positive (GPF+) human neural stem cells (NSCs) were then treated with 40 nmol (RNA mass) of nanoparticles. Uptake efficiency (determined by confocal microscopy) was measured as the percentage of GFP+ NSCs containing AlexaFluor 647 signal after 3 hours. To measure nanoparticle recycling, glioblastoma (GBM) cells expressing U87cherry were seeded on top of the nanoparticle-treated GFP+ NSCs. Using confocal microscopy, the percentage of U87cherry+ GBM cells that also showed AlexaFluor 647 signal was quantified; this value was used as a measure of nanoparticles that cycled out of the NSCs and into the adjacent GBM cells. FIG. 36 illustrates the transfer efficiencies of the nanoparticles to glioblastoma (GBM) cells calculated as the percentage of the glioblastoma (GBM) cells containing AlexaFluor 647-labelled nanoparticles that were recycled out of co-cultured human neuronal stem cells (NSCs). White bars are the 24 h time point, and grey bars are 72 h. N=12 and error bars are ±S.E.M.

Referring to FIG. 36, it was observed that GBM cells seeded on top of the PG1.C15 modified dendrimer nanoparticle-treated NSCs showed the least amount of recycled nanoparticle uptake, indicating a lack of PG1.C15 nanoparticle escape post-uptake.

### REFERENCES

Burdette et al., STING is a direct innate immune sensor of cyclic-di-GMP, Nature. 2011 Oct. 27; 478(7370): 515-518.

Chahal and Khan et al., Dendrimer-RNA nanoparticles generate protective immunity against lethal Ebola, H1N1 influenza, and Toxoplasma gondii challenges with a single dose. Proc Natl Acad Sci U S A. 2016 Jul. 19; 113(29):E4133-42.

Chen et al., Using ethidium bromide to probe theinteractions between DNA and dendrimers, Langmuir, 2000, 16:15-19.

Chen, D. et al., Rapid Discovery of Potent siRNA-Containing Lipid Nanoparticles Enabled by Controlled Micro fluidic Formulation, J. Am. Chem. Soc., 2012, 134 (16), pp 6948-6951.

Cong et al., Multiplex genome engineering using CRISPR/Cas systems, Science, 2013 Feb. 15; 339(6121):819-23.

Dahlman J E, et al., In vivo endothelial siRNA delivery using polymeric nanoparticles with low molecular weight, Nat Nanotechnol. 2014; 9(8):648-655

Dubensky, Jr. et al., Rationale, progress and development of vaccines utilizing STING-activating cyclic dinucleotide adjuvants, Ther Adv Vaccines. 2013 November; 1(4): 131-143.

Gao et al., Structure-function analysis of STING activation by c[G(2′,5′)pA(3′,5′)p] and targeting by antiviral DMXAA, Cell., 2013, Aug. 15; 154(4): 748-62.

Gilleron et al., Image-based analysis of lipid nanoparticle-mediated siRNA delivery, intracellular trafficking and endosomal escape, Nat Biotechnol, 31 (2013), pp. 638-646.

Hanson et al., Nanoparticulate STING agonists are potent lymph node-targeted vaccine adjuvants, J Clin Invest. 2015; 125(6):2532-2546.

Ishikawa and Barber, STING an Endoplasmic Reticulum Adaptor that Facilitates Innate Immune Signaling, Nature. 2008 Oct. 2; 455(7213): 674-678.

Janaszewskeet al., Cytotoxicity of Dendrimers, Biomolecules 2019, 9, 330; doi:10.3390/biom9080330.

Jensen et al., Elucidating the molecular mechanism of PAMAM-siRNA dendriplex self-assembly: effect of dendrimer charge density, Int J Pharm. 2011; 416:410-418.

Jinek et al., A programmable dual-RNA-guided DNA endonuclease in adaptive bacterial immunity, Science, 337(6096):816-21 (2012).

Khan et al., Ionizable Amphiphilic Dendrimer-Based Nanomaterials with Alkyl Chain-Substituted Amines for Tunable siRNA Delivery to the Liver Endothelium In Vivo, Angew Chem Int Ed Engl. 2014 Dec. 22; 53(52): 14397-14401.

Khan et al., Dendrimer-Inspired Nanomaterials for the In Vivo Delivery of siRNA to Lung Vasculature, Nano Lett. 2015 May 13; 15(5): 3008-3016.

Love K T, et al., Lipid-like materials for low-dose, in vivo gene silencing, Proc Natl Acad Sci USA. 2010; 107(5):1864-186.

Oliveira S, van Rooy I, Kranenburg O, Storm G, Schiffelers R M, Fusogenic peptides enhance endosomal escape improving siRNA-induced silencing of oncogenes, Int J Pharm, 2007 Mar. 1; 331(2):211-4.

Palmerston Mendes et al., Dendrimers as Nanocarriers for Nucleic Acid and Drug Delivery in Cancer Therapy, Molecules 2017, 22(9), 1401.

Sahay et al., Efficiency of siRNA delivery by lipid nanoparticles is limited by endocytic recycling, Nat Biotechnol, 31 (2013), pp. 653-658.

Takagishi, Y., Sato, K., Tomita, K., and Sakamoto, T., 1979, High-Performance Liquid Chromatographic Determination of Acetohexamide and Its Metabolite, Hydroxyhexamide, Yakugaku Zasshi, 99(9), 961-963. doi:10.1248/yakushi1947.99.9-L961.

Tang, Y., Liu, Y., Xie, Y., Chen, J., Dou, Y., 2019, Apoptosis of A549 cells by small interfering RNA targeting surviving delivery using poly-β-amino ester/guanidinylated O-carboxymethyl chitosan nanoparticles, Asian J. Pharm. Sci., 15:55; doi:10.1016/j.ajps.

Tesfay et al., Alpha/Beta Interferon Inhibits Cap-Dependent Translation of Viral but Not Cellular mRNA by a PKR-Independent Mechanism, Journal of Virology, 2008, vol. 82 (6), pp. 2620-2630.

US20150056224A1, published 2015 Feb. 26.

US20130039933A1, published 2013 Feb. 14.

Van Dyke, R W, Acidification of lysosomes and endosomes, Subcell Biochem. 1996; 27:331-60.

Yan et al., Synthesis and immunostimulatory properties of the phosphorothioate analogues of cdiGMP, Bioorg Med Chem Lett. 2008 Oct. 15; 18(20):5631-4.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A nanoparticle composition comprising a modified dendrimer, a therapeutic or immunogenic nucleic acid agent enclosed within the nanoparticle composition, wherein the modified dendrimer comprises a plurality of terminal amine groups substituted with fatty acids or derivatives thereof.

2. The nanoparticle composition of claim 1, wherein the modified dendrimer comprise a dendrimer selected from the group consisting of: a polyamidoamine (PAMAM) dendrimer, poly(propylene imine) (PPI) dendrimer and poly ethylene imine (PEI) dendrimer.

3. The nanoparticle composition of claim 2, wherein the modified dendrimer is a generation 0, generation 1, or generation 2 dendrimer.

4. The nanoparticle composition of claim 1, wherein the modified dendrimer comprises 100% of the terminal amine groups substituted with fatty acids or derivative thereof.

5. The nanoparticle composition of claim 1, wherein the fatty acids or the derivatives thereof are selected from the group consisting of: arachidonic acid, oleic acid, eicosapentanoic acid, lauric acid, caprylic acid, capric acid, myristic acid, palmitic acid, stearic acid, linoleic acid, and linolenic acid or esters thereof.

6. The nanoparticle composition of claim 1, wherein the modified dendrimer comprises a core selected from the group consisting of: ethylenediamine, diaminobutane, N¹-(2-aminoethyl) ethane, N¹-(2-aminoethyl)propane, N³-dimethylpropan-, N¹,N¹′-(ethane-1,2-diyl)bis(ethane), N¹-(2-(4-(2 aminoethyl)piperazin-1-yl)ethyl)ethane-1,2-diaminecyclohexan, N¹-(2-(4-(2-aminoethyl)piperazin-1-yl)ethyl)ethane-1,2-diaminecyclohexan-, -poly(ethylene)-, -, N¹,N¹-bis(2-aminoethyl)ethane-1,2- diamine, trimesic acid/trimesoyl chloride, pentaerythritol, inositol, thiourea, hydrazinecarbothioamide, hydrazinecarbothiohydrazide, urea, 3-ureidopropanoic acid, ethane-1,2-diamine; ethane-1,2-diamine-¹⁵N₂; ethane-1,2-diamine-1,2-¹³C₂; butane-1,4-diamine; butane-1,2-diamine-¹⁵N₂; butane-1,4-diamine-1,2,3,4-¹³C₂; N¹-(2-aminoethyl)propane-1,3-diamine; N¹-(2-aminoethyl)-N¹-methylethane-1,2-diamine; N¹-methylpropane-1,3-diamine; N¹, N³-dimethylpropane-1,3-diamine; N¹-(2-aminoethyl)ethane-1,2-diamine; and N¹, N³-(ethane-1,2-dyl)bis(ethane-1,2- diamine)thiourea, hydrazinecarbothioamide, hydrazinecarbothiohydrazide, urea, 3-ureidopropanoic acid, 2,2′-(ethane-1,2-diylbis(oxy)bis(ethan-1-amine), 2, 2′-(ethane-1,2-diylbis(azanediyl)bis(ethan-1-ol), 2-((2-aminoethyl)amino)ethan-1-ol; N¹, N¹-bis(2-aminoethyl)ethane-1,2-diamine; N¹-(2-(4-(2-aminoethyl)piperazin-1-yl)ethyl)ethane-1,2-diamine; cyclohexane-1,2-diamine; poly(ethylene)1,n diamine; polyethylenimine, linear and polyethylenimine, branched.

7. The nanoparticle composition of claim 1, wherein the modified dendrimer comprises a tracking moiety.

8. The nanop article composition of claim 7, wherein the tracking moiety is a stable isotope.

9. The nanoparticle composition of claim 8, wherein the stable isotope is a stable isotope of carbon or nitrogen.

10. The nanoparticle composition of claim 9, wherein the stable isotope of carbon is ¹³C, and the stable isotope of nitrogen is ¹⁵N.

11-16. (canceled)

17. The nanop article composition of claim 1, wherein the therapeutic or immunogenic nucleic acid agent is selected from the group consisting of: a polynucleotide, oligonucleotide, DNA, cDNA, RNA, repRNA, siRNA, miRNA, sgRNA, and mRNA.

18. The nanoparticle composition of claim 17, wherein the therapeutic or immunogenic nucleic acid agent encodes one or more antigens selected from the group consisting of infectious disease, pathogen, cancer, autoimmunity disease and allergenic disease.

19. The nanoparticle composition of claim 17, wherein the therapeutic or immunogenic nucleic acid agent comprises an RNA or DNA capable of silencing, inhibiting or modifying the activity of a gene.

20. The nanoparticle composition of claim 17, wherein the nucleic acid agent comprises at least one polynucleotide encoding a STING protein.

21. The nanoparticle composition of claim 20, wherein the STING protein comprises an amino acid sequence with at least 90% identity to a sequence selected from the group consisting of SEQ ID NOS: 1, 3, 5 and 7.

22. The nanop article composition of claim 20, wherein the at least one polynucleotide comprises a sequence with at least 90% identity to a sequence selected from the group consisting of SEQ ID NOS: 2, 4, 6 and 8.

23. The nanoparticle composition of claim 20, wherein the at least one polynucleotide comprises a sequence with at least 90% identity to a sequence selected from the group consisting of SEQ ID NOS: 9-12.

24. The nanoparticle composition of claim 1 further comprising an amphiphilic polymer.

25. The nanoparticle composition of claim 24, wherein the amphiphilic polymer comprises a hydrophilic component selected from the group consisting of: polyalkylene oxides, block copolymers, and polyethylene glycol molecules.

26. The nanoparticle composition of claim 24, wherein the amphiphilic polymer comprises a hydrophobic component selected from the group consisting of: lipid and a phospholipid.

27. The nanoparticle composition of claim 24, wherein the amphiphilic polymer comprises 1,2-dimyristoyl-sn-glycero-3-phosphoethanolamine-N-[methoxy (poly-ethylene glycol)-2000].

28. The nanoparticle composition of claim 1, wherein the nanoparticle composition comprises the amphiphilic polymer in a range from 1% (w/w) to 40% (w/w) of the amphiphilic polymer per nanop article composition.

29-52. (canceled)

53. A method for treating or preventing a disease or condition in a subject comprising:
providing a nanop article composition of claim 1; and
administering a therapeutically effective amount of the nanoparticle composition to a subject.

54. The method of claim 53, wherein the therapeutically effective amount of the nanoparticle composition comprises the therapeutic or immunogenic nucleic acid agent in a range from 0.01 mg nucleic acid to 10 mg nucleic acid per kg body weight of the subject.

55. The method of claim 54, wherein the subject is a mammal selected from the group consisting of: a chicken, a rodent, a canine, a primate, an equine, a high value agricultural animal, and a human.

56. (canceled)
